(19) United States
(12) Patent Application Publication
(10) Pub. No.: US 2010/0081708 A1
(43) Pub. Date: Apr. 1, 2010

(54) ANTICOAGULANT COMPOUNDS

(75) Inventors: Maurice Petitou, Paris (FR); Guy Dubreucq, Lille (FR); Olivier Querolle, Le Plessis Hebert (FR); Sandrine Zameo, Paris (FR)

Correspondence Address: Pepper Hamilton LLP, 400 Berwyn Park, 899 Cassatt Road, Berwyn, PA, 19312-1183, US

(73) Assignee: ENDOTIS PHARMA, Loos (FR)

(21) Appl. No.: 12/444,198

(22) PCT Filed: Oct. 5, 2007
(86) PCT No.: PCT/IB2007/003938

§ 371 Date: Apr. 30, 2009

(30) Foreign Application Priority Data
Oct. 5, 2006 (EP) 06291556.6

(51) Int. Cl. A61K 31/7088; C07H 21/04; A61P 7/02

(52) U.S. Cl. 514/44 R; 536/23.1

(57) ABSTRACT

The present invention is concerned with anticoagulants (i.e. substances that stop blood from clotting). More specifically, the present invention is concerned with orally available antithrombic oligosaccharides.

All documents cited herein are incorporated by reference in their entirety.

### TECHNICAL FIELD

The present invention is concerned with anticoagulants (i.e. substances that stop blood from clotting). More specifically, the present invention is concerned with orally available antithrombic oligosaccharides.

### BACKGROUND ART

Heparin is an anticoagulant and is a natural sulphated polysaccharide that belongs to the family of glycosaminoglycans. Heparin acts as a controlling agent to prevent massive clotting of blood and, hence, runaway clot formation. The anticoagulant activity of heparin is reflected by its ability to accelerate the inhibition of several proteases in the blood-coagulation cascade including factor Xa and thrombin.

Heparin and heparin derived drugs inhibit the activity of factor Xa by attaching to a specific binding domain of antithrombin (AT). Once the heparin or heparin derived drugs are attached to the specific binding domain of antithrombin, they induce a conformational change in antithrombin (AT). It is the conformational change in AT that inhibits the activity of factor Xa. Investigations have shown that the lowest structural element that is capable of significantly binding AT, and inhibiting factor Xa, is a pentasaccharide.

Saccharides capable of binding to AT can be seen in European Patent 0 649 854, wherein a pentasaccharide chain- is said to be particularly advantageous at inhibiting factor Xa. Oligosaccharides capable of inhibiting thrombin, by binding to AT, are also disclosed in WO 98/03554 and WO 99/36443.

Additionally, U.S. Pat. No. 4,841,041 and U.S. Pat. No. 6,670,338 disclose pentasaccharides that have antithrombotic activity and anti-factor Xa activity. These pentasaccharides are said not to inactivate thrombin via inhibition of AT.

There are, however, problems associated with the use of heparins, which can be overcome by using low molecular weight heparins (LMWHs) that have improved pharmacokinetic properties (e.g. longer half-lives) relative to unfractionated heparins. Despite the pharmacokinetic advantages associated with the use of LMWHs, due to a lack of absorption when administered orally they can only be administered parenterally. Thus, in spite of their well established antithrombic properties, heparin and LMWHs, suffer from a reduced bioavailability following oral administration.

There therefore remains a need for the production of a heparin derivative that can be orally administered. Ideally, such anticoagulants should be stable under acidic conditions, such as those found in the stomach. It would also be particularly advantageous to produce compounds that can be obtained by a chemical synthesis, as opposed to natural products.

The present invention aims to produce oligosaccharide derivatives that act as anticoagulants and possess improved properties, such that they are capable of oral administration. It is a particular aim of the present invention to produce oligosaccharide derivatives that not only have an increased stability in the gastrointestinal tract, but are able to cross the intestinal membrane so that they can be absorbed in the intestine. It is particularly desirable to produce oligosaccharide derivatives that are capable of crossing the intestinal membrane because they overcome the oral bioavailability problems associated with heparins, heparin analogues and LMWHs. An additional aim of the present invention is to produce oligosaccharide derivatives that are particularly suitable to be adapted for use in galenic formulations, which arises from their enhanced lipophilicity.

### FIGURES

The figures show the absorption kinetic activity of exemplified compounds in plasma after Direct Intra Duodenal Injection, a process that is described in detail below. The compound numbers used in the figures corresponds to those examples described in the specification.

FIG. 1 shows the kinetic activity absorption of exemplified compounds of the invention. This figure also shows the kinetic activity absorption of a synthetic analogue of heparin, fondaparinux.

FIGS. 2 to 4 show data of the kinetic activity absorption of exemplified compounds of the invention.

FIG. 2 shows the kinetic activity absorption of the O-alkyl/family, wherein R₁₃, R₁₄ and R₁₅ are selected from the same functional group and the compound is derived from the 5S template.

FIG. 3 shows the kinetic activity absorption of O-alkyl/NHR family, wherein R₁₄ and R₁₅ are O-alkyl/O-arylalkyl and R₁₃ is NHR″ and the compound is derived from the 4S template.

FIG. 4 shows the kinetic activity absorption of O-alkyl/NHR family, wherein R₁₄ and R₁₅ are O-alkyl/O-arylalkyl and R₁₃ is NHR″ and the compound is derived from the 5S template.

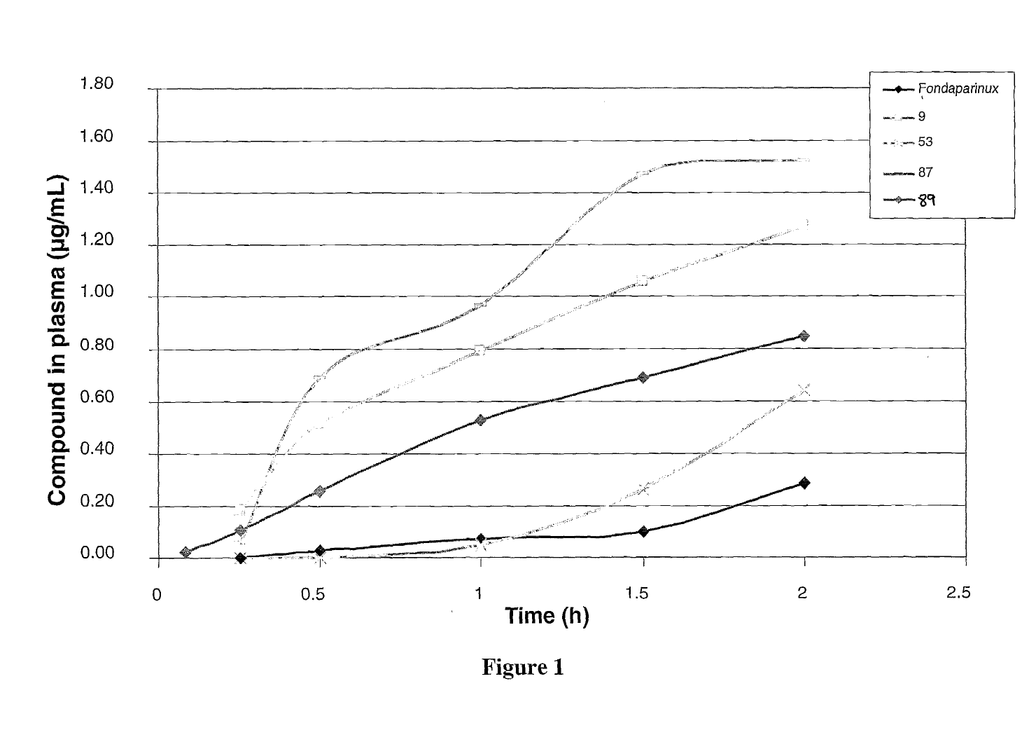

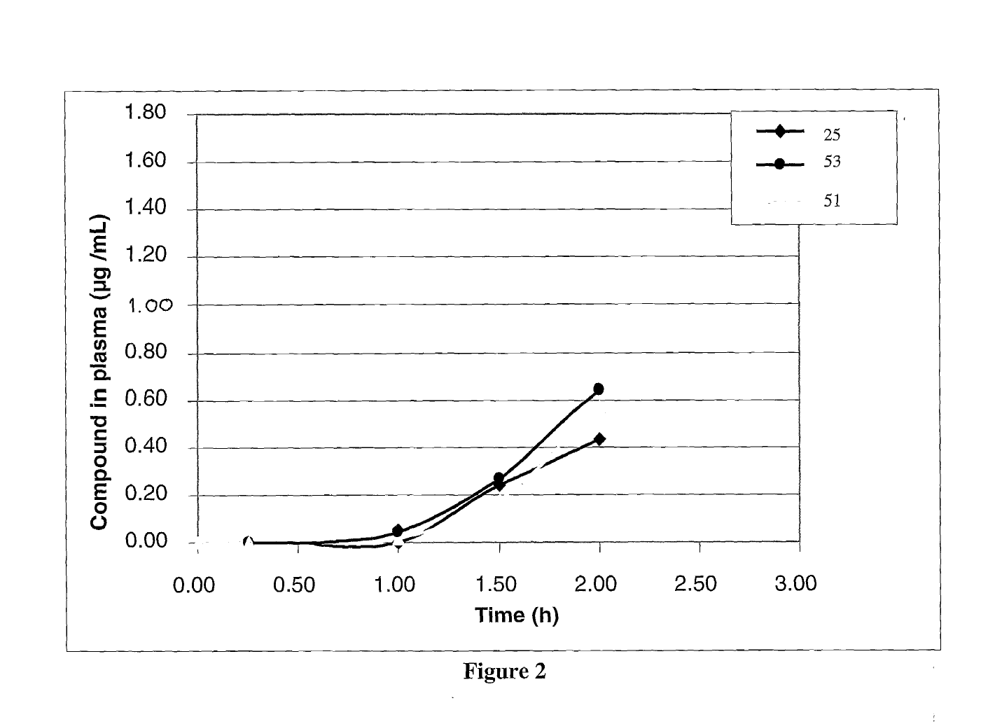

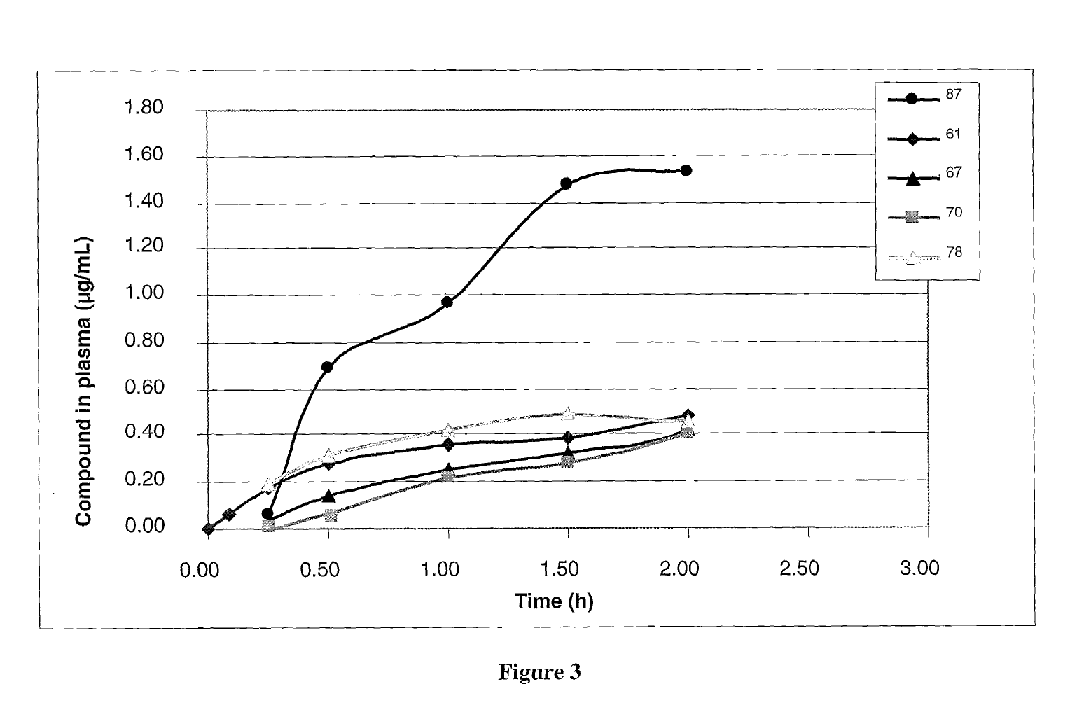

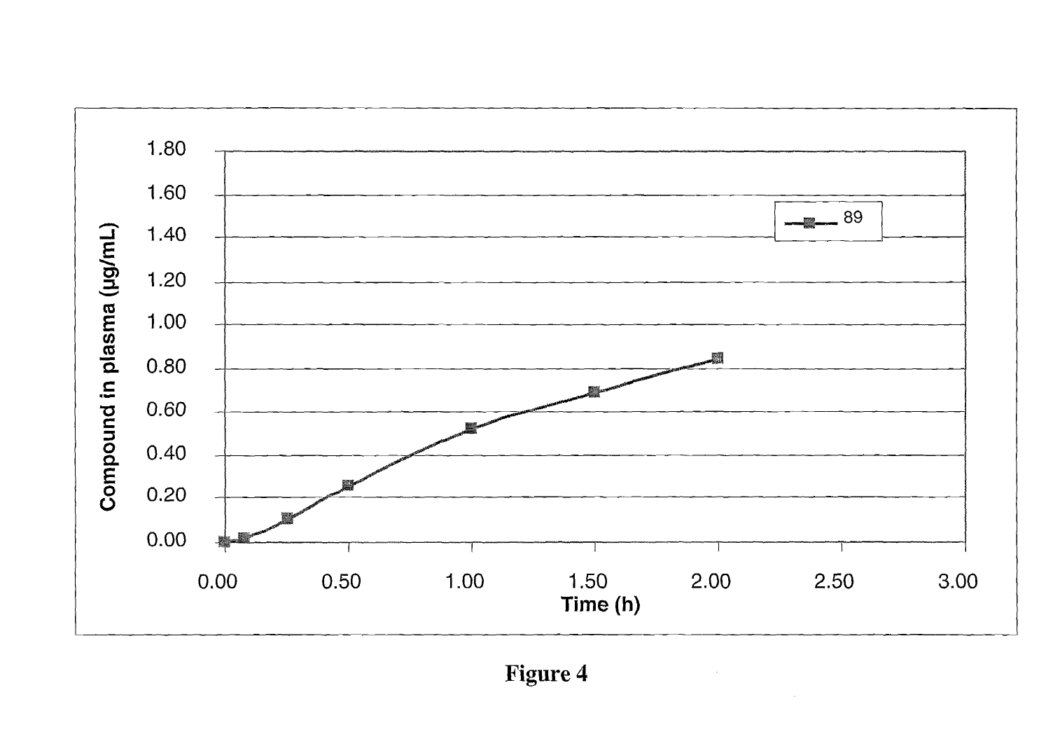

### DISCLOSURE OF THE INVENTION

According to one aspect of the present invention, there is provided a compound, a salt, solvate or prodrug thereof comprising a pentasaccharide that is capable of acting as an anticoagulant and inhibiting factor Xa.

### Pentasaccharide

Anticoagulants in the heparin family, such as LMWHs, are negatively charged and hydrophilic, which causes restrictions on their clinical use. Anticoagulants, such as LMWHs, typically have a low oral bioavailability which makes them unsuitable for oral administration.

The compounds of the present invention contain a reduced number of sulfate groups, while retaining a pharmacological effect (i.e. anticoagulant activity). The hydrophilicity problems that are encountered when using anticoagulants in the heparin family have been overcome by substituting hydroxyl groups with hydrophobic groups. These substitutions reduce the hydrophilicity of the molecule making it more suitable for oral administration.

The oligosaccharides of the present invention are of Formula (I):

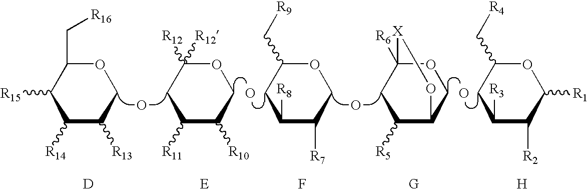

wherein:
   R₂, R₇, R₈ and R₁₆ are independently selected from the group consisting of: OSO₃H and NHSO₃H;
   R₆ and R₁₂ are each COOH;
   R₁, R₃, R₄, R₅, R₉, R₁₀, R₁₁, R₁₃, R₁₄ and R₁₅ are independently selected from the group consisting of: OH, OSO₃H, NH₂, NR′R″, N₃, O-alkyl, O-acyl, O-alkenyl, O-alkynyl, O-aryl, O-heteroaryl, O-heterocyclyl, O-aminoalkyl, O-alkylaryl, O-arylalkyl, O-alkylheteroaryl, O-alkylheterocyclyl;
   provided at least one of R₃, R₄, R₉, R₁₀, R₁₃, R₁₄ and R₁₅ is independently selected from the group consisting of: NH₂, NR′R″, N₃, O—(C₄₋₃₀-alkyl), O—(C₄₋₃₀-acyl), O-alkenyl, O-alkynyl, O-aryl, O-heteroaryl, O-heterocyclyl, O-aminoalkyl, O-alkylaryl, O-arylalkyl, O-alkylheteroaryl, O-alkylheterocyclyl;
   R_{12′} is selected from the group consisting of: H and alkyl;
   X is selected from the group consisting of: CH₂ and CH₂CH₂;
wherein R′ is independently selected from the group consisting of: H and alkyl;
wherein R″ is independently selected from the group consisting of: H, alkyl, alkenyl, alkoxy, C(O)alkyl, C(O)alkoxy, C(O)aryl, C(O)alkylaryl, C(O)arylalkyl and a lipophilic delivery moiety; and
wherein any of R′, R″, R₃, R₄, R₉, R₁₀, R₁₃, R₁₄ and R₁₅ are independently optionally substituted with one or more groups, preferably one, two or three of the groups, independently selected from alkyl, alkoxyalkyl, alkoxyaryl, alkynyl, heteroaryl, aryl, arylalkyl, alkaryl, COOH, COOalkyl, SH, S-alkyl, SO₂H, SO₂alkyl, SO₂aryl, SO₂alkaryl, P(OH)(O)₂, halo, haloalkyl, perhaloalkyl, OH, O-alkyl, ═O, NH₂, ═NH, NHalkyl, N(alkyl)₂, ═Nalkyl, NHC(O)alkyl, C(O)NH₂, C(O)NHalkyl, C(O)N(alkyl)₂, C(O)NHaryl, NO₂, ONO₂, CN, SO₂, SO₂NH₂, C(O)H, C(O)alkyl and wherein any of the aforementioned groups is optionally protected by a suitable protecting group;
or a salt, solvate or prodrug thereof.

In a preferred aspect of the invention, R₃, R₄, R₉, R₁₀, R₁₃, R₁₄ and R₁₅ are independently selected from the group consisting of: OH, OSO₃H, NH₂, NR′R″, N₃, O—(C₄₋₃₀-alkyl), O—(C₄₋₃₀-acyl), O-alkenyl, O-alkynyl, O-aryl, O-heteroaryl, O-heterocyclyl, O-aminoalkyl, O-alkylaryl, O-alkylheteroaryl, O-alkylheterocyclyl;
wherein any of R₃, R₄, R₉, R₁₀, R₁₃, R₁₄ and R₁₅ are independently optionally substituted with one or more groups independently selected from alkyl, alkoxyalkyl, alkoxyaryl, alkynyl, heteroaryl, aryl, arylalkyl, alkaryl, COOH, COOalkyl, SH, S-alkyl, SO₂H, SO₂alkyl, SO₂aryl, SO₂alkaryl, P(OH)(O)₂, halo, haloalkyl, perhaloalkyl, OH, O-alkyl, ═O, NH₂, ═NH, NHalkyl, N(alkyl)₂, ═Nalkyl, NHC(O)alkyl, C(O)NH₂, C(O)NHalkyl, C(O)N(alkyl)₂, NO₂, ONO₂, CN, SO₂, SO₂NH₂, C(O)H, C(O)alkyl and C(O)NHaryl and any of the aforementioned amine containing groups is optionally protected by a suitable protecting group, such as a benzyloxycarbonyl group.

In a preferred aspect of the invention, R₃, R₄, R₉, R₁₀, R₁₃, R₁₄ and R₁₅ are selected from the group consisting of: OH, N₃, NH₂, NR′R″, OSO₃H, O-alkyl, O-alkylaryl, O-aryl alkyl and O-acyl;
wherein any of R₃, R₄, R₉, R₁₀, R₁₃, R₁₄ and R₁₅ are independently optionally substituted with one or more groups independently selected from: OH, alkyl, halo, haloalkyl, perhaloalkyl, NH₂, NO₂, ONO₂ and any of the aforementioned amine containing groups is optionally protected by a benzyloxycarbonyl group.

In a preferred aspect of the invention, R₄, R₉, R₁₃, R₁₄ and R₁₅ are selected from the group consisting of: OH, N₃, OSO₃H, O-alkyl, O-alkylaryl, O-arylalkyl, NH₂, NR′R″ and O-acyl.

In a preferred aspect of the invention, the lipophilic delivery moiety is selected from the group consisting of: bile acids, sterols, non-steroidal anti-inflammatories, SNAD and SNAC.

In a preferred aspect of the invention, the R′ group is selected from any one of the groups consisting of: H and methyl.

In a preferred aspect of the invention, the R″ group is selected from the group consisting of: H, alkyl, alkenyl, alkoxy, C(O)alkyl, C(O)alkoxy, C(O)alkylaryl, C(O)arylalkyl, niflumic acid, mineral corticoids, preferably deoxycholoyl (DOCA), cholesterol, sodium N-[10-(2-hydroxybenzoyl)amino] decanoate (SNAD) and sodium N-[8-(2-hydroxybenzoyl)amino] caprylate (SNAC);
wherein the R″ group is optionally substituted with one or more groups, preferably one, two or three of the groups, independently selected from: alkyl, halo, haloalkyl, perhaloalkyl, NO₂, ONO₂ and wherein any of the aforementioned groups is optionally protected by a suitable protecting group, such as a nitrogen protecting group, for example, NH₂ can be protected by a benzyloxycarbonyl (Z) group (e.g. Z-amino).

In a further preferred aspect of the invention, the R″ group is selected from the group consisting of H, (benzyloxycarbonyl)aminohexanoyl (i.e. Z-aminohexanoyl), cyclopentylpropanoyl, DOCA, SNAD, SNAC, hexanoyl, hydrocinnamoyl, 3-cyclopentylpropanoyl, 3,5-bis(trifluoromethyl)benzoyl, (4-nitrooxy)butanoyl, dodecanoyl, arachidoyl, aminohexanoyl, niflumic acid.

In another preferred aspect of the invention, the R″ group is selected from the group consisting of: DOCA, C(O)alkyl, C(O)arylalkyl, H and C(O)alkyl;
wherein any of the aforementioned groups is optionally substituted with one or more NH₂ groups optionally protected by a benzyloxycarbonyl group.

In an alternative preferred aspect of the invention, R′ and R″ are both alkyl, preferably methyl.

Preferably, the oligosaccharides of the present invention are as follows:

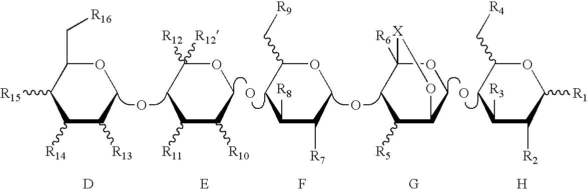

wherein:
   R₂, R₇, R₈ and R₁₆ are independently selected from the group consisting of: OSO₃H and NHSO₃H;
   R₆ and R₁₂ are each COOH;
   R₁, R₃, R₄, R₅, R₉, R₁₀, R₁₁, R₁₃, R₁₄ and R₁₅ are independently selected from the group consisting of: OH, OSO₃H, NH₂, O-alkyl, O-acyl, O-alkenyl, O-alkynyl, O-aryl, O-heteroaryl, O-heterocyclyl, O-aminoalkyl, O-alkylaryl, O-alkylheteroaryl, O-alkylheterocyclyl;
   provided at least one of R₃, R₄, R₉, R₁₀, R₁₃, R₁₄ and R₁₅ is independently selected from the group consisting of: NH₂, O—(C₄₋₃₀-alkyl), O—(C₄₋₃₀-acyl), O-alkenyl, O-alkynyl, O-aryl, O-heteroaryl, O-heterocyclyl, O-aminoalkyl, O-alkylaryl, O-alkylheteroaryl, O-alkylheterocyclyl;
   R_{12′} is selected from the group consisting of: H and alkyl;
   X is selected from the group consisting of: CH₂ and CH₂CH₂; and
wherein any of R₃, R₄, R₉, R₁₀, R₁₃, R₁₄ and R₁₅ are independently optionally substituted with one or more groups, preferably one, two or three of the groups, independently selected from alkyl, alkoxyalkyl, alkoxyaryl, alkynyl, heteroaryl, aryl, arylalkyl, alkaryl, COOH, COOalkyl, SH, S-alkyl, SO₂H, SO₂alkyl, SO₂aryl, SO₂alkaryl, P(OH)(O)₂, halo, haloalkyl, perhaloalkyl, OH, O-alkyl, ═O, NH₂, ═NH, NHalkyl, N(alkyl)₂, ═Nalkyl, NHC(O)alkyl, C(O)NH₂, C(O)NHalkyl, C(O)N(alkyl)₂, C(O)NHaryl, NO₂, CN, SO₂, SO₂NH₂, C(O)H, C(O)alkyl;
or a salt, solvate or prodrug thereof.

More preferably, the oligosaccharide of the present invention is of Formula (II):

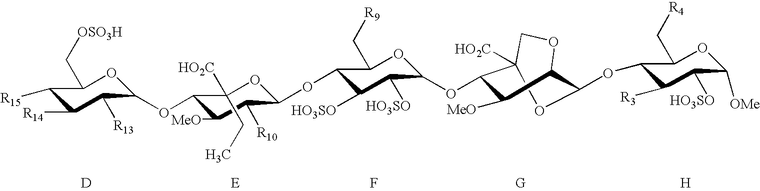

In a preferred aspect of the present invention, the group R₃ is OSO₃H.

In another preferred aspect of the present invention, the groups R₁, R₅ and R₁₁ are each O-alkyl.

In another preferred aspect of the present invention, the groups R₁, R₅, R₁₀ and R₁₁ are each O-alkyl. Preferably, these O-alkyl group is OMe.

In another preferred aspect of the present invention, the groups R₂, R₇ and R₈ are each OSO₃H.

In another preferred aspect of the present invention, the group R₃ is selected from the groups OSO₃H and O-alkyl. Preferably, the O-alkyl group is OMe.

In another preferred aspect of the present invention, the group R_{12′} is CH₂CH₃.

In another preferred aspect of the present invention, X is CH₂.

In another preferred aspect of the present invention, the groups R₁₄ and R₁₅ are selected from the group consisting of: OH, O-alkyl and O-arylalkyl.

Preferably, R₁₄ and R₁₅ are selected from: OH, O-methyl, O-butyl, O-hexyl and O-benzyl.

In another preferred aspect of the present invention, the group R₁₃ is selected from the group consisting of: O-alkyl, O-arylalkyl, N₃, NH₂ and NR′R″,
wherein R′ is selected from H and R″ is selected from the group consisting of C(O)alkyl and C(O)alkylaryl and any of the aforementioned groups is optionally substituted with one or more NH₂ groups, which can be optionally protected by a suitable protecting group, such as benzyloxycarbonyl.

Preferably, R₁₃ is selected from: O-methyl, O-hexyl, O-benzyl, N₃, NH₂, NH(Z-aminohexanoyl), NH(3-cyclopentylpropanoyl) and NHhydrocinnamoyl.

In another preferred aspect of the present invention, the group R₉ is selected from the group consisting of: OH, OSO₃H, N₃, O-alkyl and NR′R″, wherein R′ is hydrogen and R″ is selected from DOCA. Preferably, R₉ is selected from: OH, OSO₃H, N₃, O-hexyl and NDOCA.

In another preferred aspect of the present invention, the group R₄ is selected from the group consisting of: OH, OSO₃H, N₃, O-alkyl and NR′R″, wherein R′ is hydrogen and R″ is C(O)alkylaryl.

Preferably, R₄ is selected from: OH, OSO₃H, N₃, O-hexyl and NHhydrocinnamoyl.

In another preferred aspect of the present invention, the group R₁₀ is OCH₃.

In another preferred aspect of the present invention, the group R₁₃ is NH₂.

In another preferred aspect of the present invention, the groups R₄, R₉, R₁₄ and R₁₅ are each OH.

In another aspect of the present invention, the groups R₃, R₄, R₉, R₁₀, R₁₃, R₁₄ and R₁₅ are independently selected from the group consisting of: OH, OSO₃H, NH₂, O—(C₄₋₃₀-alkyl), O—(C₄₋₃₀-acyl), O-alkenyl, O-alkynyl, O-aryl, O-heteroaryl, O-heterocyclyl, O-aminoalkyl, O-alkylaryl, O-alkylheteroaryl, O-alkylheterocyclyl;
wherein any of R₃, R₄, R₉, R₁₀, R₁₃, R₁₄ and R₁₅ are independently optionally substituted with one or more groups, preferably one, two or three of the groups, independently selected from alkyl, alkoxyalkyl, alkoxyaryl, alkynyl, heteroaryl, aryl, arylalkyl, alkaryl, COOH, COOalkyl, SH, S-alkyl, SO₂H, SO₂alkyl, SO₂aryl, SO₂alkaryl, P(OH)(O)₂, halo, haloalkyl, perhaloalkyl, OH, O-alkyl, ═O, NH₂, ═NH, NHalkyl, N(alkyl)₂, ═Nalkyl, NHC(O)alkyl, C(O)NH₂, C(O)NHalkyl, C(O)N(alkyl)₂, NO₂, CN, SO₂, SO₂NH₂, C(O)H, C(O)alkyl and C(O)NHaryl.

Preferably, the groups R₃, R₄, R₉, R₁₀, R₁₃, R₁₄ and R₁₅ are independently selected from: OH, OSO₃H, NH₂, O—(C₄₋₃₀-alkyl), O—(C₄₋₃₀-acyl), O-heterocyclyl, O-aryl, O-alkylaryl;
wherein any of R₃, R₄, R₉, R₁₀, R₁₃, R₁₄ and R₁₅ are independently optionally substituted with one or more groups, preferably one, two or three of the groups, independently selected from halo, haloalkyl, perhaloalkyl, OH, O-alkyl, ═O, alkyl, alkoxyalkyl, alkoxyaryl, alkynyl, arylalkyl, alkaryl, heteroaryl and aryl.

More preferably, the groups R₃, R₄, R₉, R₁₀, R₁₃, R₁₄ and R₁₅ are independently selected from: O-butyl, nonanoyl, (4-tert-butyl)benzyloxy, 3-cyclopentylpropanoyl, hexanoyl, 2,2-dimethylpropyloxy, 4-chlorobenzyloxy, OH and deoxycholoyl.

In a preferred aspect of the present invention, the groups R₃, R₄, R₉, R₁₀, R₁₃, R₁₄ and R₁₅ are each O-butyl.

In another preferred aspect of the present invention, the groups R₃, R₄, R₉, R₁₀, R₁₃, R₁₄ and R₁₅ are each nonanoyl.

In a preferred aspect of the present invention, the groups R₃, R₁₀, R₁₃, R₁₄ and R₁₅ are each (4-tert-butyl)benzyloxy.

In another preferred aspect of the present invention, the groups R₃, R₁₀, R₁₃, R₁₄ and R₁₅ are each hexanoyl.

In another preferred aspect of the present invention, the groups R₃, R₁₀, R₁₃, R₁₄ and R₁₅ are each 4-chlorobenzyloxy).

In another preferred aspect of the present invention, the groups R₃, R₁₀, R₁₃, R₁₄ and R₁₅ are each OH.

In a preferred aspect of the present invention, the groups R₄ and R₉ are each 3-cyclopentylpropanoyl.

In another preferred aspect of the present invention, the groups R₄ and R₉ are each 2,2-dimethylpropyloxy.

In a preferred aspect of the present invention, the groups R₄ and R₉ are each OH.

In a preferred aspect of the present invention, the groups R₄ and R₉ are each deoxycholoyl.

In the present specification, the groups —COOH, —OSO₃H and —NHSO₃H are represented in their acid form. It will be understood the representation in their acid form also extends to their salt form. In a preferred embodiment these groups are in their salt form, more preferably in their sodium salt form.

It will be appreciated that the pentasaccharide can exist in a variety of stereochemical forms, which will be apparent to one skilled in the art. Positions of variable stereochemistry include those indicated with wavy lines. Except where specifically indicated, the present invention extends to all such stereochemical forms.

Advantageously, the G monosaccharide unit of the oligosaccharide has the following conformation:

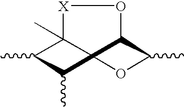

Preferably, the D, E, F and H monosaccharide units of the oligosaccharide have the D-gluco stereochemistry:

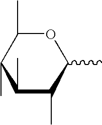

Additionally, it is preferred that the G monosaccharide unit of the oligosaccharide has the following stereochemistry:

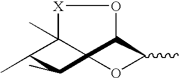

In a preferred aspect of the invention, the groups R₁, R₅ and R₁₁ are each OMe.

In another preferred aspect of the invention, the groups R₂, R₇, R₈ and R₁₆ are each OSO₃H.

In another preferred aspect of the invention, the group X is CH₂.

In another preferred aspect of the invention, the group R_{12′} is CH₂CH₃.

For the avoidance of doubt, the present invention extends to any combination of the aforementioned aspects.

In a further aspect of the present invention, a pharmaceutical composition is provided comprising a pentasaccharide, as described in the present invention, and a pharmaceutically acceptable diluent or carrier.

The present invention also provides a method of making a pharmaceutical composition, comprising mixing the pentasaccharide of the present invention with a pharmaceutically acceptable diluent or carrier.

In a further aspect of the present invention, there is provided use of a pentasaccharide, as described in the present invention, in therapy.

In another aspect of the invention, there is provided the use of a pentasaccharide, as defined in the present invention, in the manufacture of a medicament for the treatment of a blood clotting disorder.

The present invention also provides a method of treating a blood clotting disorder in a human or animal subject comprising administering to the human or animal subject a therapeutically effective amount of a pentasaccharide, as defined in the present invention.

In another aspect of the invention, the medicament as described above, can be used for oral administration. Preferably, the method of treatment also involves oral administration.

Preferably, the blood clotting disorder is selected from: deep vein thromboembolism including deep vein thrombosis and pulmonary embolism, post surgical prophylaxis of deep venous thrombosis, coronary syndromes, myocardial infarction and stroke.

### DEFINITIONS

### Pharmaceutical Compositions

The compounds of the present invention may also be present in the form of pharmaceutically acceptable salts. For use in medicine, the salts of the compounds of this invention refer to non-toxic “pharmaceutically acceptable salts.” FDA approved pharmaceutically acceptable salt forms (Gould, P. L. International J. Pharm., 1986, 33, 201-217; Berge, S. M. et al. J. Pharm. Sci., 1977, 66(1), 1-19) include pharmaceutically acceptable acidic/anionic or basic/cationic salts.

Pharmaceutically acceptable salts of the acidic or basic compounds of the invention can of course be made by conventional procedures, such as by reacting the free base or acid with at least a stoichiometric amount of the desired salt-forming acid or base.

Pharmaceutically acceptable salts of the acidic compounds of the invention include salts with inorganic cations such as sodium, potassium, calcium, magnesium, zinc, and ammonium, and salts with organic bases. Suitable organic bases include N-methyl-D-glucamine, arginine, benzathine, diolamine, olamine, procaine and tromethamine.

Pharmaceutically acceptable salts of the basic compounds of the invention include salts derived from organic or inorganic acids. Suitable anions include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulfate, napsylate, nitrate, oleate, pamoate, phosphate, polygalacturonate, stearate, succinate, sulfate, sulfosalicylate, tannate, tartrate, terephthalate, tosylate and triethiodide. Hydrochloride salts are particularly preferred.

The invention also comprehends derivative compounds (“pro-drugs”) which are degraded in vivo to yield the species of Formula (I). Pro-drugs are usually (but not always) of lower potency at the target receptor than the species to which they are degraded. Pro-drugs are particularly useful when the desired species has chemical or physical properties, which make its administration difficult or inefficient. For example, the desired species may be only poorly soluble, it may be poorly transported across the mucosal epithelium, or it may have an undesirably short plasma half-life. Further discussion of pro-drugs may be found in Stella, V. J. et al. “Prodrugs”, Drug Delivery Systems, 1985, 112-176, Drugs, 1985, 29, 455-473 and “Design of Prodrugs”, ed. H. Bundgaard, Elsevier, 1985.

The compounds described in the claims having an amino group may be derivatised with a ketone or an aldehyde such as formaldehyde to form a Mannich base. This will hydrolyse with first order kinetics in aqueous solution. In addition, the compounds described in the claims having one or more free hydroxy groups may be esterified in the form of a pharmaceutically acceptable ester. This may be convertible, by solvolysis, under physiological conditions to the compounds of the present invention having free hydroxy groups.

Thus, in the methods of treatment of the present invention, the term “administering” shall encompass the treatment of the various disorders described with the compound specifically disclosed or with a compound which may not be specifically disclosed, but which converts to the specified compound in vivo after administration to the subject.

It is anticipated that the compounds of the invention can be administered by oral or parenteral routes, including intravenous, intramuscular, intraperitoneal, subcutaneous, transdermal, rectal and topical administration, and inhalation. Oral administration of the compounds of the present invention is particularly preferred.

For oral administration, the compounds of the invention will generally be provided in the form of tablets or capsules or as an aqueous solution or suspension.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate and lactose. Corn starch and alginic acid are suitable disintegrating agents. Binding agents may include starch and gelatine. The lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatine capsules in which the active ingredient is mixed with a solid diluent and soft gelatine capsules wherein the active ingredient is mixed with water or an oil such as peanut oil, liquid paraffin or olive oil.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions of the present invention may, in particular, comprise more than one compound (multiple) of the present invention, e.g., two or more compounds. The invention also provides a pharmaceutical preparation or system, comprising (a) a first compound, which is a compound of the invention; and (b) a second pharmaceutical compound. Said multiple compounds of the invention or said first and second compounds are formulated either in admixture or as separate compositions, e.g. for simultaneous though separate, or for sequential administration (see below).

### Modes of Administration

The compounds of the present invention can be delivered directly or in pharmaceutical compositions containing excipients (see above), as is well known in the art. The present methods of treatment involve administration of a therapeutically effective amount of a compound of the present invention to a subject.

The term “therapeutically effective amount” or “therapeutically effective dose” as used herein refers to an amount of a compound according to the present invention needed to: treat; ameliorate; prevent the targeted disease condition; exhibit a detectable therapeutic or preventative effect; prolong survival of a patient. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The therapeutically effective amount or therapeutically effective dose is the amount of the compound or pharmaceutical composition that will elicit the biological or medical response of a tissue, system, animal, or human that is being sought by the researcher, veterinarian, medical doctor, or other clinician. For example, anticoagulant activity and treatment of blood clotting disorders, e.g., deep vein thromboembolism including deep vein thrombosis and pulmonary embolism, post surgical deep venous thrombosis, coronary syndromes, myocardial infarction, stroke, etc.

Dosages preferably fall within a range of circulating concentrations that includes the ED50 with little or no toxicity. Dosages may vary within this range depending upon the dosage form employed and/or the route of administration utilised. The exact formulation, route of administration, dosage, and dosage interval should be chosen according to methods known in the art, in view of the specifics of a patient's condition.

Dosage amount and interval may be adjusted individually to provide plasma levels of the active moiety that are sufficient to achieve the desired effects, i.e., minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

In general, the therapeutically effective dose/amount can be estimated by using 30 conventional methods and techniques that are known in the art. Initial doses used in animal studies (e.g. non-human primates, mice, rabbits, dogs, or pigs) may be based on effective concentrations established in cell culture assays. The animal model may also be used to determine the appropriate concentration range and route of administration. Such information can then be used to determine useful doses and routes for administration in human patients.

The specific dosage level required for any particular patient will depend on a number of factors, including severity of the condition being treated, the route of administration, the general health of the patient (i.e. age, weight and diet), the gender of the patient, the time and frequency of administration, judgement of the prescribing physician and tolerance/response to therapy. In general, however, the daily dose (whether administered as a single dose or as divided doses) will be in the range 0.01 to 500 mg per day, more usually from 0.1 to 50 mg per day, and most usually from 1 to 10 mg per day. Alternatively, dosages can be administered per unit body weight and, in this instance, a typical dose will be between 0.001 mg/kg and 3 mg/kg, especially between 0.01 mg/kg and 0.2 mg/kg, between 0.02 mg/kg and 0.1 mg/kg.

An effective and convenient route of administration and an appropriate formulation of the compounds of the invention in pharmaceutical compositions (see above) may also be readily determined by routine experimentation. Various formulations and drug delivery systems are available in the art (see, e.g., Gennaro A R (ed.). Remington: The Science and Practice of Pharmacy. Lippincott Williams & Wilkins. 21st edition. Jul. 3, 2005 and Hardman J G, Limbird L E, Alfred G. Gilman A G. Goodman & Gilman's The Pharmacological Basis of Therapeutics. McGraw-Hill; 10th edition. Aug. 13, 2001).

As mentioned above, suitable routes of administration may, for example, include vaginal, rectal, intestinal, oral, nasal (intranasal), pulmonary or other mucosal, topical, transdermal, ocular, aural, and parenteral administration.

An advantage of the compounds of the present invention is that they are particularly suitable for oral administration.

Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising an agent of the invention formulated in a compatible pharmaceutical carrier may also be prepared, placed in an appropriate container, and labelled for treatment of an indicated condition.

In addition to the above, the compounds of the present invention are particularly suitable for use in galenic formulations due to their lipophilicity. In their most basic form, galenic formulations typically involve mixing two compounds, one of which is poorly orally available, to form a formulation. The resultant mixture of compounds has an enhanced oral availability because the compounds are able to cross the intestinal membrane more efficiently due to their increased lipophilicity. However, galenic formulations are well known to the skilled person and the differences between such formulations and oral delivery per se is described in, for example, Motlekar, N. A. and al. Journal of Controlled Release 2006, 113, 91-101. Additionally, the skilled person would also be aware that galenic formulations could be used in conjunction with heparins and LMWHs, see Goldberg, M. and al., Nature reviews 2003, 2, 289-295, Bernkop-Schnürch, A. and al. Expert Opin. Drug Deliv. 2004, 1, 87-98, Bernkop-Schnürch, A. and al. Journal of Pharmaceutical Science 2005, 94 (5), 966-972 and Arbit, E. and al. Thrombosis Journal 2006, 4 (6), 1-25 (Emisphere technology) for example. Although these documents discuss the use of galenic formulations in conjunction with heparin and LMWHs, it has not been previously appreciated that they would be particularly advantageous if glaenic compositions are used with compounds similar to those of the present invention i.e. synthetic lipophilic oligosaccharides.

### Chemical Definitions

Formulaic representation of apparent orientation of a functional group is not necessarily intended to represent actual orientation. Thus, for example, a divalent amide group represented as C(O)NH is also intended to cover NHC(O).

In the interests of simplicity, terms which are normally used to refer to monovalent groups (such as “alkyl” or “alkenyl”) are also used herein to refer to divalent, trivalent or tetravalent bridging groups which are formed from the corresponding monovalent group by the loss of one or more hydrogen atom(s). Whether such a term refers to a monovalent group or to a polyvalent group will be clear from the context. Where a polyvalent bridging group is formed from a cyclic moiety, the linking bonds may be on any suitable ring atom, according to the normal rules of valency.

Where any particular moiety is substituted, for example an imidazole group comprising a substituent on the heteroaryl ring, unless specified otherwise, the term “substituted” contemplates all possible isomeric forms. For example, substituted imidazole includes all of the following permutations:

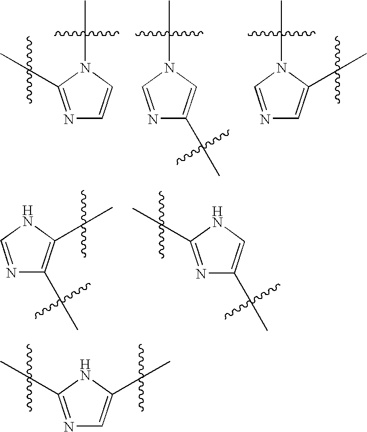

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more and the same or different for appropriate organic compounds. For purposes of this invention, heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

The terms “comprising” and “comprises” means “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

“Optional” or “optionally” means that the subsequently described event of circumstances may or may not occur, and that the description includes instances where said event or circumstance occurs and instances in which it does not.

“May” means that the subsequently described event of circumstances may or may not occur, and that the description includes instances where said event or circumstance occurs and instances in which it does not.

Where the compounds according to this invention have at least one chiral centre, they may accordingly exist as enantiomers. Where the compounds possess two or more chiral centres, they may additionally exist as diastereomers. Where the processes for the preparation of the compounds according to the invention give rise to mixture of stereoisomers, these isomers may be separated by conventional techniques such as preparative chromatography. The compounds may be prepared in racemic form or individual enantiomers may be prepared by standard techniques known to those skilled in the art, for example, by enantiospecific synthesis or resolution, formation of diastereomeric pairs by salt formation with an optically active acid, followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column. It is to be understood that all such isomers and mixtures thereof are encompassed within the scope of the present invention.

Where a group comprises two or more moieties defined by a single carbon atom number, for example, C₂₋₂₀-alkoxyalkyl, the carbon atom number indicates the total number of carbon atoms in the group.

As used herein, the term “lipophilic delivery moiety” is used to refer to a radical that corresponds to a lipophilic delivery agent. Preferably, a lipophilic delivery agent is selected from the group consisting of: bile acids, sterols, non-steroidal anti-inflammatory or compounds such as sodium N-[10-(2-hydroxybenzoyl)amino] decanoate (SNAD) and sodium N-[8-(2-hydroxybenzoyl)amino] caprylate (SNAC).

As used herein, the term “lipophilic” refers to a moiety that has a partition co-efficient octanol/water that is greater than or equal to that of n-butane.

As used herein, the term “bile acid” includes moieties that are produced in the liver by the oxidation of cholesterol, conjugated (with either the amino acid taurine or glycine, or a sulfate, or a glucuronide) and are stored in the gallbladder. Typical examples of bile acids include cholic acid, taurocholic acid, glycocholic acid, deoxycholic acid, and chenodeoxycholic acid. In the present invention, deoxycholic acid is a particularly preferred bile acid.

As used herein, the term “sterol” preferably refers to compounds that fall within a subgroup of steroids and are amphipathic lipids synthesised from acetyl-coenzyme A. The sterols used in the present invention can be sterols of plants (i.e. phytosterols, such as campesterol, sitosterol, and stigmasterol), or they can be sterols of animals (i.e. zoosterols, such as cholesterol and some steroid hormones). A preferable sterol used in the present invention is cholesterol.

As used herein, the term “non-steroidal anti-inflammatory” preferably refers to compounds that are non-competitive inhibitors of calcium-activated chloride currents. For example, a suitable non-steroidal anti-inflammatory is niflumic acid.

As used herein, the term “protecting group” refers to functional groups that are well known to the skilled person and are described in “Protecting Groups in Organic Synthesis” 3rd Edition T. W. Greene and P. G. Wuts, Wiley-Interscience, 1999. For example, benzyloxycarbonyl, which can be removed by acidolysis with strong acids or by catalytic hydrogenation producing carbon dioxide and toluene as side products, is a common amine protecting group. An alternative amine protecting group is tert-butoxy carbonyl (BOC), which can be removed by treatment with an acid, such as trifluoroacetic acid or hydrogen chloride in an organic solvent such as dichloromethane.

The skilled person will appreciate that, in addition to protecting nitrogen atoms of amines, as discussed above, it may be necessary to protect, and deprotect, other functional groups with suitable protecting groups, such as, for example, hydroxy groups. Methods for deprotection of any particular protecting group will depend on the protecting group that is used and the functional group that is being protected. For examples of protection/deprotection methodology see “Protective groups in Organic synthesis”, T. W. Greene and P. G. M. Wutz.

As used herein, the term “heteroatom” includes N, O, S, P, Si and halogen (including F, Cl, Br and I). In the context of a hydrocarbon chain interrupted by one or more heteroatoms, the term “heteroatoms” includes N, O and S.

The term “halogen” or “halo” is used herein to refer to any of fluorine, chlorine, bromine and iodine. Most usually, however, halogen substituents in the compounds of the invention are chlorine, bromine and fluorine substituents. Groups such as halo(alkyl) include mono-, di-, tri- and per-halo substituted alkyl groups. Moreover, the halo substitution may be at any position in the alkyl chain. “Perhalo” means completely halogenated, e.g., trihalomethyl and pentachloroethyl.

As used herein, the term “alkyl” refers to a cyclic, straight or branched saturated monovalent hydrocarbon radical, having the number of carbon atoms as indicated. For example, the term “C₁₋₃₀-alkyl” includes C₁, C₂, C₃, C₄, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀ alkyl groups. By way of non-limiting example, suitable alkyl groups include methyl, ethyl, propyl, iso-propyl, butyl, iso-butyl, tert-butyl, pentyl, hexyl, octyl, nonyl, dodecyl, eicosyl, cyclopropyl, cyclobutyl, cyclopentyl, cyclohexyl, methylcyclohexyl, dimethylcyclohexyl, trimethylcyclohexyl, cycloheptyl, cyclooctyl, cyclononyl, cyclododecyl, spiroundecyl, bicyclooctyl and adamantyl, cyclopropylmethyl, cyclopropylethyl, cyclobutylmethyl, cyclobutylethyl, cyclopentylmethyl, cyclopentylethyl, cyclopentylpropyl, cyclohexylmethyl, cyclohexylethyl, cyclohexylpropyl, cyclohexylbutyl, methylcyclohexylmethyl, dimethylcyclohexylmethyl, trimethylcyclohexylmethyl, cycloheptylmethyl, cycloheptylethyl, cycloheptylpropyl, cycloheptylbutyl and adamantylmethyl. Preferred ranges of alkyl groups of the present invention are: C₁₋₃₀-alkyl, C₂₋₂₈-alkyl, C₃₋₂₆-alkyl, C₄₋₂₄ alkyl, C₄₋₂₂-alkyl, C₅₋₂₀-alkyl, C₅₋₁₈-alkyl, C₆₋₁₆-alkyl, C₇₋₁₄-alkyl and C₈₋₁₂-alkyl. Preferred ranges in cycloalkyl groups are: C₄₋₃₀, C₄₋₂₀, C₄₋₁₅ and C_{5-13.}

As used herein, the term “alkenyl” refers to a cyclic, straight or branched unsaturated monovalent hydrocarbon radical, having the number of carbon atoms as indicated, and the distinguishing feature of a carbon-carbon double bond. For example, the term “C₂₋₃₀-alkenyl” includes C₂, C₃, C₄, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀ alkenyl groups. By way of non-limiting example, suitable alkenyl groups include ethenyl, propenyl, butenyl, penentyl, hexenyl, octenyl, nonenyl, dodecenyl and eicosenyl, wherein the double bond may be located anywhere in the carbon chain. Preferred ranges of alkenyl groups of the present invention are: C₂₋₃₀-alkenyl, C₂₋₂₈ alkenyl, C₃₋₂₆ alkenyl, C₄₋₂₄-alkenyl, C₅₋₂₂-alkenyl, C₅₋₂₀-alkenyl, C₆₋₁₈-alkenyl, C₆₋₁₆-alkenyl, C₇₋₁₄-alkenyl and C₈₋₁₂-alkenyl. Preferred ranges in cycloalkenyl groups are: C₄₋₃₀, C₄₋₂₀, C₅₋₁₅ and C₆₋₁₃.

As used herein, the term “alkynyl” refers to a cyclic, straight or branched unsaturated monovalent hydrocarbon radical, having the number of carbon atoms as indicated, and the distinguishing feature of a carbon-carbon triple bond. For example, the term “C₂₋₃₀ alkynyl” includes C₂, C₃, C₄, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀ alkynyl groups. By way of non-limiting example, suitable alkynyl groups include ethynyl, propynyl, butynyl, penynyl, hexynyl, octynyl, nonynyl, dodycenyl and eicosynyl, wherein the triple bond may be located anywhere in the carbon chain. Preferred ranges of alkynyl groups of the present invention are: C₂₋₃₀-alkynyl, C₂₋₂₈-alkynyl, C₃₋₂₆-alkynyl, C₄₋₂₄-alkynyl, C₄₋₂₂-alkynyl, C₅₋₂₀-alkynyl, C₅₋₁₈-alkynyl, C₆₋₁₆-alkynyl, C₇₋₁₄-alkynyl and C₈₋₁₂-alkynyl. Preferred ranges in cycloalkenyl groups are: C₈₋₃₀, C₉₋₂₀, C₅₋₁₅ and C₁₀₋₁₃.

Alkoxy refers to the group “alkyl-O—”, where alkyl is as defined above. By way of non-limiting example, suitable alkoxy groups include methoxy, ethoxy, propoxy, butoxy, pentoxy and hexoxy.

As used herein, the term “alkoxyalkyl” refers to an alkyl group having an alkoxy substituent. Binding is through the alkyl group. The alkyl moiety may be cyclic, straight or branched. The alk and alkyl moieties of such a group may be substituted as defined above, with regard to the definition of alkyl. By way of non-limiting example, suitable alkoxyalkyl groups include methoxymethyl, methoxyethyl, ethoxymethyl, ethoxyethyl, methoxypropyl and ethoxypropyl.

As used herein, the term “alkoxyaryl” refers to an aryl group having an alkoxy substituent. Binding is through the aryl group. The alkoxy and aryl moieties of such a group may be substituted as defined herein, with regard to the definitions of alkoxy and aryl. The alkyl moiety may be cyclic, straight or branched. By way of non-limiting example, suitable alkoxyaryl groups include methoxyphenyl, ethoxyphenyl, dimethoxyphenyl and trimethoxyphenyl.

As used herein, the term “aryl” refers to monovalent aromatic carbocyclic radical having one, two, three, four, five or six rings, preferably one, two or three rings, which may be fused or bicyclic. Preferably, the term “aryl” refers to an aromatic monocyclic ring containing 6 carbon atoms, which may be substituted on the ring with 1, 2, 3, 4 or 5 substituents as defined herein; an aromatic bicyclic or fused ring system containing 7, 8, 9 or 10 carbon atoms, which may be substituted on the ring with 1, 2, 3, 4, 5, 6, 7, 8 or 9 substituents as defined herein; or an aromatic tricyclic ring system containing 10, 11, 12, 13 or 14 carbon atoms, which may be substituted on the ring with 1, 2, 3, 4, 5, 6, 7, 8, 9, 10, 11, 12 or 13 substituents as defined herein. By way of non-limiting example, suitable aryl groups include phenyl, biphenyl, binaphthyl, indanyl, phenanthryl, fluoryl, flourenyl, stilbyl, benzylphenanthryl, acenaphthyl, azulenyl, phenylnaphthyl, benzylfluoryl, tetrahydronaphthyl, perylenyl, picenyl, chrysyl, pyrenyl, tolyl, chlorophenyl, dichlorophenyl, trichlorophenyl, methoxyphenyl, dimethoxyphenyl, trimethoxyphenyl, fluorophenyl, difluorophenyl, trifluorophenyl, nitrophenyl, dinitrophenyl, trinitrophenyl, aminophenyl, diaminophenyl, triaminophenyl, cyanophenyl, chloromethylphenyl, tolylphenyl, xylylphenyl, chloroethylphenyl, trichloromethylphenyl, dihydroindenyl, benzocycloheptyl and trifluoromethylphenyl. Preferred ranges of aryl groups of the present invention are: C₆₋₂₅-aryl, C₆₋₂₃-aryl, C₆₋₂₀-aryl, C₆₋₁₈-aryl, C₆₋₁₅-aryl, C₆₋₁₂-aryl, C₆₋₁₀-aryl, C₆₋₉-aryl, C₆₋₈-aryl and C₆₋₇-aryl.

The term “heteroaryl” refers to a monovalent unsaturated aromatic heterocyclic radical having one, two, three, four, five or six rings, preferably one, two or three rings, which may be fused or bicyclic. Preferably, “heteroaryl” refers to an aromatic monocyclic ring system containing five members of which at least one member is a N, O or S atom and which optionally contains one, two or three additional N atoms, an aromatic monocyclic ring having six members of which one, two or three members are a N atom, an aromatic bicyclic or fused ring having nine members of which at least one member is a N, O or S atom and which optionally contains one, two or three additional N atoms or an aromatic bicyclic ring having ten members of which one, two or three members are a N atom. By way of non-limiting example, suitable heteroaryl groups include furanyl, pryingly, pyridyl, phthalimido, thiophenyl, pyrrolyl, imidazolyl, pyrazolyl, thiazolyl, isothiazolyl, oxazolyl, oxadiazolyl, pyronyl, pyrazinyl, tetrazolyl, thionaphthyl, benzofuranyl, isobenzofuryl, indolyl, oxyindolyl, isoindolyl, indazolyl, indolinyl, azaindolyl, isoindazolyl, benzopyranyl, coumarinyl, isocoumarinyl, quinolyl, isoquinolyl, cinnolinyl, quinazolinyl, pyridopyridyl, benzoxazinyl, quinoxadinyl, chromenyl, chromanyl, isochromanyl, carbolinyl, thiazolyl, isoxazolyl, isoxazolonyl, isothiazolyl, triazolyl, oxadiazolyl, thiadiazolyl, benzodioxepinyl and pyridazyl. Preferred ranges of heteroaryl groups of the present invention are: C₂₋₃₀-heteroaryl, C₂₋₂₅-heteroaryl, C₂₋₂₀-heteroaryl, C₂₋₁₈-heteroaryl, C₂₋₁₅-heteroaryl, C₂₋₁₂-heteroaryl, C₂₋₁₀-heteroaryl, C₂₋₉-heteroaryl, C₂₋₈-heteroaryl and C₂₋₇-heteroaryl.

The term “heterocyclyl” refers to a saturated or partially unsaturated ring having three members of which at least one member is a N, O or S atom and which optionally contains one additional O atom or additional N atom; a saturated or partially unsaturated ring having four members of which at least one member is a N, O or S atom and which optionally contains one additional O atom or one or two additional N atoms; a saturated or partially unsaturated ring having five members of which at least one member is a N, O or S atom and which optionally contains one additional O atom or one, two or three additional N atoms; a saturated or partially unsaturated ring having six members of which one, two or three members are an N, O or S atom and which optionally contains one additional O atom or one, two or three additional N atoms; a saturated or partially unsaturated ring having seven members of which one, two or three members are an N, O or S atom and which optionally contains one additional O atom or one, two or three additional N atoms; a saturated or partially unsaturated ring having eight members of which one, two or three members are an N, O or S atom and which optionally contains one additional O atom or one, two or three additional N atoms; a saturated or partially unsaturated bicyclic ring having nine members of which at least one member is a N, O or S atom and which optionally contains one, two or three additional N atoms; or a saturated or partially unsaturated bicyclic ring having ten members of which one, two or three members are an N, O or S atom and which optionally contains one additional O atom or one, two or three additional N atoms. Preferably, heterocycles comprising peroxide groups are excluded from the definition of heterocyclyl. By way of non-limiting example, suitable heterocyclyl groups include pyrrolinyl, pyrrolidinyl, dioxolanyl, tetrahydrofuranyl, morpholinyl, imidazolinyl, imidazolidinyl, maleimidyl, pyrazolidinyl, piperidinyl, dihydropyranyl, succinimidyl, tetrahydropyranyl, thiopyranyl, tetrahydrothiopyranyl and piperazinyl. Preferred ranges of heterocyclyl groups of the present invention are: C₂₋₃₀-heterocyclyl, C₂₋₂₅-heterocyclyl, C₂₋₂₀-heterocyclyl, C₂₋₁₈-heterocyclyl, C₂₋₁₅-heterocyclyl, C₂₋₁₂-heterocyclyl, C₂₋₁₀-heterocyclyl, C₂₋₉-heterocyclyl, C₂₋₈-heterocyclyl and C₂₋₇-heterocyclyl.

As used herein, the term “alkaryl” and “alkylaryl” refer to an aryl group with an alkyl substituent. Binding is through the aryl group. Such groups have the number of carbon atoms as indicated. The alkyl and aryl moieties of such a group may be substituted as defined herein, with regard to the definitions of alkyl and aryl. The alkyl moiety may be straight or branched. Particularly preferred examples of alkaryl include tolyl, xylyl, butylphenyl, mesityl, ethyltolyl, methylindanyl, methylnaphthyl, methyltetrahydronaphthyl, ethylnaphthyl, dimethylnaphthyl, propylnaphthyl, butylnaphthyl, methylfluoryl and methylchrysyl. Again, preferred ranges of carbon atoms for alkaryl and alkylaryl groups of the present invention are: C₇₋₃₀, C₇₋₂₅, C₇₋₂₀, C₇₋₁₈, C₇₋₁₅, C₇₋₁₂, C₇₋₁₀ and C₇₋₉.

As used herein, the term “arylalkyl” refers to an alkyl group with an aryl substituent. Binding is through the alkyl group. Such groups have the number of carbon atoms as indicated. The aryl and alkyl moieties of such a group may be substituted as defined herein, with regard to the definitions of aryl and alkyl. The alkyl moiety may be straight or branched. Particularly preferred examples of arylalkyl include benzyl, methylbenzyl, ethylbenzyl, dimethylbenzyl, diethylbenzyl, methylethylbenzyl, methoxybenzyl, chlorobenzyl, dichlorobenzyl, trichlorobenzyl, phenethyl, phenylpropyl, diphenylpropyl, phenylbutyl, biphenylmethyl, fluorobenzyl, difluorobenzyl, trifluorobenzyl, phenyltolylmethyl, trifluoromethylbenzyl, bis(trifluoromethyl)benzyl, propylbenzyl, tolylmethyl, fluorophenethyl, fluorenylmethyl, methoxyphenethyl, dimethoxybenzyl, dichlorophenethyl, phenylethylbenzyl, isopropylbenzyl, diphenylmethyl, propylbenzyl, butylbenzyl, dimethylethylbenzyl, phenylpentyl, tetramethylbenzyl, phenylhexyl, dipropylbenzyl, triethylbenzyl, cyclohexylbenzyl, naphthylmethyl, diphenylethyl, triphenylmethyl and hexamethylbenzyl. Similarly, preferred ranges of carbon atoms for arylalkyl groups of the present invention are: C₇₋₃₀, C₇₋₂₅, C₇₋₂₀, C₇₋₁₈, C₇₋₁₅, C₇₋₁₂, C₇₋₁₀ and C₇₋₉.

The term “aminoalkyl” refers to an alkyl group with an amine substituent. Binding is through the alkyl group. Such groups have the number of carbon atoms as indicated above for “alkyl” groups. The alkyl moiety of such a group may be substituted as defined herein, with regard to the definition of alkyl. By way of non-limiting example, suitable aminoalkyl groups include aminomethyl, aminoethyl, aminopropyl, aminobutyl, aminopentyl and aminohexyl.

The term “aminoaryl” refers to an amine group with an aryl substituent. Binding is through the alkyl group. Such groups have the number of carbon atoms as indicated above for “aryl” groups. The aryl moiety of such a group may be substituted as defined herein, with regard to the definition of aryl.

As used herein, the term “acyl” refers to a group of general formula —C(O)—R, wherein R is selected from any one of the following groups: alkyl, alkenyl, alkynyl, aryl, heteroaryl, heterocyclyl, alkylaryl and arylalkyl.

With regard to one or more substituents which are referred to as being optionally substituted within a compound definition, for example, “alkaryl”, the substituent may be on either or both of the component moieties, e.g., on the alkyl and/or aryl moieties.

Reference to cyclic systems, e.g., aryl, heteroaryl, etc., contemplates monocyclic and polycyclic systems. Such systems comprise fused, non-fused and spiro conformations, such as bicyclooctyl, adamantyl, biphenyl and benzofuran.

The term “monosaccharide” means a sugar molecule having a chain of 3-10 carbon atoms in the form of an aldehyde (aldose) or ketone (ketose). Suitable monosaccharides for use in the invention include both naturally occurring and synthetic monosaccharides. Such monosaccharides include pentoses, such as xylose, arabinose, ribose, lyxose; methyl pentoses (6-deoxyhexoses), such as rhamnose and fructose; hexoses, such as allose, altrose, glucose, gulose, idose, mannose, galactose and talose. Preferred monosaccharides are hexoses.

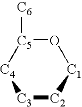

The monosaccharides may be attached to another monosaccharide group at the C₁, C₂, C₃, C₄, C₅ and C₆ position (shown above) to form a glycosyl bond and an oligosaccharide. Typically, a monosaccharide is attached to the C₃, C₄, C₅ and C₆ position through an oxygen atom attached to the C₁ carbon of another monosaccharide, which forms a glycosidic linkage and an oligosaccharide. Oligosaccharides that can be used in the present invention include disaccharides, trisaccharides, tetrasaccharides and pentasaccharides. However, in order to bind to AT, the oligosaccharide of the present invention is a pentasaccharide.

It will be appreciated that ionisable groups may exist in the neutral form shown in formulae herein, or may exist in charged form e.g. depending on pH. Thus a carboxylate group may be shown as —COOH, this formula is merely representative of the neutral carboxylate group, and other charged forms are encompassed by the invention (i.e. COO⁻).

Similarly, references herein to cationic and anionic groups should be taken to refer to the charge that is present on that group under physiological conditions e.g. where a sulphate group —OSO₃H is deprotonated to give the anionic —OSO₃⁻ group, this deprotonation is one that can occur at physiological pH. In addition where a carboxyl group —COOH is deprotonated to give the anionic —COO⁻ group, this deprotonation is one that can occur at physiological pH. Moreover, charged salts of the molecules of the invention are encompassed. Saccharide rings can exist in an open and closed form, while closed forms are shown herein, open forms are also encompassed by the invention.

Certain compounds of the invention exist in various regioisomeric, enantiomeric, tautomeric and diastereomeric forms. It will be understood that the invention comprehends the different regioisomers, enantiomers, tautomers and diastereomers in isolation from each other as well as mixtures.

The counter-ions, which compensate the charged forms of the compounds of the present invention, are pharmaceutically acceptable counter-ions such as hydrogen, or more preferably alkali or alkali-earth metals ions, which include sodium, calcium, magnesium and potassium.

Other ‘compound’ group definitions will be readily understandable by the skilled person based on the previous definitions and the usual conventions of nomenclature.

It will be appreciated that any optional feature that has been described above in relation to any one aspect of the invention may also be applicable to any other aspect of the invention.

### GENERAL PROCEDURES

The present invention will now be described in more detail, by way of the following non-limiting methods that can be used for synthesising the compounds of the present invention. The skilled person will, however, appreciate that these methods merely illustrate the present invention and in no way restrict its scope.

### General Preparatory Synthetic Scheme A

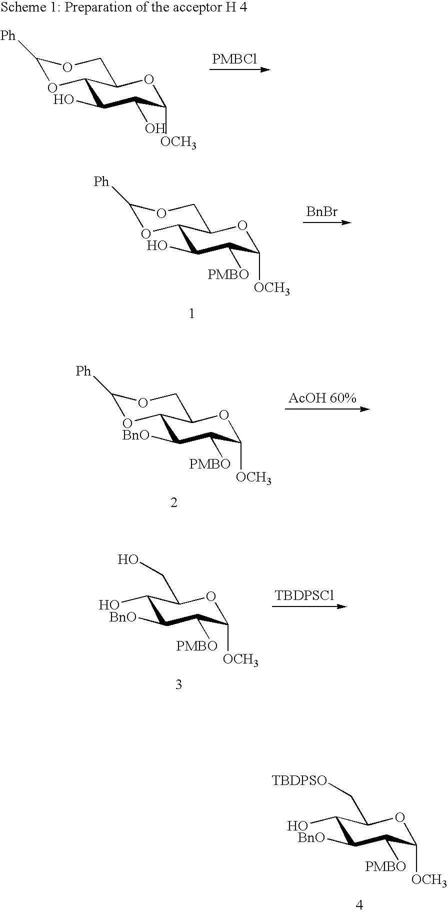

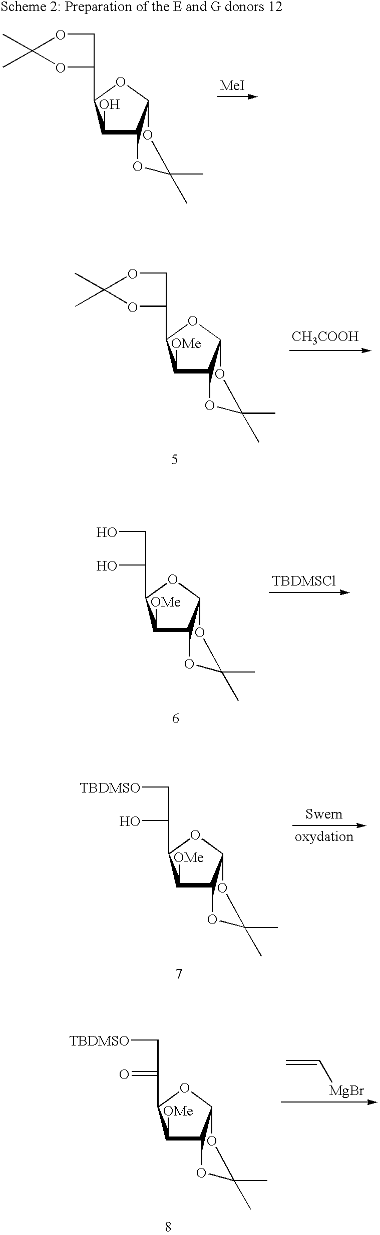

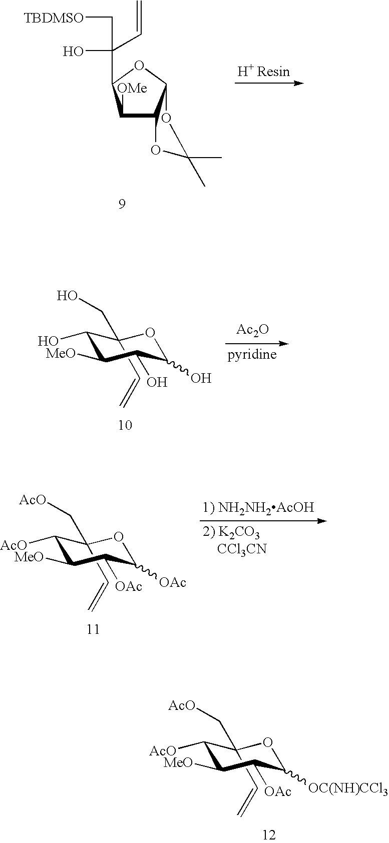

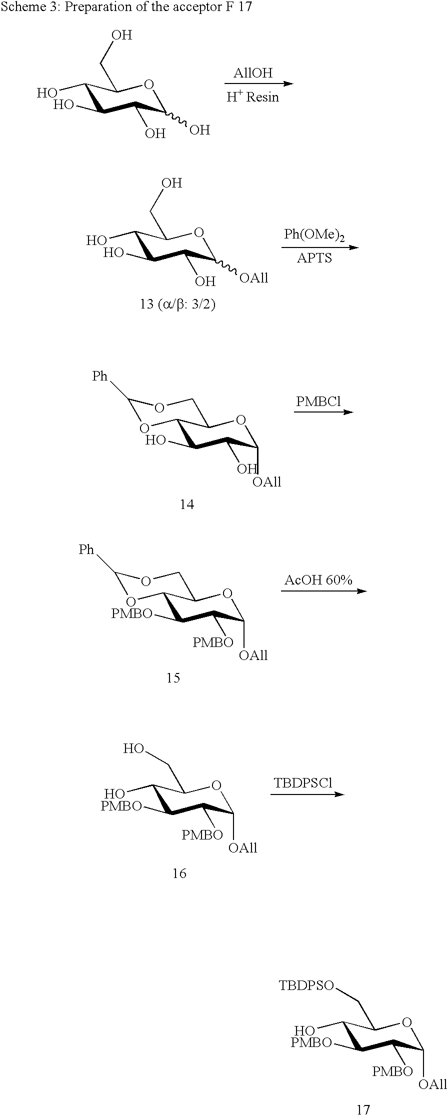

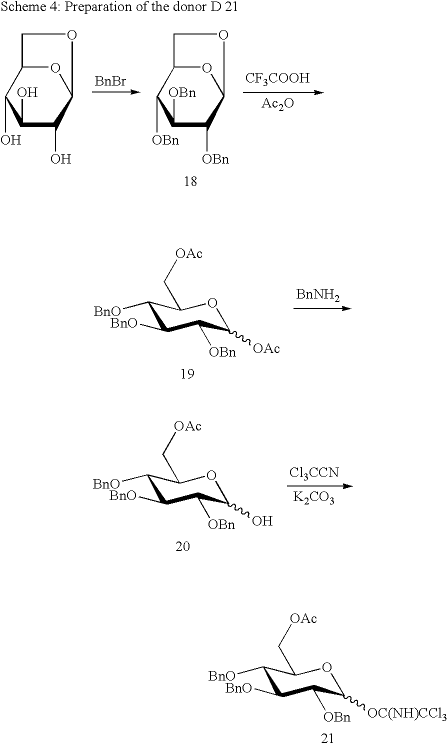

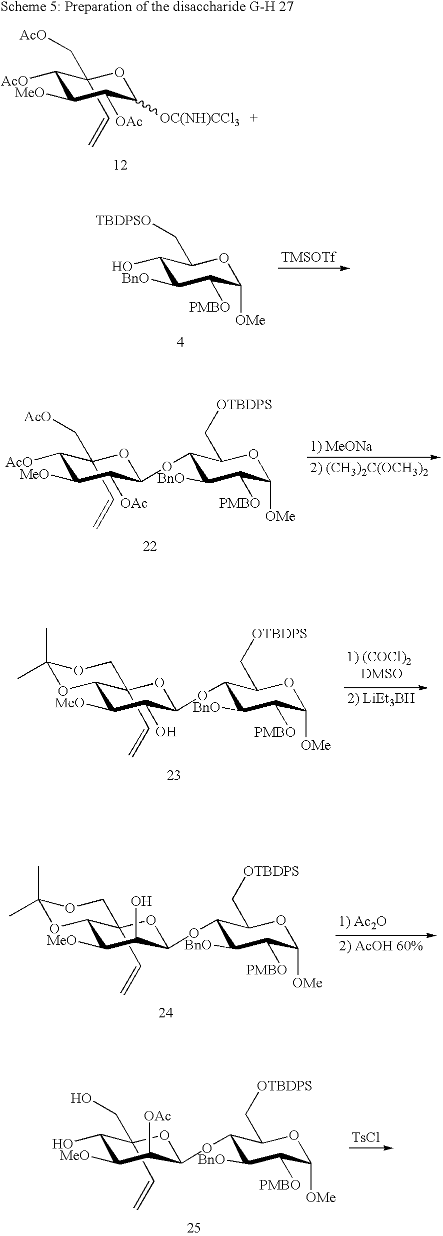

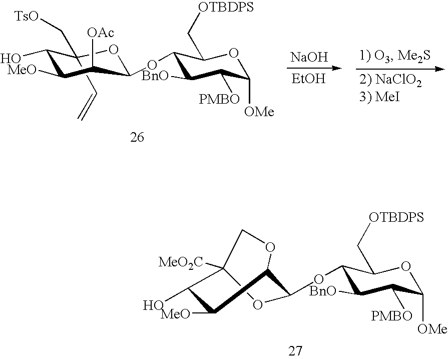

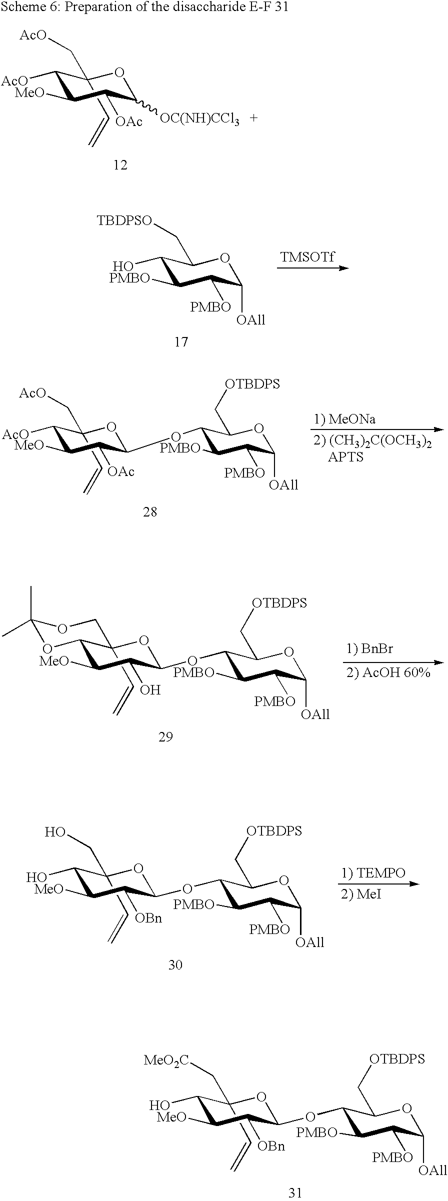

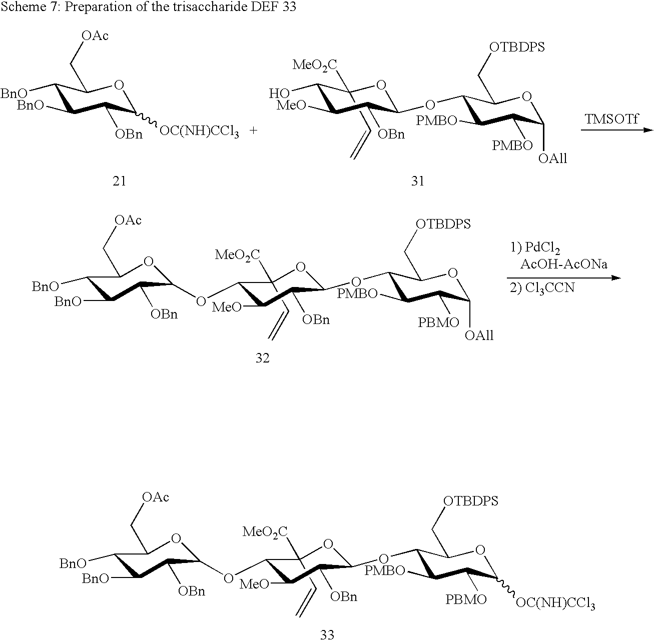

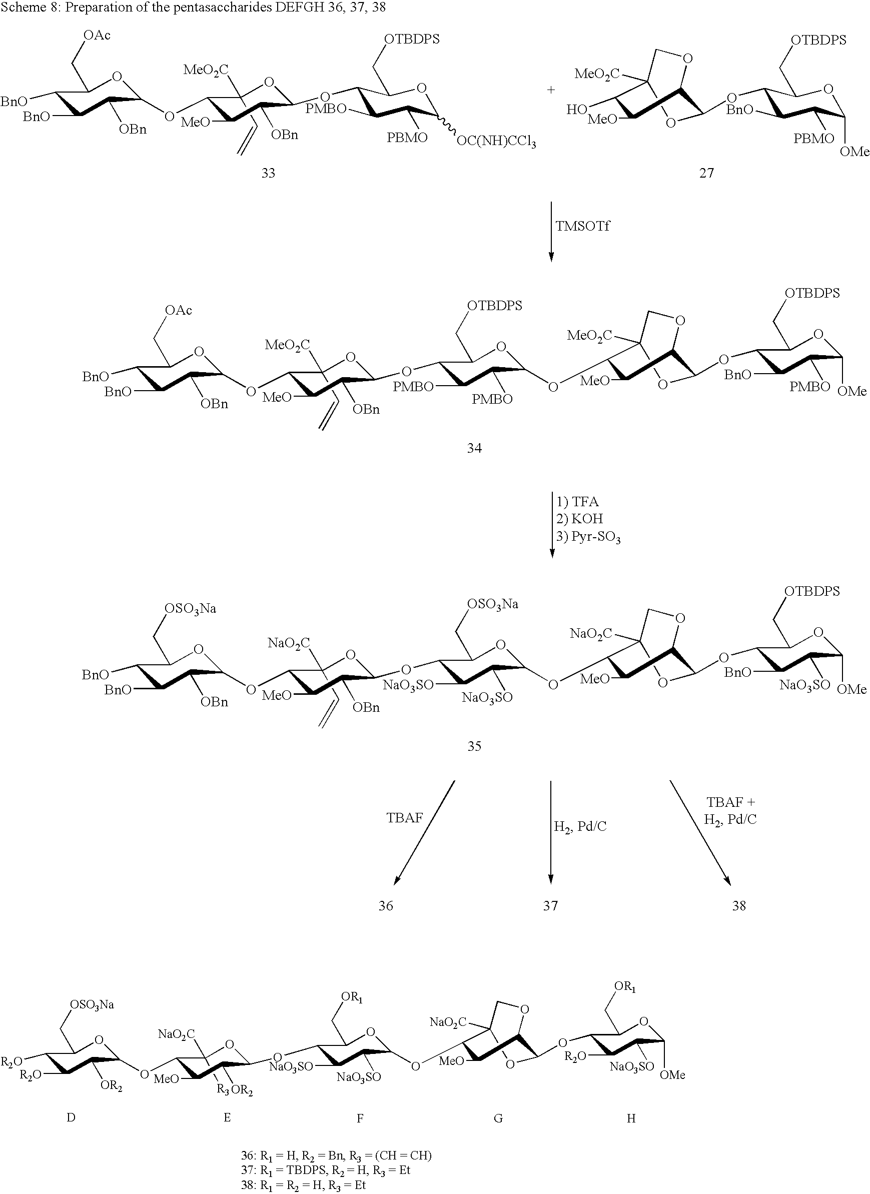

### Method A: Desilylation

Ammonium fluoride (40 molar equivalents) was added to a solution of pentasaccharide (1 molar equivalent) in methanol (70 L/mol). After stirring at room temperature for 72 h, chromatography on a Sephadex LH-20 column (20 L/mmol) equilibrated with CH₂Cl₂/methanol/water (50:50:1) gave the desilylated product.

### Method B: Hydrogenolysis

A solution of pentasaccharide (1 molar equivalent) in 13:20 tort-butanol/water (250 L/mol) was stirred under hydrogen in the presence of Pd/C catalyst (10%, 2 weight equivalents) for 24 h and filtered through Celite® 45.

### Method C: Alkylation

NaH 60%/oil (18 molar equivalents) was added to a solution of pentasaccharide (1 molar equivalent) in DMF (70 L/mol) at 0° C. and the mixture was stirred for 15 minutes. After this time, the alkylating agent (18 molar equivalents) was added and the solution was stirred at room temperature for 5 h. The resultant solution was then neutralised with ethanol, and directly poured onto a Sephadex LH-20 column (20 L/mmol) equilibrated with CH₂Cl₂/methanol/water (50:50:1) to give the alkylated product.

### Method D: Acylation

An acyl chloride (10 molar equivalents) was added to a solution of pentasaccharide (1 molar equivalent) in pyridine (70 L/mol) and DMF (70 L/mol) at 0° C. The mixture was stirred for 16 h at room temperature and directly poured onto a Sephadex LH-20 column (20 L/mmol) equilibrated with CH₂Cl₂/methanol/water (50:50:1) to give the acylated product.

### Intermediate Products

The monosaccharides 4 and 21 were prepared according to procedures well known in the art. In addition, the monosaccharide donor 12 was prepared according to the procedure described in U.S. Pat. No. 6,670,338 and Chem. Eur. J., 2001, 7(22), 4821.

### Preparation of the Disaccharide GH 22

A mixture of 4 (92.1 mg, 0.143 mmol), 12 (84.1 mg, 0.172 mmol) and 4 Å molecular sieves (220 mg) in toluene (2 mL) was stirred at room temperature for 30 minutes. The suspension was cooled at −40° C. and a 0.14 M solution of TMSOTf in toluene (0.2 ml, 0.17 eq/imidate) was added. The reaction mixture was then stirred for 90 minutes and the temperature was allowed to rise gradually to −20° C. After this time, the reaction mixture was neutralized with triethylamine, diluted with CH₂Cl₂, filtered through Celite® and concentrated.

Preparative TLC on silica gel (Toluene/AcOEt: 80/20+1% Et₃N) gave compound 22 (133.6 mg, 96%), which had the following properties: TLC: Rf=0.22, silica gel, toluene/AcOEt: 90/10 v/v; chemical shifts of the anomeric protons: 5.14 and 4.70 ppm; and MS (ESI⁺): m/z 993.4 [M+Na]⁺.

Disaccharide 22 was then transformed into disaccharide 27 according to the methods described in Chem. Eur. J., 2001, 7(22), 4821.

### Preparation of the Disaccharide EF 28

A mixture of 17 (1.025 g, 1.467 mmol), 12 (864 mg, 1.76 mmol) and 4 Å molecular sieves (2.2 g) in toluene (20 mL) was stirred at room temperature for 30 minutes. The suspension was cooled at −40° C. and a 0.29 M solution of TMSOTf in toluene (1 ml, 0.17 eq/imidate) was added. The reaction mixture was then stirred for 90 minutes and the temperature was allowed to rise gradually to −20° C. After this time, the reaction mixture was neutralized with triethylamine, diluted with CH₂Cl₂, filtered through Celite® and concentrated.

Flash column chromatography on silica gel (CH₂Cl₂/AcOEt: 93/7) gave compound 28 (1.36 g, 75%), which had the following properties: TLC: Rf=0.36, silica gel, toluene/AcOEt: 80/20 v/v; chemical shifts of the anomeric protons: 5.15 and 4.90 ppm; and MS (ESI⁺): m/z 1049.4 [M+Na]⁺.

Disaccharide 28 was then transformed into the disaccharide 31 following the methods described in Chem. Eur. J., 2001, 7(22), 4821.

### Preparation of the Trisaccharide DEF 32

A mixture of 31 (402 mg, 0.367 mmol), 21 (281 mg, 0.441 mmol) and 4 Å molecular sieves (1.0 g) in toluene (8 mL) was stirred at room temperature for 30 minutes. The suspension was cooled at −40° C. and a 0.29 M solution of TMSOTf in toluene (250 μl, 0.17 eq/imidate) was added. The reaction mixture was then stirred for 90 minutes and the temperature was allowed to rise gradually to −20° C. After this time, the reaction mixture was neutralized with triethylamine, diluted with CH₂Cl₂, filtered through Celite® and concentrated.

Flash column chromatography on silica gel (CH₂Cl₂/AcOEt: 95/5) gave compound 32 (436 mg, 71%), which had the following properties: TLC: Rf=0.26, silica gel, toluene/AcOEt: 80/20 v/v; chemical shifts of the anomeric protons: 5.16, 5.08 and 4.88 ppm; and MS (ESI⁺): m/z 1592.7 [M+Na]⁺.

Trisaccharide 32 was then transformed into the trisaccharide 33 according to the methods described in Chem. Eur. J., 2001, 7(22), 4821.

### Preparation of Protected Pentasaccharide DEFGH 34

A mixture of 33 (189 mg, 0.113 mmol), 27 (88 mg, 0.094 mmol) and 4 Å molecular sieves (500 mg) in toluene (4 mL) was stirred at room temperature for 30 minutes. The suspension was cooled at −40° C. and a 0.29 M solution of TMSOTf in toluene (64 μl, 0.17 eq/imidate) was added. The reaction mixture was then stirred for 90 minutes and the temperature was allowed to rise gradually to −20° C. After this time, the reaction mixture was then neutralized with triethylamine, diluted with CH₂Cl₂, filtered through Celite® and concentrated.

Flash column chromatography on silica gel (CH₂Cl₂/AcOEt: 95/5) gave compound 34 (168 mg, 73%), which had the following properties: TLC: Rf=0.33, silica gel, CH₂Cl₂/AcOEt: 90/10 v/v; chemical shifts of the anomeric protons: 5.50, 5.28, 5.16, 4.98 and 4.72 ppm; MS (ESI⁺): m/z 1592.7 [M+Na]⁺.

### Preparation of Pentasaccharide DEFGH 35

Pentasaccharide 34 (115 mg, 50 μmol) was dissolved at 0° C. in 18.4 ml of a mixture CH₂Cl₂/TFA (99/1). The solution was stirred at room temperature during 12 h and diluted with CH₂Cl₂.

After washing with aqueous saturated NaHCO₃ solution, the organic layer was dried on MgSO₄, concentrated and purified by chromatography on silica gel (CH₂Cl₂/MeOH: 95/5) to give 76 mg of an intermediate pentasaccharide which was dissolved in 5.4 ml of a mixture THF/MeOH (2/1). Then, 1.7 ml of a 2 M aqueous KOH solution were added dropwise at 0° C. and the mixture was stirred 2 h at room temperature. After stirring, the reaction mixture was acidified with ion-exchange resin Dowex 50WX8-200, filtered and concentrated to dryness.

The resultant pentasaccharide was dissolved in 7.6 ml of dry pyridine and sulphurtrioxide pyridine complex (181 mg, 1 mmol) was added. The mixture was heated at 55° C. with protection of light for 18 h.

After cooling to 0° C., the solution was neutralized with MeOH and an aqueous saturated NaHCO₃ solution. The reaction mixture was directly poured onto Sephadex LH20 (dichloromethane/methanol: 1/1+water 1%) to give the O-sulfonated pentasaccharide 35 (60 mg, 53%), which had the following properties: chemical shifts of the anomeric protons: 5.47, 5.31, 5.16, 4.71 and 4.67 ppm; MS (ESI⁻): chemical mass=2316.37; experimental mass=2318.3.

### Preparation of Pentasaccharide DEFGH 36

Pentasaccharide 35 (53 mg, 21.6 μmol) was desilylated according to ‘Method A: Desilylation’ to give pentasaccharide 36 (37 mg, 86%), which had the following properties: chemical shifts of the anomeric protons: 5.39, 5.29, 5.17, 4.68 and 4.66 ppm; and MS (ESI⁻): chemical mass=1838.25; experimental mass=1839.5.

### Preparation of Pentasaccharide DEFGH 37

Pentasaccharide 35 (42 mg, 17.1 μmol) was hydrogenolysed according to ‘Method B: Hydrogenolysis’ to give pentasaccharide 37 (35.7 mg, 85%), which had the following properties: chemical shifts of the anomeric protons: 5.41, 5.29, 5.16, 4.67 and 4.65 ppm; and MS (ESI⁻): chemical mass=1728.33; experimental mass=1729.4.

### Preparation of Pentasaccharide DEFGH 38

Pentasaccharide 36 (37 mg, 18.6 μmol) was hydrogenolysed according to ‘Method B: Hydrogenolysis’ to give pentasaccharide 38 (21.5 mg, 83%), which had the following properties: chemical shifts of the anomeric protons: 5.31, 5.22, 5.03, 4.69 and 4.61 ppm; and MS (ESI⁻): chemical mass=1252.09; experimental mass=1253.1.

### General Preparatory Synthetic Scheme B

### Method E: General Method for O-Alkylation

To a dry round-bottom flask was introduced 1,6-β-anhydroglucopyranose in anhydrous DMF (0.3 M) followed by NaH (7 eq.). The solution was stirred for 30 min at 0° C. before RX (X═Cl or Br, 8 eq.) was added dropwise. The reaction was stirred at 0° C. overnight and MeOH was added to quench the excess of NaH. The reaction was then stirred for 30 min and subsequently diluted with ethyl acetate. The organic layer was successively washed with a NaCl saturated solution, water and a saturated aqueous solution of NaHCO₃. The organic layer was dried over MgSO₄, filtered and concentrated under reduced pressure. If necessary, purification was performed using silica gel column chromatography to give O-alkylated-1,6-β-anhydroglucopyranose.

### Method F: General Method for Acetolysis

In a dry round-bottom flask O-alkylated-1,6-β-anhydroglucopyranose was dissolved in a mixture of acetic anhydride (0.1 M) and TFA (11 eq.). The reaction mixture was stirred for 1.5 h at room temperature and solvents were removed under reduced pressure followed by co-evaporation with toluene.

### Method G: General Method for Selective Deacetylation

In a dry round-bottom flask, the saccharide to be deacetelylated was introduced to a mixture of THF/MeOH (7/3, 0.03 M) and the solution was cooled to 0° C. After stirring for 15 min, the solution was bubbled with a gentle flow of ammonia for 2 h (TLC showed disappearance of the starting material). The reaction mixture was then purged with nitrogen for 20 min and concentrated to dryness under reduced pressure. The crude product was directly used in the next step without any further purification.

### Method H: General Method for Trichloroacetimidate Formation

In a dry round-bottom flask, a saccharide was dissolved in dry dichloromethane (0.1 M), followed by addition of CCl₃CN (9 eq.) and K₂CO₃ (2.7 eq.) previously activated at 400° C. overnight. After stirring at room temperature overnight, the reaction mixture was diluted in dichloromethane, filtered through a pad of Celite®, washed and the filtrate was concentrated to dryness. The resultant residue was purified by chromatography on silica gel to afford the desired trichloroacetimidate.

### Method I: General Method for Coupling

To a dry round-bottom flask was added under nitrogen both acceptor and donor in a mixture of dichloromethane/diethyl ether (1/1 or 1:2, 0.1 eq./acceptor) containing 4 A molecular sieves (1 weight eq./acceptor). After stirring for 1 h, temperature was cooled down to −20° C. and trimethylsilyl trifluoromethanesulfonate or tert-butyldimethylsilyl trifluoromethanesulfonate (0.2 eq. vs donor) was added. After an additional 3 h, TLC analysis indicated that the reaction went to completion. The excess of reagent was neutralized with triethylamine until pH 7 and the solution was filtered through a pad of Celite®. The filtrate was then evaporated to dryness under reduced pressure and purified using a Sephadex LH-20 gel column (dichloromethane/ethanol: 1/1) or purified by silica chromatography to afford the desired product.

### Method J: General Method for Saponification

Initially, a pentasaccharide that was to be saponified was dissolved in a THF/MeOH mixture (2/1, 0.01M). The solution was cooled to 0° C. and 2M KOH (90 eq.) was added. Stirring was maintained until completion of the reaction, wherein the reaction temperature was allowed to increase to room temperature. The reaction was then acidified by addition of Dowex® 50WX8-200 until pH 4-5. Purification using a sephadex LH-20 (CH₂Cl₂/EtOH: 1/1) gave the saponified product.

### Method K: General Method for Sulphation

Initially, a pentasaccharide that was to be sulphated was dissolved in dry pyridine (0.015M). Sulphur trioxide pyridine complex (5 eq. per OH to be sulphated) was added. The mixture was protected from light, heated at 80° C. for 3 h and then cooled to 0° C. Methanol (10 eq./sulphur trioxide pyridine complex eq.) was added dropwise, followed by addition of a saturated NaHCO₃ aqueous solution (to reach pH 9). After stirring overnight at room temperature, the mixture was filtered and the filtrate was directly applied to the top of a sephadex LH-20 column eluted with dimethylformide. Fractions containing the product were pooled together and the solvent was concentrated under vacuum to afford the sulphated pentasaccharide.

### Method L: General Method for Desilylation

Initially, a pentasaccharide that was to be desilylated was dissolved in dry methanol (0.02M) and ammonium fluoride (20 eq.) was added. The mixture was stirred overnight at 50° C. and then cooled to 0° C. A saturated NaHCO₃ aqueous solution was added to reach pH 9. After filtration of the mixture, the filtrate was directly applied to the top of a sephadex LH-20 column eluted with dimethylformide. Fractions containing the product were pooled together and the solvent was concentrated under vacuum to afford the desired pentasaccharide.

### Method M: Hydrogenolysis

In a dry round bottom flask, the oligosaccharide that was to be reduced was mixed with Pd/C or Pd(OH)₂ (10 mg, 1 weight eq.) and tert-BuOH/H₂O (1:1, 10 mg/mL). The reaction mixture was cooled to 0° C., purged with hydrogen and stirred under an atmosphere of hydrogen. The reaction mixture was filtered and lyophylised to afford a white amorphous solid.

### Preparation of Monosaccharides

### Preparation 1: Synthesis of Monosaccharides 51, 52, 53 and 54

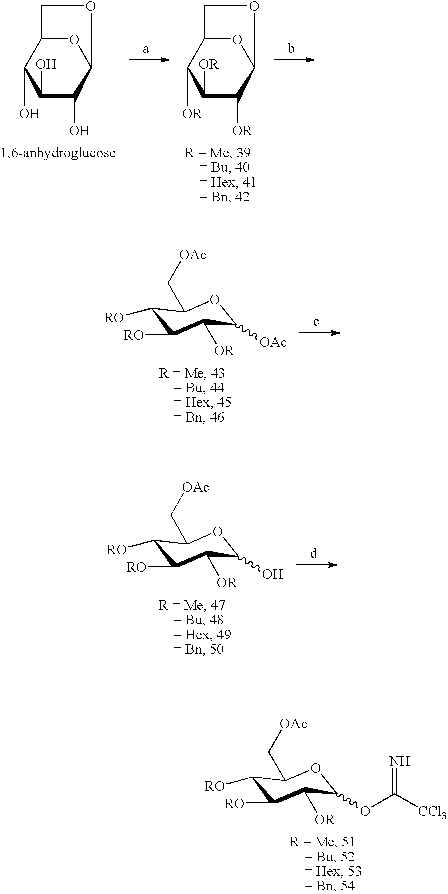

### Synthesis of 2,3,4-tri-O-methyl-6-O-acetyl-D-glucopyranosyl trichloroacetimidate 51

### Step 1.a: Synthesis of 2,3,4-tri-O-methyl-1,6-anhydro-β-glucopyranose 39

O-alkylation of 1,6-anhydro-β-D-glucopyranose (4 g, 25 mmol) was performed as described in Method E, which gave crude compound 39 (6 g) that was used in the next step without any further purification.

¹H NMR (400 MHz, CDCl₃, ppm), δ=5.49 (s, 1H, H-1), 4.64 (d, 1H, J_{5,6}=5.8 Hz, H-5); 3.93 (d, 1H, J_{6a,6b}=7.2 Hz, H-6); 3.74 (dd, 1H, J_{6a,6b}=7.2 Hz, J_{5,6}=5.8 Hz, H-6); 3.49, 3.48 and 3.46 (3s, 9H, OMe); 3.34 (sl, 1H, H-3); 3.15 (sl, 1H, H-2); 3.11 (sl, 1H, H-4)

### Step 1.b: Synthesis of 2,3,4-tri-O-methyl-1,6-di-O-acetyl-α,β-D-glucopyranose 43

Acetolysis of 1,6-anhydro-2,3,4-tri-O-methyl-β-D-glucopyranose 39 (5 g, 24.7 mmol) was performed as described in Method F, which gave crude compound 43 (5.82 g, quantitative yield, α/β: 83/17) that was used in the next step without any further purification.

¹H NMR (400 MHz, CDCl₃, ppm), δ=6.30 (s, 0.83H, H-1α), 5.49 (d, 0.17H, J_{1,2}=8.6 Hz, H-1β); 3.66, 3.55 and 3.48 (3s, 9H, OMe); 2.16 and 2.10 (s, 6H, CH₃—Ac).

### Step 1.c: Synthesis of 2,3,4-tri-O-methyl-6-O-acetyl-α,β-D-glucopyranose 47

Selective hydrolysis of 1,6-di-O-acetyl-2,3,4-tri-O-methyl-β-D-glucopyranose 43 (1.6 g, 5.26 mmol) was performed as described in Method G, which gave crude compound 47 (1.01 g, 74%, α/β: 63/37) that was used in the next step without any further purification.

¹H NMR (400 MHz, CDCl₃, ppm), δ=5.34 (sl, 0.63H, H-1α), 4.61 (d, 0.37H, J=7.5 Hz, H-1β), 4.41 to 4.13 (m, 2H, H-6); 3.65, 3.54, 3.53 (s, 9H, OMe); 2.11 and 2.12 (s, 3H, OAc).

### Step 1d: Synthesis of 2,3,4-tri-O-methyl-6-O-acetyl-α,β-D-glucopyranosyl trichloroacetimidate 51

Trichloroacetimidate formation of 6-O-acetyl-2,3,4-tri-O-methyl-α,β-D-glucopyranose 47 (0.719 g, 2.3 mmol) was performed as described in Method H, which gave, after purification, compound 51 (α/β: 19/81, 0.935 g).

¹H NMR (400 MHz, CDCl₃, ppm), δ=8.60 (s, 1H, NH), 6.48 (d, 1H, J=3.5 Hz, H-1α, 19%), 5.66 (dd, 1H, J=2.2, 5.5 Hz, H-1β, 81%), 3.70 (s, 3H, OMe), 3.60 (s, 3H, OMe), 3.50 (s, 3H, OMe), 2.10 (s, 3H, OAc).

Monosaccharides 52, 53 and 54 were prepared by following same procedures that have been outlined above for the synthesis of 2,3,4-tri-O-methyl, 6-O-acetyl-D-glucopyranosyl trichloroacetimidate 51.

### Synthesis of 2,3,4-tri-O-butyl-6-O-acetyl-α,β-D-glucopyranosyl trichloroacetimidate 52

Trichloroacetimidate formation of 6-O-acetyl-2,3,4-tri-O-butyl-α,β-D-glucopyranose 48 (1.15 g, 2.9 mmol) was performed as described in Method H, which gave, after purification, compound 52 (1.36 g, 87%, α/β: 2/1).

¹H NMR (400 MHz, CDCl₃, ppm), δ=8.62 (s, 0.1H, NHβ), 8.56 (s, 0.19H, NHα), 6.45 (d, 1H, J=3.5 Hz, H-1α, 19%), 5.64 (dd, 1H, J=2.0 Hz, 8.0 Hz, H-1β, 81%), 2.07 (s, 3H, OAc).

### Synthesis of 2,3,4-tri-O-hexyl-6-O-acetyl-α,β-D-glucopyranosyl trichloroacetimidate 53

Trichloroacetimidate formation of 6-O-acetyl-2,3,4-tri-O-hexyl-α,β-D-glucopyranose 49 (1.15 g, 2.34 mmol) was performed as described in Method H, which gave, after purification, compound 53 (α/β: 24/76, 1.34 g, 92%).

¹H NMR (400 MHz, CDCl₃, ppm), δ=8.65 (s, 0.24H, NHα), 8.57 (s, 0.76H, NHβ, 6.47 (d, 0.24H, J_{1,2}=3.6 Hz, H-1α), 5.67 (dd, 0.76H, J_{1,2}=5.9 Hz and J_{1,3}=2.1 Hz H-1β); 4.37-4.2 (m, 2H); 4.2-4.1 (m, 1.5H); 2.08 and 2.07 (s, 3H, CH₃—OAc); 1.68-1.45 (m, 6H, O—CH₂—CH₂—CH₂—CH₂—CH₂—CH₃); 1.43-1.21 (m, 18H, O—CH₂—CH₂—CH₂—CH₂—CH₂—CH₃); 0.97-0.84 (m, 9H, O—CH₂—CH₂—CH₂—CH₂—CH₂—CH₃).

### Synthesis of 2,3,4-tri-O-benzyl-6-O-acetyl-α,β-D-glucopyranosyl trichloroacetimidate 54

Trichloroacetimidate formation of 2,3,4-tri-O-benzyl-6-O-acetyl-α,β-D-glucopyranose 50 (0.71 g, 1.44 mmol) was performed as described in Method H and gave after purification compound 54 (214 mg isomer α, 646 mg isomer β, α/β=3/7, 94%).

### Isomer α:

¹H NMR (400 MHz, CDCl₃, ppm), δ=8.63 (s, 1H, N—H); 7.4-7.27 (m, 15H, arom.); 6.49 (d, 1H, J_{1,2}=3.4 Hz, H-1α), 5.01 (d, 1H, J=10.7 Hz, CH-Ph); 4.91 and 4.86 (q_{AB}, 2H, J=10.9 Hz, CH-Ph); 4.77 and 4.71 (q_{AB}, 2H, J=11.75 Hz, CH-Ph); 4.61 (d, 1H, J=10.7 Hz, CH-Ph); 4.24 (m, 2H, H-6); 4.14-4.03 (m, 2H, H-4 and H-5); 3.77 (dd, 1H, J_{1,2}=3.4 Hz and J_{2,3}=9.6 Hz); 3.62 (t, 1H, J_{3,4}=J_{2,3}=9.6 Hz); 2.04 (s, 3H, CH₃—OAc).

### Isomer β:

¹H NMR (400 MHz, CDCl₃, ppm), δ=8.7 (s, 1H, N—H); 7.4-7.25 (m, 15H, arom.); 5.84 (d, 1H, J_{1,2}=7.62 Hz, H-1β), 4.97 (d, 1H, J=10.9 Hz, CH-Ph); 4.95 (d, 1H, J=10.9 Hz, CH-Ph); 4.87 and 4.78 (q_{AB}, 2H, J=10.9 Hz, CH-Ph); 4.83 (d, 1H, J=10.9 Hz, CH-Ph); 4.61 (d, 1H, J=10.9 Hz, CH-Ph); 3.38-4.24 (m, 2H, H-6); 3.85-3.63 (m, 4H, H-2, H-3, H-4 and H-5); 2.03 (s, 3H, CH₃—OAc).

### Preparation 2: Synthesis of Monosaccharides 75, 76, 77, 78 and 79

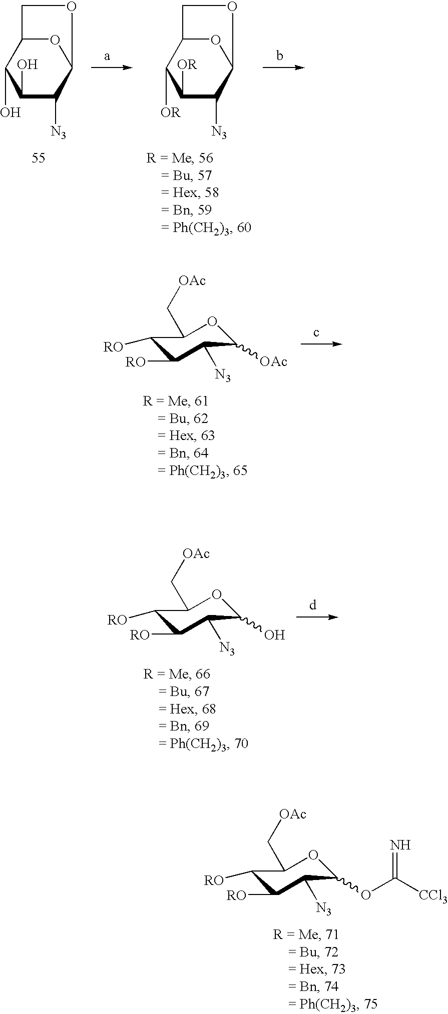

### Synthesis of 1,6-O-acetyl-2-azido-2-deoxy-3,4-di-O-methyl-α,β-D-glucopyranose trichloroacetimidate 71

### Step 2.a: Synthesis of 1,6-anhydro-2-azido-2-deoxy-3,4-di-O-methyl-β-D-glucopyranose 56

O-alkylation of 1,6-anhydro-2-azido-2-deoxy-β-D-glucopyranose 55 (3 g, 16.03 mmol) was performed as described in Method E, which gave crude compound 56 (4 g) that was directly used in the following step without any further purification.

### Step 2.b: Synthesis of 1,6-di-O-acetyl-2-azido-2-deoxy-3,4-di-O-methyl-α,β-D-glucopyranose 61

Acetolysis of 1,6-anhydro-2-azido-2-deoxy-3,4-di-O-methyl-β-D-glucopyranose 56 (3.45 g, 16.03 mmol) was performed as described in Method F, which gave crude compound 61 (α/β=89/11, 4.3 g) that was used in the following step without any further purification.

¹H NMR (400 MHz, CDCl₃, ppm), δ=6.17 (d, 0.89H, J_{1,2}=3.5 Hz, H-1α), 5.43 (d, 0.11H, J_{1,2}=8.7 Hz, H-1β), 4.29-4.25 (m, 2H, H-6); 3.81 (m, 1H, H-5); 3.72 (s, 3H, OMe), 3.57 (s, 3H, OMe), 3.46 (m, 1H, H-2); 3.24 (m, 1H, H-3); 2.17 (s, 3H, OAc), 2.11 (s, 3H, OAc).

### Step 2.c: Synthesis of 6-O-acetyl-2-azido-2-deoxy-3,4-di-O-methyl-α,β-D-glucopyranose 66

Selective anomeric acetate hydrolysis of 1,6-di-O-acetyl-2-azido-2-deoxy-3,4-di-O-methyl-α,β-D-glucopyranose 61 (5.09 g, 16.03 mmol) was performed as described in Method G, which gave crude compound 66 (5.65 g) that was directly used in the following step without any further purification.

### Step 2.d: Synthesis of 6-O-acetyl-2-azido-2-deoxy-3,4-di-O-methyl-α,β-D-glucopyranose trichloroacetimidate 71

Trichloroacetimidate formation of 6-O-acetyl-2-azido-2-deoxy-3,4-di-O-methyl-α,β-D-glucopyranose 66 (4.41 g, 16.03 mmol) was performed as described in Method H, which gave, after purification, compound 71 (α/β: 32/68, 5.47 g, 81% over 4 steps).

¹H NMR (400 MHz, CDCl₃, ppm), δ=8.79 (s, 1H, NH), 6.34 (d, 0.32H, J_{1,2}=3.5 Hz, H-1α), 5.58 (d, 0.68H, J_{1,2}=8.7 Hz, H-1β), 4.39-4.22 (m, 2H, H-6); 3.70 (s, 3H, OMe), 3.56 (s, 3H, OMe), 3.33-3.16 (m, 1H, H-2); 2.10 (s, 3H, OAc).

Monosaccharides 72, 73, 74 and 75 were prepared following the same procedures that were used for the synthesis of 6-O-acetyl-2-azido-2-deoxy-3,4-di-O-methyl-α,β-D-glucopyranose trichloroacetimidate 71.

### Synthesis of 6-O-acetyl-2-azido-2-deoxy-3,4-di-O-butyl-α,β-D-glucopyranose trichloroacetimidate 72

Trichloroacetimidate formation of 6-O-acetyl-2-azido-2-deoxy-3,4-di-O-butyl-α,β-D-glucopyranose 67 (1.8 g, 5.01 mmol) was performed as described in Method H, which gave, after purification, compound 72 (α/β: 43/57, 2.52 g, 84% over 4 steps).

¹H NMR (400 MHz, CDCl₃, ppm), δ=8.71 (s, 1H, NH), 6.35 (d, 0.43H, J_{1,2}=3.4 Hz, H-1α), 5.54 (d, 0.57H, J_{1,2}=8.4 Hz, H-1β), 2.08 (s, 3H, OAc).

### Synthesis of 6-O-acetyl-2-azido-2-deoxy-3,4-di-O-hexyl-α,β-D-glucopyranose trichloroacetimidate 73

Trichloroacetimidate formation of 6-O-acetyl-2-azido-2-deoxy-3,4-di-O-hexyl-α,β-D-glucopyranose 68 (2.34 g, 5.63 mmol) was performed as described in Method H, which gave, after purification, compound 73 (α/β: 37/63, 2.29 g, 73% over 4 steps).

¹H NMR (400 MHz, CDCl₃, ppm), δ=8.70 (s, 1H, NH), 6.34 (d, 0.37H, J_{1,2}=3.4 Hz, H-1α), 5.56 (d, 0.63H, J_{1,2}=8.5 Hz, H-1β), 2.08 (s, 3H, OAc).

### Synthesis of 6-O-acetyl-2-azido-2-deoxy-3,4-di-O-benzyl-α,β-D-glucopyranose trichloroacetimidate 74

Trichloroacetimidate formation of 6-O-acetyl-2-azido-2-deoxy-3,4-di-O-benzyl-α,β-D-glucopyranose 69 (1.16 g, 2.72 mmol) was performed as described in Method H, which gave, after purification, compound 74 (α/β=7/3, 1.31 g, 84% over 4 steps).

### Isomer α

¹H NMR (400 MHz, CDCl₃, ppm), δ=8.77 (s, 1H, NH), 6.43 (d, 1H, J_{1,2}=3.6 Hz, H-1α), 2.04 (s, 3H, OAc).

### Isomer β

¹H NMR (400 MHz, CDCl₃, ppm), δ=8.75 (s, 1H, NH), 5.64 (d, 0.63H, J_{1,2}=8.0 Hz, H-1β), 2.04 (s, 3H, OAc).

### Synthesis of 6-O-acetyl-2-azido-2-deoxy-3,4-di-O-(3-phenylpropyl)-α,β-D-glucopyranose trichloroacetimidate 75

Trichloroacetimidate formation of 6-O-acetyl-2-azido-2-deoxy-3,4-di-O-(3-phenylpropyl)-α,β-D-glucopyranose 70 (1.18 g, 2.45 mmol) was performed as described in Method H, which gave, after purification, compound 75 (α/β: 27/73, 1.02 g, 66% over 4 steps).

¹H NMR (400 MHz, CDCl₃, ppm), δ=8.74 (s, 1H, NH), 6.38 (d, 0.27H, J=3.4 Hz, H-1α, 0.27), 5.59 (d, 0.73H, J=8.5 Hz, H-1,0, 0.73), 2.07 (s, 3H, OAc).

### Preparation 3: Synthesis of Monosaccharides 79 and 80

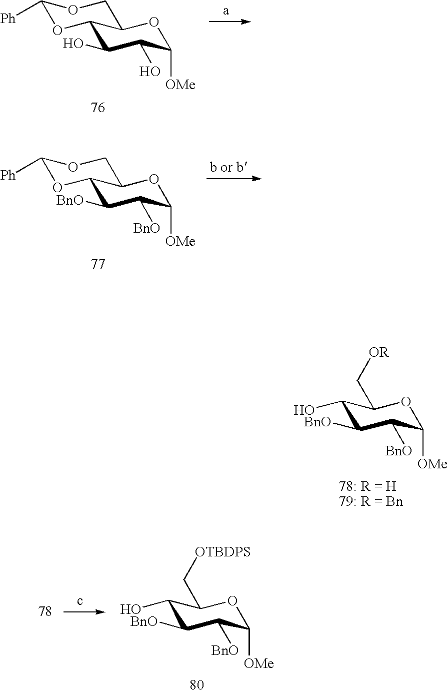

### Step 3.a: Synthesis of methyl 4,6-benzylidene-2,3-di-O-benzyl-α-D-glucopyranoside 77

In a 1 L round-bottom flask, under an atmosphere of Ar, at 0° C., compound 76 (50 g, 172 mmol) in dry DMF (100 mL) was added to NaH (60% in oil) (20.64 g, 516 mmol, 3 eq.) in suspension in dry DMF (400 mL). After stirring for 1 h at 0° C., DMF (200 mL) was added (precipitation of the mixture). The temperature was kept at 0° C. and benzyl bromide (62 mL, 516 mmol, 3 eq.) was added dropwise. The mixture was stirred overnight during which time the temperature was allowed to increase slowly to room temperature. Residual NaH was quenched carefully at 0° C. with i-PrOH. The mixture 20 was partitioned between Et₂O (800 mL) and water (800 mL). The aqueous layer was extracted 2 times with Et₂O (800 mL). The organic layers were combined, dried over MgSO₄., filtered and concentrated to afford compound 77 as yellow crystals. Recrystallization in ethanol gave compound 77 as white crystals (58 g, 73%).

### Step 3.b: Synthesis of methyl 2,3,6-tri-O-benzyl-α-D-glucopyranoside 79

In a three necked 2 L round-bottom flask, compound 77 (55 g, 119 mmol) and molecular sieves 4 Å (55 g) in dry CH₂Cl₂ (1.2 L) were stirred at room temperature for 1 h. The temperature was lowered to 0° C. and Et₃SiH (210 mL, 1.3 mol, 10.9 eq.), followed by trifluoroacetic acid (TFA) (10 mL, 130 mmol, 1.1 eq.), was added. The resultant mixture was then stirred and its temperature was allowed to increase to room temperature. The temperature was again lowered to 0° C. and TFA (10 mL, 130 mmol, 1.1 eq.) was added. The resultant mixture was stirred and the temperature was again allowed to increase to room temperature. This process (addition of TFA) was repeated three more times until optimal conversion of starting material 77 was obtained. After filtration over Celite®, the reaction mixture was diluted with CH₂Cl₂ and successively washed with water and a saturated NaHCO₃ aq. solution. The organic layer was dried over MgSO₄, filtered and concentrated. Chromatography column (ethyl acetate/heptane: 1/5 to 1/4) gave compound 79 (43.1 g, 79%).

### Step 3.b′: Synthesis of methyl 2,3-di-O-benzyl-α-D-glucopyranoside 78

In a 1 L round-bottom flask, compound 77 (15.6 g, 33.71 mmol) was dissolved in tetrahydrofuran (45 mL). Water (64 mL) and acetic acid (97 mL) were successively added and the mixture was heated overnight at 80° C. The solvent was removed by three toluene co-evaporation and the crude compound was filtered through a pad of silica (CH₂CL₂/MeOH: 90/10) to afford compound 78 (12.2 g, 97%).

### Step 3.c: Synthesis of methyl 2,3-di-O-benzyl-6-O-tert-butyldiphenylsilyl-α-D-glucopyranoside 80

In a round-bottom flask, compound 78 (12.2 g, 32.6 mmol) was dissolved in dry dichloromethane. Triethylamine (5.5 mL, 1.2 eq.), dimethylaminopyridine (398.1 mg, 0.1 eq.) and tert-butyldiphenylchlorosilane (11.9 mL, 1.4 eq.) were successively added and the resultant mixture was stirred overnight at room temperature. Well known work-up conditions were applied followed by purification on silica gel (heptane/ethyl acetate: 90/10 to 80/20) gave compound 80 (18.13 g, 91%).

¹H NMR (400 MHz, CDCl₃, ppm), δ=7.75-7.24 (m, 20H, arom.); 5.12 (d, 1H, J=11.3 Hz, CH-Ph); 4.81 and 4.78 (q_{AB}, 2H, J=10 Hz, CH₂-Ph); 4.70 (d, 1H, J=11.3 Hz, CH-Ph); 4.65 (d, 1H, J_{1,2}=3.4 Hz, H-1), 3.95-3.79 (m, 3H, H-6 and H-5); 3.71-3.57 (m, 2H, H-3 and H-4); 3.52 (dd, 1H, J_{2,3}=9.6 Hz and J_{1,2}=3.5 Hz, H-2); 3.39 (s, 3H, OMe); 1.07 (s, 9H, CH₃-tBu).

[α]_{D}=+33.8 (c=0.5, CH₂Cl₂)

### Preparation 4: Synthesis of Monosaccharides 84 and 85

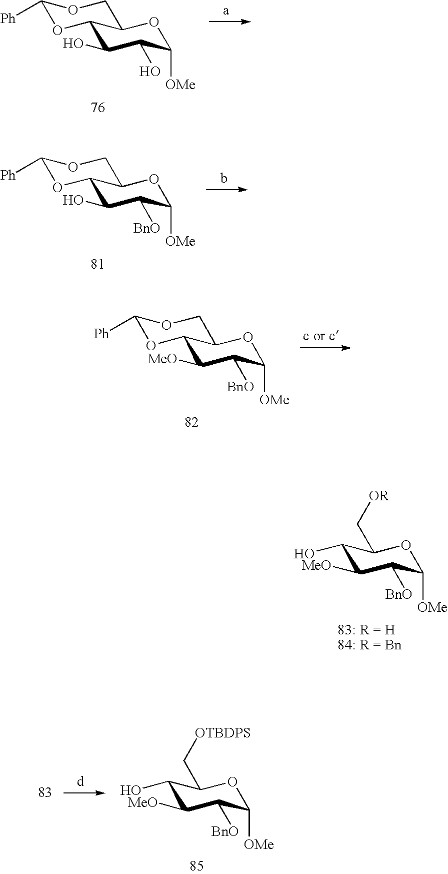

### Step 4.a: Synthesis of methyl 4,6-benzylidene-2-O-benzyl-α-D-glucopyranoside 81

In a 1 L round-bottom flask, compound 76 (35 g, 124 mmol) was dissolved in dry dimethylformamide (350 mL) and cooled to 0° C. Sodium hydride (60% in oil, 5.95 g, 147 mmol, 1.2 eq.) was added by portion and the suspension was stirred 1 h at 0° C. Then, benzyl bromide (17.7 mL, 149 mmol, 1.2 eq.) was added slowly. After stirring for 2 h at room temperature, residual NaH was quenched by addition of methanol (20 mL). The mixture was diluted in dichloromethane (1.5 mL) and washed successively with water (700 mL), a NaHCO₃ saturated aqueous solution (700 mL) and water (700 mL). The organic layer was dried over MgSO₄, filtered and concentrated under vacuum. Purification of the crude compound by silica chromatography (heptane/ethyl acetate: 85/15 to 50/50) gave compound 81 (28.6 g, 62%).

¹H NMR (400 MHz, CDCl₃, ppm), δ=7.55-7.29 (m, 10H, arom.); 5.53 (s, 1H, CH-Ph); 4.80 and 4.72 (q_{AB}, 2H, J=11.9 Hz, CH₂-Ph); 4.63 (d, 1H, J_{1,2}=3.4 Hz, H-1), 4.26 (dd, 1H, J_{6a,6b}=9.4 Hz and J_{5,6a}=5.2 Hz, H-6a); 4.17 (dd, 1H, J_{6a,6b}=9.4 Hz and J_{5,6b}=2.0 Hz, H-6b); 3.83 (m, 1H, H-5); 3.72 (t, 1H, J_{3,4}=J_{4,5}=9.7 Hz, H-4); 3.51 (t, 1H, J_{3,4}=J_{2,3}=9.7 Hz, H-3); 3.49 (dd, 1H, J_{2,3}=9.7 Hz and J_{1,2}=3.3 Hz, H-2); 3.39 (s, 3H, OMe).

### Step 4.b: Synthesis of methyl 4,6-benzylidene-2-O-benzyl-3-O-methyl-α-D-glucopyranoside 82

In a 500 mL round-bottom flask, under an atmosphere of Ar, compound 81 (28.6 g, 76.8 mmol) was dissolved in dry DMF (180 mL). The solution was cooled at 0° C. and then NaH (60% in oil, 3.84 g, 96 mmol, 1.25 eq.) was added slowly. Methyl bromide (12.77 mL, 115.2 mmol, 1.5 eq.) was added dropwise and the resultant mixture was stirred overnight and the reaction temperature was allowed to increase to room temperature. Residual NaH was then quenched carefully at 0° C. with methanol (30 mL) followed by addition of a Na₂S₂O₃ saturated aqueous solution (100 mL) to quench residual I₂. The mixture was diluted with ethyl acetate (800 mL) and the organic layer was successively washed with a NaCl saturated aqueous solution (3×700 mL) and water (1×700 mL). The organic layer was dried over MgSO₄, filtered and concentrated to afford crude compound 82 (31.0 g) which was carried on to the next synthetic step without any further purification.

¹H NMR (400 MHz, CDCl₃, ppm), δ=7.57-7.27 (m, 10H, arom.); 5.54 (s, 1H, CH-Ph); 4.89 and 4.70 (q_{AB}, 2H, J=12.1 Hz, CH₂-Ph); 4.60 (d, 1H, J_{1,2}=3.7 Hz, H-1), 4.27 (dd, 1H, J_{6a,6b}=9.9 Hz and J_{5,6a}=4.6 Hz, H-6a); 3.88-3.70 (m, 3H, H-4, H-5, H-6b); 3.52 (t, 1H, J_{3,4}=J_{2,3}=9.3 Hz, H-3); 3.49 (dd, 1H, J_{2,3}=9.7 Hz and J_{1,2}=3.7 Hz, H-2); 3.41 (s, 3H, OMe).

[α]_{D}=+47.6 (c=0.5, CH₂Cl₂)

### Step 4.c: Synthesis of methyl 2-O-benzyl-3-O-methyl-α-D-glucopyranoside 83

This compound was produced using the same procedure used in the preparation of monosaccharide 78 described above. Compound 83 was used directly in the next step without any further purification.

### Step 4.c′: Synthesis of methyl 2,6-di-O-benzyl-3-O-methyl-α-D-glucopyranoside 84

This compound was produced using the same procedure used in the preparation of monosaccharide 79 described above.

### Step 4.d: Synthesis of methyl 2-O-benzyl-3-O-methyl-6-O-tert-butyldimethylsilyl-α-D-glucopyranoside 85

This compound was produced using the same procedure used in the preparation of monosaccharide 80 described above.

¹H NMR (400 MHz, CDCl₃, ppm), δ=7.74-7.26 (m, 15H, arom.); 4.79 and 4.67 (q_{AB}, 2H, J=12.2 Hz, CH₂-Ph); 4.60 (d, 1H, J_{1,2}=3.4 Hz, H-1), 3.87 (m, 2H, H-6); 3.71 (s, 3H, OMe); 3.67 (m, 1H, H-5); 3.56 (m, 2H, H-3 et H-4); 3.42 (m, 1H, H-2); 3.36 (s, 3H, OMe); 1.07 (s, 9H, CH₃-tBu).

[α]_{D}=+47 (c=0.5, CH₂Cl₂)

### Preparation of Disaccharides

### Preparation 5: Synthesis of Disaccharide 90

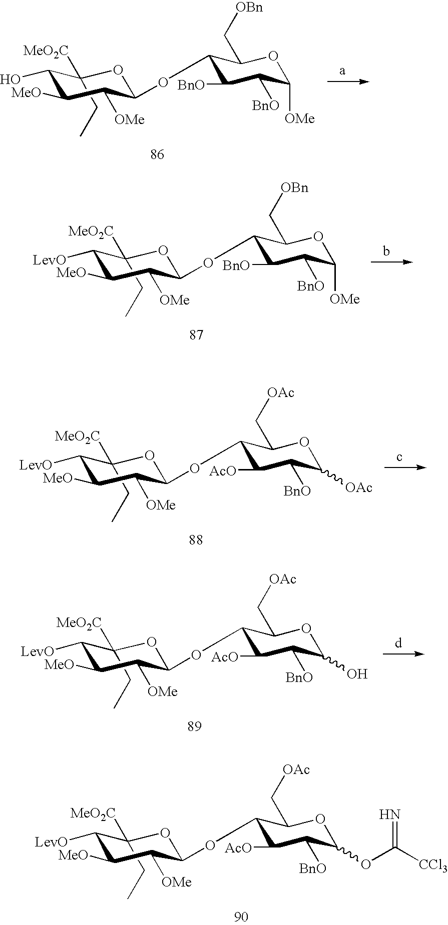

### Step 5.a: Synthesis of methyl O-(methyl-4-O-levulinyl-2,3-di-O-methyl-5-C-ethyl-β-D-glucopyranosyluronate)-(1→4)-O-2,3,6-tri-O-benzyl-α-D-glucopyranoside 87

The synthesis of compound 86 is described in Das, S. K. et al. Chem. Eur. J. 2001, 7, 4821-4833.

Compound 86 (150 mg, 0.21 mmol) was placed in a dry round-bottom flask and dissolved in anhydrous dichloromethane (1.6 mL). Levulinic acid (49 mg, 2 eq.) followed by EDAC (81 mg, 2 eq.) was added to the solution, which was stirred at room temperature under nitrogen. After 5 min, DMAP (5.2 mg, 0.2 eq.) was added and the reaction mixture was stirred overnight at room temperature. The organic layer was diluted with dichloromethane (40 mL), washed with saturated aqueous solutions of NH₄Cl and NaHCO₃, dried over MgSO₄, filtered and concentrated under reduced pressure. Purification was performed using silica gel column chromatography (Heptane/AcOEt: 1:1) to give compound 87 (101 mg, 59%).

¹H NMR (400 MHz, CDCl₃, ppm), δ=4.62 (d, 1H, J=8.0 Hz, H-1), 4.61 (s, 1H, H-1′), 2.75-2.62 (m, 4H, CH₂-Lev), 2.18 (s, 3H, CH₃-Lev).

### Step 5.b: Synthesis of acetyl O-(methyl-4-O-levulinyl-2,3-di-O-methyl-5-C-ethyl-β-D-glucopyranosyluronate)-(1→4)-O-1,3,6-tri-O-acetyl-2-O-benzyl-α,β-D-glucopyranoside 88

Compound 87 (101 mg, 0.125 mmol) was placed in a dry round-bottom flask and suspended in acetic anhydride (8 mL) before being cooled in an ice-bath. After 15 min, H₂SO₄ (100 μL at 5% in AcOH) was added. The reaction was stirred at room temperature for 3 h and concentrated to ⅔ of its initial volume. EtOAc (50 mL) was added and the organic layer was washed with a saturated aqueous solution of NaHCO₃, dried on MgSO₄ and concentrated under reduced pressure. Purification by silica gel column chromatography (Toluene/AcOEt: 7:3) gave compound 88 (61 mg, 66%), ratio α/β=3:1.

¹H NMR (400 MHz, CDCl₃, ppm), δ=6.32 (d, 0.75H, J=3.5 Hz, H-1α), 5.65 (d, 0.25H, J=7.7 Hz, H-1β), 5.46 (t, 0.25H, J=9.7 Hz, H-3β), 5.28 (t, 0.75H, J=8.8 Hz, H-3α), 4.45 (d, 1H, J=7.7 Hz, H-1′), 2.16, 2.10, 2.09 (3s, 9H, CH₃).

### Step 5.c: Synthesis of methyl-O-(methyl-4-O-levulinyl-2,3-di-O-methyl-5-C-ethyl-β-D-glucopyranosyluronate)-(1→4)-O-3,6-di-O-acetyl-2-O-benzyl-α,β-D-glucopyranoside 89

In a dry round-bottom flask was introduced compound 88 (350 mg, 0.405 mmol) in a mixture of THF/MeOH (7/3, 500 μL) and cooled to 0° C. After stirring for 15 min, the solution was bubbled with a gentle flow of ammonia for 35 min (TLC showed disappearance of the starting material). The reaction mixture was then purged with nitrogen for 20 min and concentrated to dryness under reduced pressure. The crude mixture was purified using silica gel column chromatography (toluene/AcOEt: 5/7) to give compound 89 (229 mg, 81%).

### Step 5.d: Synthesis of methyl-O-(methyl-4-O-levulinyl-2,3-di-O-methyl-5-C-ethyl-β-D-glucopyranosyluronate)-(1→4)-O-2-O-benzyl-3,6-di-O-acetyl-α,β-D-glucopyranose trichloroacetimidate 90

In a dry round-bottom flask, compound 89 (229 mg, 0.328 mmol) was dissolved in dichloromethane (1 mL), followed by addition of Cs₂CO₃ (149 mg, 8 eq.) and CCl₃CN (265 μL, 8 eq.). After stirring at room temperature for 20 min, the starting material was consumed but the solution was kept under stirring for another 40 min until the ratio α/β stayed stable. The reaction mixture was diluted in dichloromethane, filtered through a pad of Celite®, washed and the filtrate was concentrated to ⅔ of its initial volume. Water was added and the aqueous layer was extracted with dichloromethane, dried over MgSO₄ and concentrated under reduced pressure to give compound 90 as a light yellow solid (205 mg, 74%) ratio α/β=25:75.

¹H NMR (400 MHz, CDCl₃, ppm), δ=8.55 (s, 0.75H, NHβ), 8.7 (s, 0.25H, NHα), 6.6 (d, 0.75H, J=3.30 Hz, H-1β), 5.85 (d, 0.25H, J=7.80 Hz, H-1α).

### Preparation 6: Synthesis of Disaccharide 94

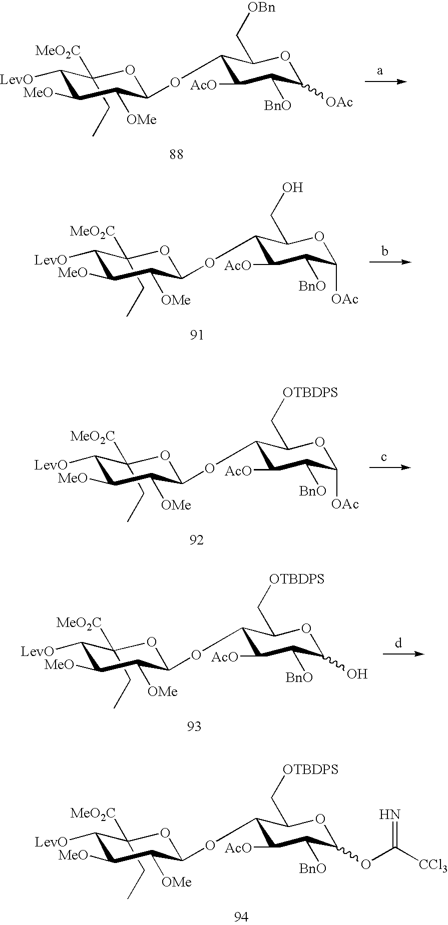

### Step 6.a: Synthesis of methyl O-(methyl-4-O-levulinyl-2,3-di-O-methyl-5-C-ethyl-β-D-glucopyranosyluronate)-(1→4)-O-1,3-di-O-acetyl-2-O-benzyl-α-D-glucopyranoside 91

In a 250 mL round bottom flask, compound 88 (5 g, 6.75 mmol) was dissolved in a THF/MeOH (1/1) mixture (72 mL) and [tert-BuSnOH(Cl)]₂ (190.7 mg, 0.34 mmol, 0.05 eq.) was subsequently added. After the mixture was heated overnight at 30° C., it was concentrated to dryness under vacuum. The resultant residue was purified by silica gel chromatography (toluene/Ethyl acetate: 5/5) to give compound 91 as an amorphous white powder (3.26 g, 69%).

¹H NMR (400 MHz, CDCl₃, ppm), δ=7.38-7.14 (m, 5H, arom.), 6.29 (d, 1H, J_{1,2}=3.4 Hz, H-1), 5.47 (t, 1H, J_{2,3}=J_{3,4}=9.5 Hz, H-3), 5.14 (d, 1H, J_{3′,4′}=8.5 Hz, H-4′); 4.64 and 4.55 (q_{AB}, 2H, J=12 Hz, CH₂Ph); 4.61 (d, 1H, J_{1′,2′}=7.4 Hz, H-1′); 4.03-3.77 (m, 4H); 3.68 (s, 3H, CO₂Me); 3.60 (dd, 1H, J₁=9.5 hz, J₂=3.5 Hz); 3.52 and 3.13 (s, 6H, OMe); 3.36 (t, 1H, J=8.2 Hz); 3.13 (t, 1H, J=8.0 Hz); 2.61 (m, 2H, CH₂—CH₂COCH₃); 2.61 (m, 2H, CH₂—CH₂COCH₃); 2.20 (s, 3H, CH₂—CH₂COCH₃); 2.17 and 2.11 (2s, 6H, CH₃Ac); 1.97 (m, 1H, CH—CH₃); 1.11 (m, 1H, CH—CH₃); 0.92 (t, 3H, J=7.8 Hz, CH—CH₃).

### Step 6.b: Synthesis of methyl O-(methyl-4-O-levulinyl-2,3-di-O-methyl-5-C-ethyl-β-D-glucopyranosyluronate)-(1→4)-O-1,3-di-O-acetyl-2-O-benzyl-6-O-tert-butyldiphenylsilyl-α-D-glucopyranoside 92

In a 100 mL round bottom flask, compound 91 (2.96 g, 4.24 mmol) was dissolved in dichloromethane (15 mL). Tert-butyldiphenylchlorosilane (5.5 mL, 21.2 mmol, 5 eq.), triethylamine (3 mL, 21.2 mmol, 5 eq.) and 4-dimethylaminopyridine (258.4 mg, 2.1 mmol, 0.5 eq.) were successively added and the reaction mixture was stirred overnight at room temperature. Dilution in dichloromethane was followed by well known work-up procedures to give a crude residue (8.9 g). Purification by silica chromatography (toluene/ethyl acetate: 8/2 to 5/5) gave compound 92 as a white amorphous solid (3.53 g, 89%).

¹H NMR (400 MHz, CDCl₃, ppm), δ=7.80-7.67 (m, 4H, arom.); 7.48-7.13 (m, 11H, arom.), 6.40 (d, 1H, J_{1,2}=3.6 Hz, H-1), 5.48 (t, 1H, J_{2,3}=J_{3,4}=9.7 Hz, H-3), 5.12 (d, 1H, J_{3′,4′}=9.2 Hz, H-4′); 4.70 and 4.55 (q_{B}, 2H, J=12 Hz, CH₂Ph); 4.64 (d, 1H, J_{1′,2′}=8.1 Hz, H-1′); 4.46 (dd, 1H, J₁=11.5 Hz and J₂=1.8 Hz); 4.02 (t, 1H, J=10 Hz); 3.86 (m, 2H); 3.65 (s, 3H, CO₂Me); 3.60 (m, 1H); 3.55 and 3.47 (2s, 6H, OMe); 3.31 (t, 1H, J=9.2 Hz); 3.13 (t, 1H, J=8.4 Hz); 2.73 (m, 2H, CH₂—CH₂COCH₃); 2.54 (m, 2H, CH₂—CH₂COCH₃); 2.18 (s, 3H, CH₂—CH₂COCH₃); 2.13 and 2.05 (2s, 6H, CH₃Ac); 2.03 (m, 1H, CH—CH₃); 1.65 (m, 1H, CH—CH₃); 1.07 (s, 9H, CH₃-tBu); 0.87 (t, 3H, J=7.6 Hz, CH—CH₃).

### Step 6.c: Synthesis of methyl O-(methyl-4-O-levulinyl-2,3-di-O-methyl-5-C-ethyl-β-D-glucopyranosyluronate)-(1→4)-O-3-O-acetyl-2-O-benzyl-6-O-tert-butyldiphenylsilyl-α,β-D-glucopyranoside 9

Compound 93 was prepared according to general Method G. Compound 93 (3.36 g) was used in the next synthetic step without any further purification.

ESI-MS, positive mode: 918.35 [M+Na⁺]; 933.38 [M+K⁺].

### Step 6.d: Synthesis of methyl O-(methyl-4-O-levulinyl-2,3-di-O-methyl-5-C-ethyl-β-D-glucopyranosyluronate)-(1→4)-O-3-O-acetyl-2-O-benzyl-6-O-tert-butyldiphenylsilyl-α,β-D-glucopyranose trichloroacetimidate 94

Compound 94 was prepared according to general Method H. Purification by silica gel chromatography (toluene/ethyl acetate: 8/2+1% of triethylamine) gave compound 94 as a white amorphous powder (α/β: 6/4, 3.59 g, 92%).

### Isomer α:

¹H NMR (400 MHz, CDCl₃, ppm), δ=8.57 (s, 1H, NH); 7.81-7.69 (m, 4H, arom.); 7.50-7.12 (m, 11H, arom.), 6.59 (d, 1H, J_{1,2}=3.6 Hz, H-1), 5.62 (t, 1H, J_{2,3}=J_{3,4}=9.7 Hz, H-3), 5.13 (d, 1H, J_{3′,4′}=8.9 Hz, H-4′); 4.74 and 4.62 (q_{AB}, 2H, J=12.5 Hz, CH₂Ph); 4.62 (d, 1H, J_{1′,2′}=8.5 Hz, H-1′); 4.34 (d, 1H, J=10.7 Hz); 4.09-3.89 (m, 3H); 3.71 (dd, 1H, J₁=10.4 Hz and J₂=3.2 Hz); 3.66 (s, 3H, CO₂Me); 3.55 and 3.44 (2s, 6H, OMe); 3.30 (t, 1H, J=8.9 Hz); 3.16 (t, 1H, J=8.6 Hz); 2.73 (m, 2H, CH₂—CH₂COCH₃); 2.55 (m, 2H, CH₂—CH₂COCH₃); 2.19 (s, 3H, CH₂—CH₂COCH₃); 2.19 (s, 3H, CH₃Ac); 2.04 (m, 1H, CH—CH₃); 1.64 (m, 1H, CH—CH₃); 1.08 (s, 9H, CH₃-tBu); 0.87 (t, 3H, J=7.8 Hz, CH—CH₃).

### Isomer β

¹H NMR (400 MHz, CDCl₃, ppm), δ=8.71 (s, 1H, NH); 7.81-7.69 (m, 4H, arom.); 7.49-7.14 (m, 11H, arom.), 5.89 (d, 1H, J_{1,2}=7.8 Hz, H-1), 5.35 (t, 1H, J_{2,3}=J_{3,4}=9.5 Hz, H-3), 5.13 (d, 1H, J_{3′,4′}=9 Hz, H-4′); 3.66 (s, 3H, CO₂Me); 3.57 and 3.47 (2s, 6H, OMe); 2.20 (s, 3H, CH₂—CH₂COCH₃); 2.01 (s, 3H, CH₃Ac); 1.07 (s, 9H, CH₃-tBu); 0.87 (t, 3H, J=7.8 Hz, CH—CH₃).

### Preparation of Tetrasaccharides

### Preparation 7: Synthesis of Tetrasaccharides 97, 98 and 100

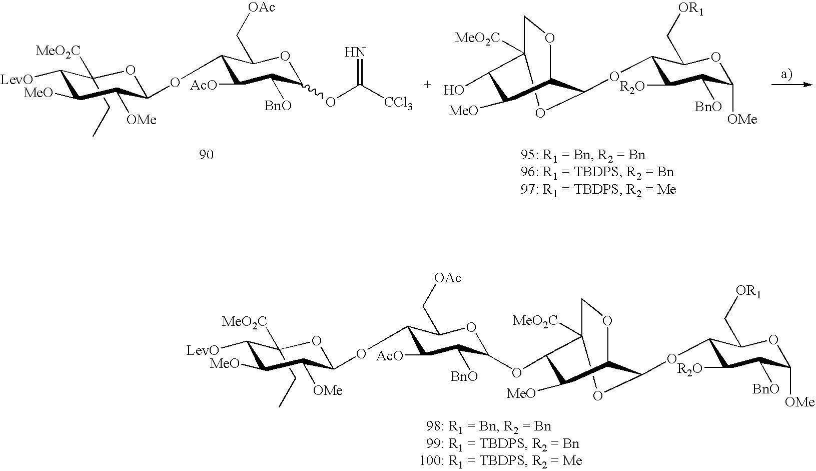

The synthesis of compounds 95, 96 and 97 is described in Das, S. K. and al. Chem. Eur. J. 2001, 7, 4821-4833.

### Synthesis of methyl O-(methyl-4-O-levulinyl-2,3-di-O-methyl-5-C-ethyl-β-D-glucopyranosyluronate)-(1→4)-O-(3,6-di-O-acetyl-2-O-benzyl-α-D-glucopyranosyl)-(1→4)-O-(methyl-2,6-anhydro-3-O-methyl-β-D-mannopyranosyluronate)-(1→4)-O-2,3,6-tri-O-benzyl-α-D-glucopyranoside 98

Coupling of disaccharide 90 (246 mg, 1.1 eq.) with disaccharide 95 (181 mg, 0.266 mmol) was performed as described in Method I, which gave, after purification, compound 98 (202 mg, 56%).

¹H NMR (400 MHz, CDCl₃, ppm), δ=5.13 (s, 1H, H-1 ManUA^{II}), 4.93 (s, 1H, H-1 Glc^{III}), 4.54 (d, 1H, J=8.7 Hz, H-1 GlcUA^{IV}), 4.49 (1H, d, J=3.4 Hz, H-1 Glc^{I}).

### Synthesis of methyl O-(methyl-4-O-levulinyl-2,3-di-O-methyl-5-C-ethyl-β-D-glucopyranosyluronate)-(1→4)-O-(3,6-di-O-acetyl-2-O-benzyl-α-D-glucopyranosyl)-(1→4)-O-(methyl-2,6-anhydro-3-O-methyl-β-D-mannopyranosyluronate)-(1→4)-O-2,3-di-O-benzyl-6-O-tert-butyldiphenylsilyl-α-D-glucopyranoside 99

Coupling of disaccharide 90 (250 mg, 0.297 mmol, 1 eq.) with disaccharide 96 (368.7 mg, 0.445 mmol, 1.5 eq.) was performed as described in Method I, which gave, after purification, compound 99 (354.6 mg, 68%).

¹H NMR (400 MHz, CDCl₃, ppm), δ=7.74-7.12 (m, 25H, arom.); 5.38 (t, 1H, J_{2,3}=J_{3,4}=9.7 Hz, H-3 Glc^{III}); 5.23 (sl, 1H, H-1 ManUA^{II}), 5.10 (d, 1H, J_{1,2}=8.9 Hz, H-4 GlcUA^{IV}); 5.04-4.88 (m, 3H, CH₂-Ph, H-1 Glc^{III}); 4.8 and 4.69 (q_{AB}, 2H, J=12 Hz, CH₂-Ph); 4.64-4.50 (m, 4H, CH₂-Ph, H-1 GlcUA^{IV} and H-1 Glc^{I}); 3.67 (s, 6H, CO₂Me); 3.49, 3.47 and 3.36 (3s, 9H, OMe); 2.76 (m, 2H, CH₂—CH₂—COCH₃); 2.60 (m, 2H, CH₂—CH₂—COCH₃); 2.20 (s, 3H, CH₂—CH₂—COCH₃); 2.11 and 2.09 (s, 6H, CH₃—OAc); 1.73 (m, 1H, CH—CH₃); 1.03 (s, 9H, CH₃-tBu); 0.93 (t, 3H, J=8.1 Hz, CH₃—CH).

MALDI-MS, m/z: 1554.08 [M+Na]⁺, 1547.98 [M+K]⁺

### Synthesis of methyl O-(methyl-4-O-levulinyl-2,3-di-O-methyl-5-C-ethyl-β-D-glucopyranosyluronate)-(1→4)-O-(3,6-di-O-acetyl-2-O-benzyl-α-D-glucopyranosyl)-(1→4)-O-(methyl-2,6-anhydro-3-O-methyl-β-D-mannopyranosyluronate)-(1→4)-O-2-O-benzyl-3-O-methyl-6-O-tert-butyldiphenylsilyl-α-D-glucopyranoside 100

Coupling of disaccharide 90 (2 g, 2.37 mmol, 1 eq.) with disaccharide 97 (2.3 g, 3.08 mmol, 1.3 eq.) was performed as described in Method I, which gave, after purification, compound 100 (2.55 g, 75%).

¹H NMR (400 MHz, CDCl₃, ppm), δ=7.73-7.11 (m, 20H, arom.); 5.35 (t, 1H, J_{2,3}=J_{3,4}=9.6 Hz, H-3 Glc^{III}); 5.11 (d, 1H, J_{1,2}=7.6 Hz, H-4 GlcUA^{rv}); 5.10 (sl, 1H, H-1 ManUA^{II}); 5.04 (d, 1H, J_{1,2}=3.4 Hz, H-1 Glen; 4.85 and 4.71 (q_{AB}, 2H, J=12.4 Hz, CH₂-Ph); 4.68-4.51 (m, 4H, CH₂-Ph, H-1 GlcUA^{Iv} and H-1 Glc^{I}); 3.81 and 3.70 (2s, 6H, CO₂Me); 3.67, 3.54, 3.35 and 3.10 (5s, 15H, OMe); 2.75 (m, 2H, CH₂—CH₂—COCH₃); 2.60 (m, 2H, CH₂—CH₂—COCH₃); 2.20 (s, 3H, CH₂—CH₂—COCH₃); 2.10 (s, 6H, CH₃—OAc); 1.73 (m, 1H, CH—CH₃); 1.03 (s, 9H, CH₃-tBu); 0.93 (t, 3H, J=8.1 Hz, CH₃—CH).

### Preparation 8: Synthesis of Tetrasaccharides 103 and 104

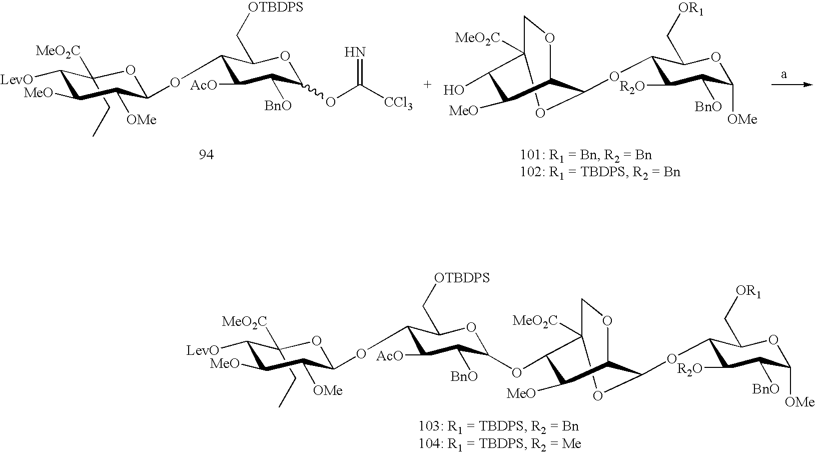

### Synthesis of methyl O-(methyl-4-O-levulinyl-2,3-di-O-methyl-5-C-ethyl-β-D-glucopyranosyluronate)-(1→4)-O-(3-O-acetyl-2-O-benzyl-6-O-tert-butyldiphenylsilyl-α-D-glucopyranosyl)-(1→4)—O-(methyl-2,6-anhydro-3-O-methyl-β-D-mannopyranosyluronate)-(1→4)-O-2,3-di-O-benzyl-6-O-tert-butyldiphenylsilyl-α-D-glucopyranoside 103

Coupling of disaccharide 94 (0.5 g, 0.48 mmol, 1 eq.) with disaccharide 101 (0.598 g, 0.72 mmol, 1.5 eq.) was performed as described in Method I, which gave, after purification, compound 103 (1.15 g, 70%).

¹H NMR (400 MHz, CDCl₃, ppm), δ=7.82-7.15 (m, 35H, arom.); 5.42 (t, 1H, J_{2,3}=J_{3,4}=9.7 Hz, H-3 Glc^{III}); 5.19 (sl, 1H, H-1 ManUA^{II}), 5.11 (d, 1H, J_{1,2}=9.2 Hz, H-4 GlcUA^{IV}); 4.99 and 4.91 (q_{AB}, 2H, J=10.1 Hz, CH₂-Ph); 4.99 (sl, 1H, H-1 Glc^{III}); 4.8 and 4.69 (q_{AB}, 2H, J=12 Hz, CH₂-Ph); 4.66-4.50 (m, 4H, CH₂-Ph, H-1 GlcUA^{IV} and H-1 Glc^{I}); 3.67 (s, 3H, CO₂Me); 3.55 (s, 3H, CO₂Me); 3.47, 3.45, 3.34 and 3.02 (4s, 12H, OMe); 2.73 (m, 2H, CH₂—CH₂—COCH₃); 2.55 (m, 2H, CH₂—CH₂—COCH₃); 2.18 (s, 3H, CH₂—CH₂—COCH₃); 2.04 (s, 3H, CH₃—OAc); 1.60 (m, 1H, CH—CH₃); 1.08 and 1.03 (s, 18H, CH₃-tBu); 0.85 (t, 3H, J=8.1 Hz, CH₃—CH).

### Synthesis of methyl O-(methyl-4-O-levulinyl-2,3-di-O-methyl-5-C-ethyl-β-D-glucopyranosyluronate)-(1→4)-O-(3-O-acetyl-2-O-benzyl-6-O-tert-butyldiphenylsilyl-α-D-glucopyranosyl)-(1→4)-O-(methyl-2,6-anhydro-3-O-methyl-β-D-mannopyranosyluronate)-(1→4)-O-2-O-benzyl-3-O-methyl-6-O-tert-butyldiphenylsilyl-α-D-glucopyranoside 104

Coupling of disaccharide 94 (1.13 g, 1.09 mmol, 1 eq.) with disaccharide 102 (1.23 g, 1.63 mmol, 1.3 eq.) was performed as described in Method I, which gave, after 25 purification, compound 104 (2.55 g, 93%).

¹H NMR (400 MHz, CDCl₃, ppm), δ=7.81-7.25 (m, 25H, arom.); 5.34 (t, 1H, J_{2,3}=J_{3,4}=10 Hz, H-3 Glc^{III}); 5.12 (d, 1H, J_{1,2}=9.8 Hz, H-4 GlcUA^{IV}); 5.06 (sl, 1H, H-1 ManUA^{II}), 5.03 (d, 1H, J_{1,2}=3.3 Hz, H-1 Glc^{III}); 4.88-4.53 (6H, 2*CH₂-Ph, H-1 GlcUA^{IV} and H-1 Glc^{I}); 3.66 (2s, 6H, CO₂Me); 3.59, 3.56, 3.50, 3.34 and 3.12 (5s, 15H, OMe); 2.73 (m, 2H, CH₂—CH₂—COCH₃); 2.55 (m, 2H, CH₂—CH₂—COCH₃); 2.19 (s, 3H, CH₂—CH₂—COCH₃); 2.01 (s, 3H, CH₃—OAc); 1.62 (m, 1H, CH—CH₃); 1.08 and 1.01 (s, 18H, CH₃-tBu); 0.85 (t, 3H, J=7.7 Hz, CH₃—CH).

### Preparation 9: Synthesis of Tetrasaccharide 107

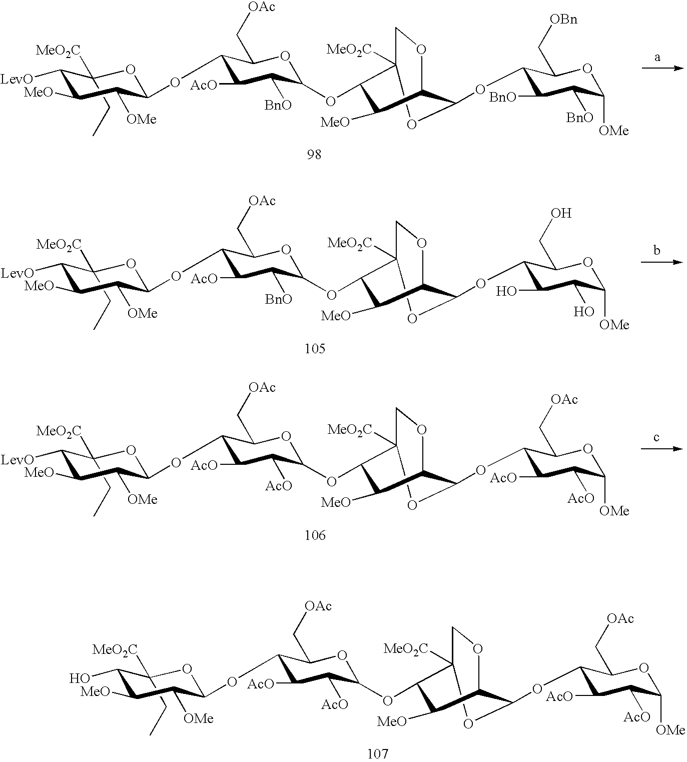

### Step 9.a: Synthesis of methyl O-(methyl-4-O-levulinyl-2,3-di-O-methyl-5-C-ethyl-β-D-glucopyranosyluronate)-(1→4)-O-(3,6-di-O-acetyl-α-D-glucopyranosyl)-(1→4)-O-(methyl-2,6-anhydro-3-O-methyl-β-D-mannopyranosyluronate)-(1→4)-O-α-D-glucopyranoside 105

In a dry round-bottom flask, compound 98 (256 mg, 0.147 mmol) was dissolved in a 1/1 mixture of anhydrous THF and absolute EtOH (total volume, 26 mL) and Pd(OH)₂ (256 mg, 1 weight eq.) was added. After stirring for 10 min at 0° C., the reaction mixture was purged three times with hydrogen and left overnight at room temperature under an atmosphere of hydrogen. The reaction mixture was filtered and concentrated under reduced pressure to give compound 105 (310 mg) which was directly used in the next step.

### Step 9.b: Synthesis of crude methyl O-(methyl-4-O-levulinyl-2,3-di-O-methyl-5-C-ethyl-β-D-glucopyranosyluronate)-(1→4)-O-(2,3,6-tri-O-acetyl-α-D-glucopyranosyl)-(1→4)-O-(methyl-2,6-anhydro-3-O-methyl-β-D-mannopyranosyluronate)-(1→4)-O-2,3,6-tri-O-acetyl-α-D-glucopyranoside 106

Compound 105 (456 mg, 0.456 mmol) was placed in a dry round-bottom flask in anhydrous dichloromethane (2.5 mL) followed by addition at room temperature of DMAP (110 mg, 2 eq.), triethylamine (1.4 mL, 22 eq.) and acetic anhydride (861 μL, 20 eq.). After stirring for 2 h, the reaction mixture was diluted with dichloromethane (500 mL). The organic layer was successively washed with a 5% H₂SO₄ solution, water and a saturated aqueous solution of NaHCO₃, dried over MgSO₄ and concentrated under reduced pressure to give crude compound 106 (544 mg), which was used in the next step without any further purification.

¹H NMR (400 MHz, CDCl₃, ppm), δ=5.33 (t, 1H, J=10.1 Hz, H-3 Glc^{I}), 5.22 (d, 1H, J=3.6 Hz, H-1 Glc^{III}), 4.86-4.87 (bs, 2H, H-1 ManUA^{II}, H-1 Glc^{I}), 4.73 (dd, 1H, J=3.6, 10.1 Hz, H-2 Glc^{III}), 4.81 (dd, 1H, J=3.0, 10.1 Hz, H-2 Glc^{I}), 4.35 (d, 1H, J=7.8 Hz, H-1 GlcUA^{IV}).

### Step 9.c: Synthesis of methyl O-(methyl-2,3-di-O-methyl-5-C-ethyl-β-D-glucopyranosyluronate)-(1→4)-O-(2,3,6-tri-O-acetyl-α-D-glucopyranosyl)-(1→4)-O-(methyl-2,6-anhydro-3-O-methyl-β-D-mannopyranosyluronate)-(1→4)-O-2,3,6-tri-O-acetyl-α-D-glucopyranoside 107

In a dry round-bottom flask compound 106 (221 mg, 0.189 mmol) was dissolved in a mixture of both anhydrous methanol and dichloromethane (2/1, 3.6 mL). Hydrazine acetate (35 mg, 2 eq.) was added to the reaction mixture at room temperature. The reaction was stirred for 4 h and diluted in dichloromethane (30 mL). The organic layer was successively washed with a 5% H₂SO₄ solution and a saturated aqueous solution of NaHCO₃, dried over MgSO₄, filtered and concentrated under reduced pressure. Silica gel column chromatography (Toluene/Acetone, 7/3) gave compound 107 (122 mg, 55% over three steps). ¹H NMR (400 MHz, CDCl₃, ppm), δ=5.33 (t, 1H, J=10.1 Hz, H-3 Glc^{I}), 5.22 (d, 1H, J=3.6 Hz, H-1 Glc^{III}), 4.86-4.87 (bs, 2H, H-1 ManUA^{II}, H-1 Glc^{I}), 4.81 (dd, 1H, J=3.0, 10.1 Hz, H-2 Glc^{I}), 4.73 (dd, 1H, J=3.6, 10.1 Hz, H-2 Glc^{III}), 4.35 (d, 1H, J=7.8 Hz, H-1 GlcUA^{IV}), 2.85 (bs, 1H, OH GlcUA^{IV}).

### Preparation 10: Synthesis of Tetrasaccharides 112 and 113

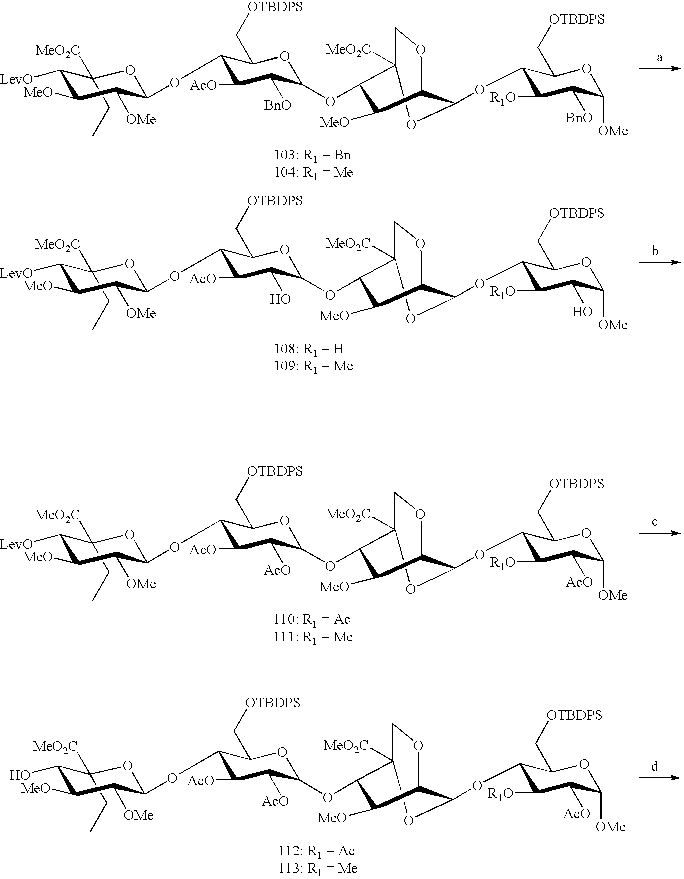

Tetrasaccharides 112 and 113 were prepared following the same procedure that was used for the preparation of tetrasaccharide 107.

### Methyl O-(methyl-2,3-di-O-methyl-5-C-ethyl-β-D-glucopyranosyluronate)-(1→4)-O-(2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-O-2,3-di-O-acetyl-6-O-tert-butyldiphenylsilyl-α-D-glucopyranoside 112

¹H NMR (400 MHz, CDCl₃, ppm), δ=7.81-7.32 (m, 20H, arom.); 5.57 (t, 1H, J_{2,3}=J_{3,4}=9.0 Hz, H-3 Glc^{I}); 5.36 (t, 1H, J_{2,3}=J_{3,4}=9.5 Hz, H-3 Glc^{III}); 5.32 (sl, 1H, H-1 Glc^{III}); 4.95 (sl, 1H, H-1 ManUA^{II}); 4.92 (d, 1H, J_{1,2}=3.8 Hz, H-1 Glc^{I}); 4.84 (dd, 1H, J_{1,2}=3.5 Hz and J_{2,3}=9.0 Hz, H-2 Glc^{I}); 4.77 (dd, 1H, J_{1,2}=3.5 Hz and J_{2,3}=9.5 Hz, H-2 Glc^{III}); 4.60 (d, 1H, J_{1,2}=7.7 Hz, H-1 GlcUA^{IV}); 3.76 and 3.67 (2s, 6H, CO₂Me); 3.58, 3.47, 3.35 and 3.29 (4s, 12H, OMe); 3.12 (sl, 1H, OH-GlcUA^{IV}); 2.10, 2.09, 2.04 and 2.03 (s, 12H, CH₃—OAc); 1.62 (m, 1H, CH—CH₃); 1.086 and 1.06 (s, 18H, CH₃-tBu); 0.86 (t, 3H, J=7.3 Hz, CH₃—CH).

MALDI-MS, m/z: 1486.71 [M+Na]⁺, 1501.71 [M+K]⁺

### Methyl O-(methyl-2,3-di-O-methyl-5-C-ethyl-β-D-glucopyranosyluronate)-(1→4)-O-(2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-O-2-O-acetyl-3-O-methyl-6-O-tert-butyldiphenylsilyl-α-D-glucopyranoside 113

¹H NMR (400 MHz, CDCl₃, ppm), δ=7.81-7.13 (m, 20H, arom.); 5.38 (t, 1H, J_{2,3}=J_{3,4}=9.5 Hz, H-3 Glc^{III}); 5.35 (d, 1H, J_{1,2}=3.7 Hz, H-1 Glc^{III}); 5.15 (sl, 1H, H-1 ManUA^{II}), 4.90 (d, 1H, J_{1,2}=3.6 Hz, H-1 Glc^{I}); 4.80 (dd, 1H, J_{1,2}=3.6 Hz and J_{2,3}=9.5 Hz, H-2 Glc^{I}); 4.76 (dd, 1H, J_{1,2}=3.9 Hz and J_{2,3}=9.5 Hz, H-2 Glc^{III}); 4.60 (1H, H-1 GlcUA^{IV}); 3.76 and 3.67 (2s, 6H, CO₂Me); 3.64, 3.59, 3.48, 3.33 and 3.28 (5s, 15H, OMe); 2.82 (sl, 1H, OH-GlcUA^{IV}); 2.17, 2.05 and 2.02 (s, 9H, CH₃—OAc); 1.62 (m, 1H, CH—CH₃); 1.08 and 1.06 (s, 18H, CH₃-tBu); 0.86 (t, 3H, J=7.7 Hz, CH₃—CH).

### Preparation 11: Synthesis of Tetrasaccharides 120 and 121

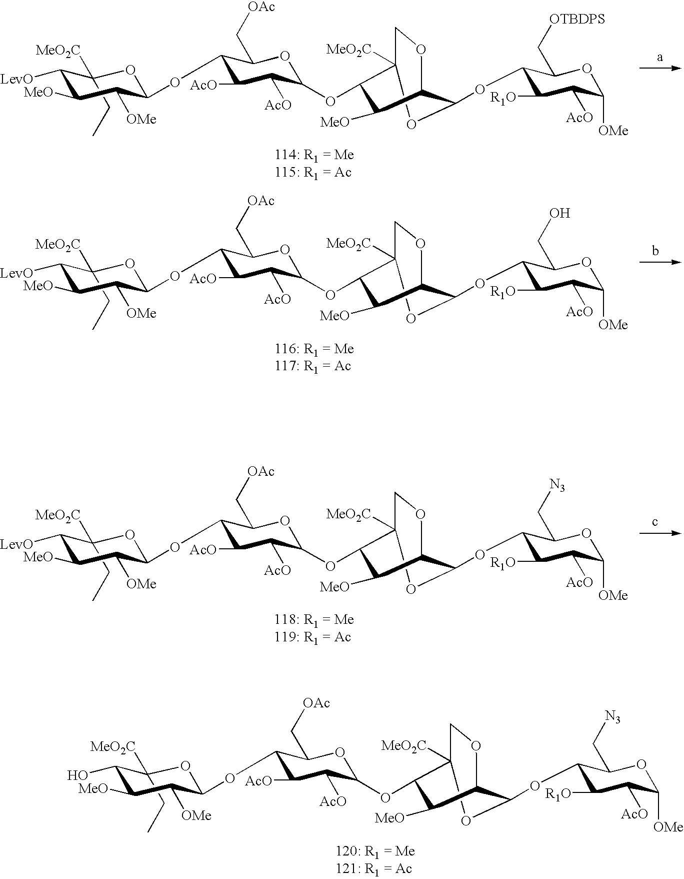

Tetrasaccharides 114 and 115 were prepared using the same procedure that was used for the preparation of tetrasaccharide 107.

### Step 11.a: Synthesis of methyl O-(methyl-4-O-levulinyl-2,3-di-O-methyl-5-C-ethyl-β-D-glucopyranosyluronate)-(1→4)-O-(2,3,6-tri-O-acetyl-α-D-glucopyranosyl)-(1→4)-O-methyl-2,6-anhydro-3-O-methyl-β-D-mannopyranosyluronate)-(1→4)-O-2-O-acetyl-3-O-methyl-α-D-glucopyranoside 116

In a 20 mL round bottom flask, compound 114 (0.5 g, 0.374 mmol) was dissolved in dry pyridine (7.5 mL) and the solution was cooled down to 0° C. Then, hydrogen fluoride pyridine (HF.pyridine) (330 μL, 50 eq.) was added dropwise and the stirring was maintained for 28 h and the reaction temperature was allowed to increase to room temperature. At 0° C., an excess of HF.pyridine complex was quenched by addition of methoxytrimethylsilane (3.2 mL, 1.2 eq/HF.pyridine eq.) and the resultant solution was stirred for 1 h at room temperature. The reaction mixture was concentrated to dryness under vacuum and the resulting residue was purified by silica chromatography (ethyl acetate/heptane: 6/1) to give compound 116 as a white amorphous powder (334 mg, 81%).

¹H NMR (400 MHz, CDCl₃, ppm), δ=5.39 (t, 1H, J_{2,3}=J_{3,4}=9.6 Hz, H-3 Glc^{III}); 5.29 (d, 1H, J_{1,2}=3.5 Hz, H-1 Glc^{III}); 5.23 (sl, 1H, H-1 ManUA^{II}); 5.10 (d, 1H, J_{3,4}=7.6 Hz, H-4 GlcUA^{IV}); 4.88 (d, 1H, J_{1,2}=3.5 Hz, H-1 Glc^{I}); 4.75 (m, 2H, H-2 Glc^{I} and H-2 Glc^{III}); 4.40 (d, 1H, J_{1,2}=8.6 Hz, H-1 GlcUA^{IV}); 3.80 and 3.66 (2s, 6H, CO₂Me); 3.61, 3.52, 3.49, 3.43 and 3.37 (5s, 15H, OMe); 2.75 (m, 2H, CH₂—CH₂—COCH₃); 2.60 (m, 2H, CH₂—CH₂—COCH₃); 2.19 (s, 3H, CH₂—CH₂—COCH₃); 2.16, 2.11, 2.09 and 2.07 (4s, 12H, CH₃—OAc); 1.74 (m, 1H, CH—CH₃); 0.92 (t, 3H, J=7.6 Hz, CH₃—CH).

### Step 11.b: Synthesis of methyl O-(methyl-4-O-levulinyl-2,3-di-O-methyl-5-C-ethyl-β-D-glucopyranosyluronate)-(1→4)-O-(2,3,6-tri-O-acetyl-α-D-glucopyranosyl)-(1→4)-O-(methyl-2,6-anhydro-3-O-methyl-β-D-mannopyranosyluronate)-(1→4)-O-2-O-acetyl-3-O-methyl-6-azido-6-deoxy-α-D-glucopyranoside 118

In a 50 mL round-bottom flask, compound 116 (711 mg, 0.65 mmol) was dissolved in dry pyridine (8 mL). Then, at room temperature, mesyl chloride (75 μL, 0.95 mmol, 1.5 eq.) was added dropwise. After 2 h, the reaction mixture was concentrated under vacuum and the resulting residue was dissolved in dichloromethane. Well known work-up conditions afforded a crude mesylated compound (738 mg) which was used in the next step without any further purification.

In a 50 mL round-bottom flask, intermediate mesylated compound (0.65 mmol) was dissolved in dimethylformamide (16 mL). Sodium azide (420 mg, 6.5 mmol, 10 eq.) was added and the mixture was heated overnight at 55° C. Then, the reaction mixture was filtered and the filtrate was concentrated to dryness under vacuum. Dilution in dichloromethane followed by classical work-up and purification by silica gel chromatography afforded compound 118 as a white amorphous powder (555 mg, 82% over 2 steps).

¹H NMR (400 MHz, CDCl₃, ppm), δ=5.40 (t, 1H, J_{2,3}=J_{3,4}=9.6 Hz, H-3 Glc^{III}); 5.30 (d, 1H, J_{1,2}=3.5 Hz, H-1 Glc^{III}); 5.19 (sl, 1H, H-1 ManUA^{II}); 5.11 (d, 1H, J_{3,4}=8.9 Hz, H-4 GlcUA^{IV}); 4.90 (d, 1H, J_{1,2}=3.4 Hz, H-1 Glc^{I}); 4.77 (m, 2H, H-2 Glc^{I} and H-2 Glc^{III}); 4.40 (d, 1H, J_{1,2}=8.0 Hz, H-1 GlcUA^{IV}); 3.79 and 3.66 (2s, 6H, CO₂Me); 3.63, 3.52, 3.49, 3.44 and 3.41 (5s, 15H, OMe); 2.75 (m, 2H, CH₂—CH₂—COCH₃); 2.59 (m, 2H, CH₂—CH₂—COCH₃); 2.19 (s, 3H, CH₂—CH₂—COCH₃); 2.17, 2.11, 2.10 and 2.08 (s, 12H, CH₃—OAc); 1.74 (m, 1H, CH—CH₃); 0.93 (t, 3H, J=7.6 Hz, CH₃—CH).

### Step 11.c: Synthesis of methyl O-(methyl-2,3-di-O-methyl-5-C-ethyl-β-D-glucopyranosyluronate)-(1→4)-O-(2,3,6-tri-O-acetyl-α-D-glucopyranosyl)-(1→4)-O-(methyl-2,6-anhydro-3-O-methyl-β-D-mannopyranosyluronate)-(1→4)-O-2-O-acetyl-3-O-methyl-6-azido-6-deoxy-α-D-glucopyranoside 120

In a 25 mL round bottom flask, at room temperature, compound 118 (0.595 mg, 0.53 mmol) was dissolved in dichloromethane/methanol (1/2) mixture (5.3 mL) and hydrazine acetate (100 mg, 1 mmol, 2 eq.) was added. The resultant mixture was stirred 3 h at room temperature. Well known work-up conditions followed by purification on silica gel (dichloromethane/ethyl acetate 6/4+1% ethanol) afforded compound 120 as a white amorphous solid (363 mg, 67%).

¹H NMR (400 MHz, CDCl₃, ppm), δ=5.39 (t, 1H, J_{2,3}=J_{3,4}=9.7 Hz, H-3 Glc^{III}); 5.30 (d, 1H, J_{1,2}=3.9 Hz, H-1 Glc^{III}); 5.18 (sl, 1H, H-1 ManUA^{II}); 4.90 (d, 1H, J_{1,2}=3.9 Hz, H-1 Glc^{I}); 4.78 (m, 2H, H-2 Glc^{I} and H-2 Glc^{III}); 4.40 (d, 1H, J_{1,2}=7.8 Hz, H-1 GlcUA^{IV}); 3.79 and 3.78 (2s, 6H, CO₂Me); 3.62, 3.61, 3.53, 3.44 and 3.41 (5s, 15H, OMe); 2.15, 2.11 and 2.08 (4s, 12H, CH₃—OAc); 1.73 (m, 1H, CH—CH₃); 0.96 (t, 3H, J=7.4 Hz, CH₃—CH).

Tetrasaccharide 121 was prepared using the same procedure that was used for the preparation of tetrasaccharide 120.

### Methyl O-(methyl-2,3-di-O-methyl-5-C-ethyl-β-D-glucopyranosyluronate)-(1→4)-O-(2,3,6-tri-O-acetyl-α-D-glucopyranosyl)-(1→4)-O-(methyl-2,6-anhydro-3-O-methyl-β-D-mannopyranosyluronate)-(1→4)-O-2,3-di-O-acetyl-6-azido-6-deoxy-α-D-glucopyranoside 121

¹H NMR (400 MHz, CDCl₃, ppm), δ=5.55 (t, 1H, J_{2,3}=J_{3,4}=9.8 Hz, H-3 Glc^{I}); 5.34 (t, 1H, J_{2,3}=J_{3,4}=9.8 Hz, H-3 Glc^{III}); 5.30 (d, 1H, J_{1,2}=3.5 Hz, H-1 Glc^{III}); 5.01 (sl, 1H, H-1 ManUA^{II}); 4.92 (d, 1H, J_{1,2}=3.5 Hz, H-1 Glc^{I}); 4.86 (dd, 1H, J_{1,2}=3.5 Hz and J_{3,4}=9.8 Hz, H-2 Glc^{I}); 4.79 (dd, 1H, J_{1,2}=3.5 Hz and J_{3,4}=9.8 Hz, H-2 Glc^{III}); 4.39 (d, 1H, J_{1,2}=8.1 Hz, H-1 GlcUA^{IV}); 3.77 and 3.74 (2s, 6H, CO₂Me); 3.61, 3.52, 3.43 (3s, 12H, OMe); 2.10, 2.09, 2.08 and 2.07 (4s, 15H, CH₃—OAc); 1.73 (m, 1H, CH—CH₃); 0.97 (t, 3H, J=7.5 Hz, CH₃—CH).

### Preparation 12: Synthesis of the Tetrasaccharides 126 and 127

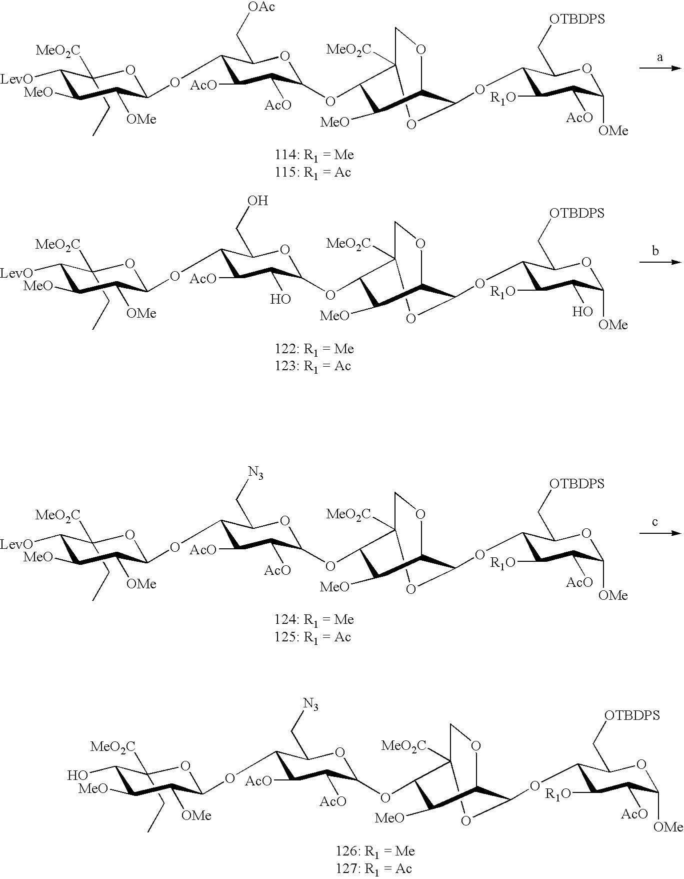

### Step 12.a: Synthesis of methyl O-(methyl-4-O-levulinyl-2,3-di-O-methyl-5-C-ethyl-β-D-glucopyranosyluronate)-(1→4)-O-(2,3-di-O-acetyl-α-D-glucopyranosyl)-(1→4)-O-(methyl-2,6-anhydro-3-O-methyl-β-D-mannopyranosyluronate)-(1→4)-O-2-O-acetyl-3-O-methyl-6-O-tert-butyldiphenylsilyl-α-D-glucopyranoside 122

In a 25 mL round bottom flask, compound 114 (0.9 mg, 0.67 mmol) was dissolved in a tetrahydrofuran/methanol (1/1) mixture (6.8 mL) [tert-BuSnOH(Cl)]₂ (152 mg, 0.27 mmol, 0.4 eq.) was added and, the resulting mixture was heated at 45° C. for 5 h. Concentration of the solvents followed by purification by silica gel chromatography (dichloromethane/ethyl acetate: 8/2+1% ethanol) gave compound 122 as a white amorphous powder (388 mg, 45%)

¹H NMR (400 MHz, CDCl₃, ppm), δ=7.73-7.34 (m, 10H, arom.); 5.35 (m, 2H, H-3 Glc^{III} and H-1 Glc^{III}); 5.17 (sl, 1H, H-1 ManUA^{II}); 5.12 (d, 1H, J_{3,4}=8.8 Hz, H-4 GlcUA^{IV}); 4.90 (d, 1H, J_{1,2}=3.5 Hz, H-1 Glc^{I}); 4.80 (dd, 1H, J_{1,2}=3.5 Hz and J_{3,4}=9.7 Hz, H-2 Glc^{I}); 4.79 (dd, 1H, J_{1,2}=3.8 Hz and J_{3,4}=9.5 Hz, H-2 Glc^{III}); 4.57 (d, 1H, J_{1,2}=7.9 Hz, H-1 GlcUA^{IV}); 3.8 and 3.66 (3s, 6H, CO₂Me); 3.61, 3.51, 3.49, 3.33 and 3.24 (5s, 15H, OMe); 2.76 (m, 2H, CH₂—CH₂—COCH₃); 2.60 (m, 2H, CH₂—CH₂—COCH₃); 2.19 (s, 3H, CH₂—CH₂—COCH₃); 2.18, 2.09 and 2.01 (s, 9H, CH₃—OAc); 1.78 (m, 1H, CH—CH₃); 1.08 (s, 9H, CH₃-tBu); 0.90 (t, 3H, J=8.0 Hz, CH₃—CH).

The next steps i.e. azidation (b) and levulinoyl cleavage (c) were realized as described to get tetrasaccharide 120.

### Methyl O-(methyl-2,3-di-O-methyl-5-C-ethyl-β-D-glucopyranosyluronate)-(1→4)-O-(2,3-di-O-acetyl-6-azido-6-deoxy-α-D-glucopyranosyl)-(1→4)-O-(methyl-2,6-anhydro-3-O-methyl-β-D-mannopyranosyluronate)-(1→4)-O-2-O-acetyl-3-O-methyl-6-O-tert-butyldiphenylsilyl-α-D-glucopyranoside 126

¹H NMR (400 MHz, CDCl₃, ppm), [symbol] =7.74-7.33 (m, 10H, arom.); 5.35 (t, 1H, J_{1,2}=J_{2,3}=10.2 Hz, H-3 Glc^{I}); 5.30 (d, 1H, J_{1,2}=3.6 Hz, H-1 Glc^{I}); 5.16 (sl, 1H, H-1 ManUA^{II}); 4.90 (d, 1H, J_{1,2}=3.6 Hz, H-1 Glc^{I}); 4.79 (m, 2H, H-2 Glc^{I} and H-2 Glc^{III}); 4.41 (d, 1H, J_{1,2}=7.9 Hz, H-1 GlcUA^{IV}); 3.8 and 3.78 (3s, 6H, CO₂Me); 3.62, 3.61, 3.52, 3.34 and 3.27 (5s, 15H, OMe); 2.18, 2.08 and 2.03 (s, 9H, CH₃—OAc); 1.79 (m, 1H, CH—CH₃); 1.07 (s, 9H, CH₃-tBu); 0.98 (t, 3H, J=8.0 Hz, CH₃—CH).

Tetrasaccharide 127 was prepared using the same procedure that was used for the preparation of tetrasaccharide 126.

### Methyl O-(methyl-2,3-di-O-methyl-5-C-ethyl-β-D-glucopyranosyluronate)-(1→4)-O-(2,3-di-O-acetyl-6-azido-6-deoxy-α-D-glucopyranosyl)-(1→4)-O-(methyl-2,6-anhydro-3-O-methyl-β-D-mannopyranosyluronate)-(1→4)-O-2,3-di-O-acetyl-6-O-tert-butyldiphenylsilyl-α-D-ducopyranoside 126

¹H NMR (400 MHz, CDCl₃, ppm), δ=7.73-7.34 (m, 10H, arom.); 5.58 (t, 1H, J_{1,2}=J_{2,3}=9.5 Hz, H-3 Glc^{I}); 5.33 (t, 1H, J_{1,2}=J_{2,3}=9.5 Hz, H-3 Glc^{III}); 5.27 (d, 1H, J_{1,2}=3.7 Hz, H-1 Glc^{I}); 5.00 (sl, 1H, H-1 ManUA^{II}); 4.93 (d, 1H, J_{1,2}=3.6 Hz, H-1 Glc^{I}); 4.86 (dd, 1H, J_{1,2}=3.5 Hz and J_{3,4}=9.5 Hz, H-2 Glc^{I}); 4.77 (dd, 1H, J_{1,2}=3.5 Hz and J_{3,4}=9.5 Hz, H-2 Glc^{III}); 4.40 (d, 1H, J_{1,2}=8.5 Hz, H-1 GlcUA^{IV}); 3.77 and 3.75 (2s, 6H, CO₂Me); 3.62, 3.52, 3.35 and 3.25 (4s, 12H, OMe); 2.12, 2.10, 2.07 and 2.03 (s, 12H, CH₃—OAc); 1.78 (m, 1H, CH—CH₃); 1.09 (s, 9H, CH₃-tBu); 0.97 (t, 3H, J=8.0 Hz, CH₃—CH).

### Preparation of Pentasaccharides

### Preparation 13: Synthesis of Protected Pentasaccharides

Below is reported the general formula of the protected pentasaccharides synthesized.

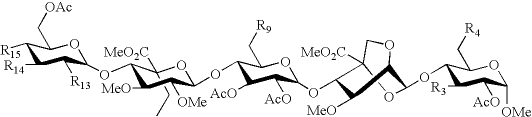

| Compound | R₃ | R₄ | R₉ | R₁₃ | R₁₄/R₁₅ |
|---|---|---|---|---|---|
| 128 | OAc | OAc | OAc | OBn | N₃ |
| 129 | OAc | OTBDPS | OTBDPS | OBn | OBn |
| 130 | OMe | OTBDPS | OTBDPS | OBn | OBn |
| 131 | OMe | OTBDPS | OTBDPS | OMe | OMe |
| 132 | OMe | OTBDPS | OTBDPS | OBu | OBu |
| 133 | OMe | OTBDPS | OTBDPS | OHex | OHex |
| 134 | OMe | OTBDPS | OTBDPS | N₃ | OBn |
| 135 | OMe | OTBDPS | OTBDPS | N₃ | OMe |
| 136 | OMe | OTBDPS | OTBDPS | N₃ | OBu |
| 137 | OMe | OTBDPS | OTBDPS | N₃ | OHex |
| 138 | OAc | N₃ | OAc | OMe | OMe |
| 139 | OAc | OTBDPS | N₃ | OMe | OMe |
| 140 | OAc | N₃ | N₃ | OMe | OMe |
| 141 | OMe | N₃ | OAc | OMe | OMe |
| 142 | OMe | OTBDPS | N₃ | OMe | OMe |
| 143 | OMe | N₃ | OAc | N₃ | OBu |
| 144 | OMe | OTBDPS | N₃ | N₃ | OBu |
| 145 | OMe | OTBDPS | OTBDPS | N₃ | O—(CH₂)₃-Phenyl |

### Synthesis of methyl O-(6-acetyl-2-azido-2-deoxy-3,4-di-O-benzyl-α-D-glucopyranosyl)-(1→4)-O-(methyl-2,3-di-O-methyl-5-C-ethyl-β-D-glucopyranosyluronate)-(1→4)-O-(2,3,6-tri-O-acetyl-α-D-glucopyranosyl)-(1→4)-O-(methyl-2,6-anhydro-3-O-methyl-O-D-mannopyranosyluronate)-(1→4)-2,3,6-tri-O-acetyl-α-D-glucopyranoside 128

Coupling of compound 107 (20 mg, 18.7 μmol.) with compound 49 (17 mg, 1.6 eq.) was performed as described in Method I and gave after purification by preparative TLC (toluene/EtOAc, 2/3) compound 128 (17 mg, 62%).

¹H NMR (400 MHz, CDCl₃, ppm), δ=5.32 (bs, 1H, H-1 Glc^{V}), 5.20 (d, 1H, J=3.6 Hz, H-1 Glc^{III}), 4.85 (bs, 2H, H-1H, H-1 ManUA^{II}), 4.29 (d, 1H, J=12.0 Hz, H-2 GlcUA^{IV}). ESI-MS, positive mode, m/z: 1502.8 [M+Na]⁺, 1518.7 [M+K]⁺

The remaining pentasaccharides were prepared using the same procedure that was used to prepare pentasaccharide 128.

### Methyl O-(6-acetyl-2,3,4-tri-O-benzyl-α-D-glucopyranosyl)-(1→4)-O-(methyl-2,3-di-O-methyl-5-C-ethyl-β-D-glucopyranosyluronate)-(1→4)-O-(2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-2,3-di-O-acetyl-6-O-tert-butyldiphenylsilyl-α-D-glucopyranoside 129

¹H NMR (400 MHz, CDCl₃, ppm), [symbol] =5.23 (d, 1H, J=3.3 Hz, H-1), 4.91 (d, 1H, J=3.4 Hz, H-1 Glc^{I}), 4.53 (d, 1H, J=8.3 Hz, H-1 Glc^{IV}).

### Methyl O-(6-acetyl-2,3,4-tri-O-benzyl-α-D-glucopyranosyl)-(1→4)-O-(methyl-2,3-di-O-methyl-5-C-ethyl-β-D-glucopyranosyluronate)-(1→4)-O-(2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-2-O-acetyl-3-O-methyl-6-O-tert-butyldiphenylsilyl-α-D-glucopyranoside 130

¹H NMR (400 MHz, CDCl₃, ppm): δ=5.33 (s, 1H, H-1 Glc^{III}), 5.20 (d, 1H, J=3.6 Hz, H-1 Glc^{V}), 5.13 (s, 1H, H-1 ManUA^{II}), 4.53 (d, 1H, J=8.7 Hz, H-1 Glc^{IV}).

### Methyl O-(6-acetyl-2,3,4-tri-O-methyl-α-D-glucopyranosyl)-(1→4)-O-(methyl-2,3-di-O-methyl-5-C-ethyl-β-D-glucopyranosyluronate)-(1→4)-O-(2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-2-O-acetyl-3-O-methyl-6-O-tert-butyldiphenylsilyl-α-D-glucopyranoside 131

¹H NMR (400 MHz, CDCl₃, ppm), δ=5.32 (s, H-1, H-1 Glc^{III}), 5.19 (d, 1H, J=3.8 Hz, H-1 Glc^{V}), 5.11 (s, 1H, H-1 ManUA^{II}), 4.86 (d, 1H, J=3.3 Hz, H-1 Glc^{I}), 4.49 (d, 1H, J=8.3 Hz, H-1 Glc^{IV}).

MALDI, m/z: 1704.11 [M+Na]⁺, 1719.03 [M+K]⁺

[α]_{D}=57.95 (c=0.0055, CHCl₃)

### Methyl O-(6-acetyl-2,3,4-tri-O-butyl-α-D-glucopyranosyl)-(1→4)-O-(methyl-2,3-di-O-methyl-5-C-ethyl-β-D-glucopyranosyl uronate)-(1→4)-O-(2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-2-O-acetyl-3-O-methyl-6-O-tert-butyldiphenylsilyl-α-D-glucopyranoside 132

¹H NMR (400 MHz, CDCl₃, ppm), δ=5.40-5.30 (m, 2H, H-1 Glc^{III}, Glc^{V}) 5.12 (d, 1H, H-1 ManUA^{II}), 4.89 (d, 1H, J=3.4 Hz, H-1 Glc^{I}), 4.51 (d, 1H, J=8.3 Hz, H-1 Glc^{IV}).

MALDI, m/z: 1829.96 [M+Na]⁺, 1845.92 [M+K]⁺

[α]_{D}=66.4 (c=0.0041, CHCl₃)

### Methyl O-(6-acetyl-2,3,4-tri-O-hexyl-α-D-glucopyranosyl)-(1→4)-O-(methyl-2,3-di-O-methyl-5-C-ethyl-β-D-glucopyranosyluronate)-(1→4)-O-(2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-2-O-acetyl-3-O-methyl-6-O-tert-butyldiphenylsil-α-D-glucopyranoside 133

¹H NMR (400 MHz, CDCl₃, ppm), δ=5.34 (d, 1H, J=3.2 Hz, H-1 Glc^{III}), 5.20 (d, 1H, J=3.4 Hz, H-1 Glc^{V}), 5.13 (s, 1H, H-1 ManUA^{II}), 4.89 (d, 1H, J=3.5 Hz, H-1 Glc^{I}), 4.53 (d, 1H, J=8.0 Hz, H-1 Glc^{IV})

[α]_{D}=69 (c=0.0046, CHCl₃)

### Methyl O-(6-acetyl-2-azido-2-deoxy-3,4-di-O-benzyl-α-D-glucopyranosyl)-(1→4)-O-(methyl-2,3-di-O-methyl-5-C-ethyl-β-D-glucopyranosyluronate)-(1→4)-O-(2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-2-O-acetyl-3-O-methyl-6-O-tert-butyldiphenylsilyl-α-D-glucopyranoside 134

¹H NMR (400 MHz, CDCl₃, ppm), δ=5.34 (d, 1H, J=3.6 Hz, H-1 Glc^{V}), 5.32 (d, 1H, J=3.4 Hz, H-1 Glc^{III}), 5.11 (s, 1H, H-1 ManUA^{II}), 4.86 (d, 1H, J=3.6 Hz, H-1 Glc^{I}), 4.50 (d, 1H, J=8.0 Hz, H-1 Glc^{IV}).

MALDI, 1883.87 [M+Na]⁺, 1867.94 [M+K]⁺

[α]_{D}=68 (c=0.003, CHCl₃)

### Methyl O-(6-acetyl-2-azido-2-deoxy-3,4-di-O-methyl-α-D-glucopyranosyl)-(1→4)-O-(methyl-2,3-di-O-methyl-5-C-ethyl-β-D-glucopyranosyluronate)-(1→4)-2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-2-O-acetyl-3-O-methyl-6-O-tert-butyldiphenylsilyl-α-D-glucopyranoside 135

¹H NMR (400 MHz, CDCl₃, ppm), δ=5.39-5.32 (m, 2H, H-1, H-3 Glc^{III}), 5.30 (d, 1H, J=3.6 Hz, H-1 Glc^{V}), 5.12 (s, 1H, H-1 ManUA^{II}), 4.88 (d, 1H, J=3.6 Hz, H-1 Glc^{I}), 4.51 (d, 1H, J=8.4 Hz, H-1 Glc^{IV}).

### Methyl O-(6-acetyl-2-azido-2-deoxy-3,4-di-O-butyl-α-D-glucopyranosyl)-(1→4)-O-(methyl-2,3-di-O-methyl-5-C-ethyl-β-D-glucopyranosyluronate)-(1→4)-(2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-2-O-acetyl-3-O-methyl-6-O-tert-butyldiphenylsilyl-α-D-glucopyranoside 136

¹H NMR (400 MHz, CDCl₃, ppm), δ=5.33-5.25 (m, H-1, H-3 Glc^{III}), 5.23 (d, 1H, J=3.8 Hz, H-1 Glc^{V}), 5.05 (s, 1H, H-1 ManUA^{II}), 4.80 (d, 1H, J=3.4 Hz, H-1 Glc^{I}), 4.51 (d, 1H, J=8.3 Hz, H-1 Glc^{IV}).

### Methyl O-(6-acetyl-2-azido-2-deoxy-3,4-di-O-hexyl-α-D-glucopyranosyl)-(1→4)-O-(methyl-2,3-di-O-methyl-5-C-ethyl-β-D-glucopyranosyluronate)-(1→4)—O-(2,3-di-O-acetyl-6-O-tert-butyldiphenylsilyl-α-D-glucopyranosyl)-(1→4)-O-(methyl-2,6-anhydro-3-O-methyl-β-D-mannopyranosyluronate)-(1→4)-2-O-acetyl-3-O-methyl-6-O-tent-butyldiphenylsilyl-α-D-glucopyranoside 137

¹H NMR (400 MHz, CDCl₃, ppm), δ=5.33 (d 1H, J=2.8 Hz, H-1 Glc^{III}), 5.30 (d, 1H, J=3.8 Hz, H-1 Glc^{V}), 5.12 (s, 1H, H-1 ManUA^{II}), 4.88 (d, 1H, J=3.3 Hz, H-1 Glc^{I}), 4.53 (d, 1H, J=8.4 Hz, H-1 Glc^{IV}).

### Methyl O-(6-acetyl-2-azido-2,3,4-tri-O-methyl-α-D-glucopyranosyl)-(1→4)-O-methyl-2,3-di-O-methyl-5-C-ethyl-β-D-glucopyranosyluronate)-(1→4)-O-(2,3,6-tri-O-acetyl-α-D-glucopyranosyl)-(1→4)-O-(methyl-2,6-anhydro-3-O-methyl-β-D-mannopyranosyluronate)-(1→4)-2,3-di-O-acetyl-6-azido-6-deoxy-α-D-glucopyranoside 138

¹H NMR (400 MHz, CDCl₃, ppm), δ=5.19 (d 1H, J=3.4 Hz, H-1 Glc^{III}), 5.14 (d, 1H, J=3.6 Hz, H-1 Glc^{V}), 4.92 (s, 1H, H-1 ManUA^{II}), 4.84 (d, 1H, J=3.5 Hz, H-1 Glc^{I}), 4.44 (d, 1H, J=7.9 Hz, H-1 Glc^{IV})

MALDI, m/z: 1322.43 [M+Na]⁺, 1338.31 [M+k]⁺

[α]_{D}=92.9 (c=0.75, CHCl₃)

### Methyl O-(6-acetyl-2-azido-2,3,4-tri-O-methyl-α-D-glucopyranosyl)-(1→4)-O-(methyl-2,3-di-O-methyl-5-C-ethyl-β-D-glucopyranosyluronate)-(1→4)-O-(2,3-di-O-acetyl-6-azido-6-deoxy-α-D-glucopyranosyl)-(1→4)-O-(methyl-2,6-anhydro-3-O-methyl-β-D-mannopyranosyluronate)-(1→4)-2,3-di-O-acetyl-6-O-tert-butyldimethylsilyl-α-D-glucopyranoside 139

¹H NMR (400 MHz, CDCl₃, ppm), δ=5.20 (d 1H, J=3.7 Hz, H-1 Glc^{III}), 5.10 (d, 1H, J=3.5 Hz, H-1 Glc^{V}), 4.92 (s, 1H, H-1 ManUA^{II}), 4.85 (d, 1H, J=3.6 Hz, H-1 Glc^{I}), 4.36 (d, 1H, J=8.0 Hz, H-1 Glc^{IV})

MALDI, m/z: 1518.48 [M+Na]⁺, 1534.39 [M+K]⁺

[α]_{D}=90.7 (c=0.76, CHCl₃)

### Methyl O-(6-acetyl-2,3,4-tri-O-methyl-α-D-glucopyranosyl)-(1→4)-O-(methyl-2,3-di-O-methyl-5-C-ethyl-β-D-glucopyranosyluronate)-(1→4)-O-(2,3-di-O-acetyl-6-azido-6-deoxy-α-D-glucopyranosyl)-(1→4)-O-(methyl-2,6-anhydro-3-O-methyl-β-D-mannopyranosyluronate)-(1→4)-2,3-di-O-acetyl-6-azido-6-deoxy-α-D-glucopyranoside 140

¹H NMR (400 MHz, CDCl₃, ppm), δ=5.38-5.28 (m, 2H, H-1, H-3 Glc^{III}), 5.17 (d, 1H, J=3.4 Hz, H-1 Glc^{V}), 5.01 (s, 1H, H-1 ManUA^{II}), 4.92 (d, 1H, J=3.0 Hz, H-1 Glc^{I}), 4.43 (d, 1H, J=8.0 Hz, H-1 Glc^{IV})

MALDI, m/z: 1305.71 [M+Na]⁺, 1321.61 [M+K]⁺

[α]_{D}=108 (c=1.318, CHCl₃)

### Methyl O-(6-acetyl-2,3,4-tri-O-methyl-α-D-glucopyranosyl)-(1→4)-O-(methyl-2,3-di-O-methyl-5-C-ethyl-β-D-glucopyranosyluronate)-(1→4)-O-(2,3,6-tri-O-acetyl-α-D-glucopyranosyl)-(1→4)-O-(methyl-2,6-anhydro-3-O-methyl-β-D-mannopyranosyluronate)-(1→4)-2-O-acetyl-3-O-methyl-6-azido-6-deoxy-α-D-glucopyranoside 141

¹H NMR (400 MHz, CDCl₃, ppm), δ=5.29 (d, 1H, J=3.3 Hz, H-1 Glc^{III}), 5.22 (d, 1H, J=3.8 Hz, H-1 Glc^{V}), 5.18 (s, 1H, H-1 ManUA^{II}), 4.89 (d, 1H, J=3.8 Hz, H-1 Glc^{I}), 4.49 (d, 1H, J=8.0 Hz, H-1 Glc^{IV}).

MALDI, m/z: 1294.61 [M+Na]⁺, 1310.52 [M+K]⁺

### Methyl O-(6-acetyl-2,3,4-tri-O-methyl-α-D-glucopyranosyl)-(1→4)-O-(methyl-2,3-di-O-methyl-5-C-ethyl-β-D-glucopyranosyluronate)-(1→4)-O-(2,3-di-O-acetyl-6-azido-6-deoxy-α-D-glucopyranosyl)-(1→4)-O-(methyl-2,6-anhydro-3-O-methyl-β-D-mannopyranosyluronate)-(1→4)-2-O-acetyl-3-O-methyl-6-O-tert-butyldimethylsilyl-α-D-glucopyranoside 142

¹H NMR (400 MHz, CDCl₃, ppm), δ=5.33-5.28 (m, 2H, H-1 Glc^{III}, H-1 Glc^{V}), 5.16 (s, 1H, H-1 ManUA^{II}), 4.90 (d, 1H, J=3.5 Hz, H-1 GlcI), 4.40 (d, 1H, J=7.4 Hz, H-1 GlcIV).

### Methyl O-(6-acetyl-2-azido-2-deoxy-3,4-di-O-methyl-α-D-glucopyranosyl)-(1→4)-O-(methyl-2,3-di-O-methyl-5-C-ethyl-β-D-glucopyranosyluronate)-(1→4)-O-(2,3,6-tri-O-acetyl-α-D-glucopyranosyl)-(1→4)-O-(methyl-2,6-anhydro-3-O-methyl-β-D-mannopyranosyluronate)-(1→4)-2-O-acetyl-3-O-methyl-6-azido-6-deoxy-α-D-glucopyranoside 143

¹H NMR (400 MHz, CDCl₃, ppm), δ=5.37 (d 1H, J=3.7 Hz, H-1 Glc^{III}), 5.34 (d, 1H, J=3.7 Hz, H-1 Glc^{V}), 5.22 (s, 1H, H-1 ManUA^{II}), 4.94 (d, 1H, J=3.4 Hz, H-1 Glc^{I}), 4.40 (d, 1H, J=8.2 Hz, H-1 Glc^{IV})

MALDI, m/z: 1597.27 [M+Na]⁺, 1614.07 [M+K]⁺

### Methyl O-(6-acetyl-2-azido-2-deoxy-3,4-di-O-methyl-α-D-glucopyranosyl)-(1→4)-O-(methyl-2,3-di-O-methyl-5-C-ethyl-β-D-glucopyranosyluronate)-(1→4)-O-(2,3-di-O-acetyl-6-azido-6-deoxy-α-D-glucopyranosyl)-(1→4)-O-(methyl-2,6-anhydro-3-O-methyl-β-D-mannopyranosyluronate)-(1→4)-2-O-acetyl-3-O-methyl-6-O-tert-butyldiphenylsilyl-α-D-glucopyranoside 144

¹H NMR (400 MHz, CDCl₃ ppm), δ=5.37-5.26 (m, 3H), 5.15 (s, 1H, H-1 ManUA^{II}), 4.90 (d, 1H, J=3.6 Hz, H-1 Glc^{I}), 4.39 (d, 1H, J=8.2 Hz, H-1 Glc^{IV})

[α]_{D}=117 (c=1, CHCl₃).

### Methyl O-(2-azido-2-deoxy-3,4-di-O-(3-phenylpropyl)-6-O-acetyl-α-glucopyranosyl)-(1→4)-O-(methyl-2,3-di-O-methyl-5-C-ethyl-β-D-glucopyranosyluronate)-(1→4)-O-(2,3-di-O-acetyl-α-D-glucopyranosyl)-(1→4)-O-(methyl-2,6-anhydro-3-O-methyl-β-D-mannopyranosyluronate)-(1→4)-O-2-O-acetyl-3-O-methyl-α-D-glucopyranoside 145

¹H NMR (400 MHz, CDCl₃, ppm), δ: 5.37-5.32 (m, 2H, H-1 Glc^{III}, Glc^{V}), 5.14 (s, 1H, H-1 ManUA^{II}), 4.90 (d, 1H, J=3.4 Hz, H-1 Glc^{I}), 4.53 (d, 1H, J=8.11 Hz, H-1 Glc^{IV}).

MALDI, m/z: 1924.01 [M+Na]⁺, 1939.96 [M+K]⁺.

[α]_{D}=137 (c=1, CHCl₃).

### Preparation 14: Synthesis of Sulphated Pentasaccharides

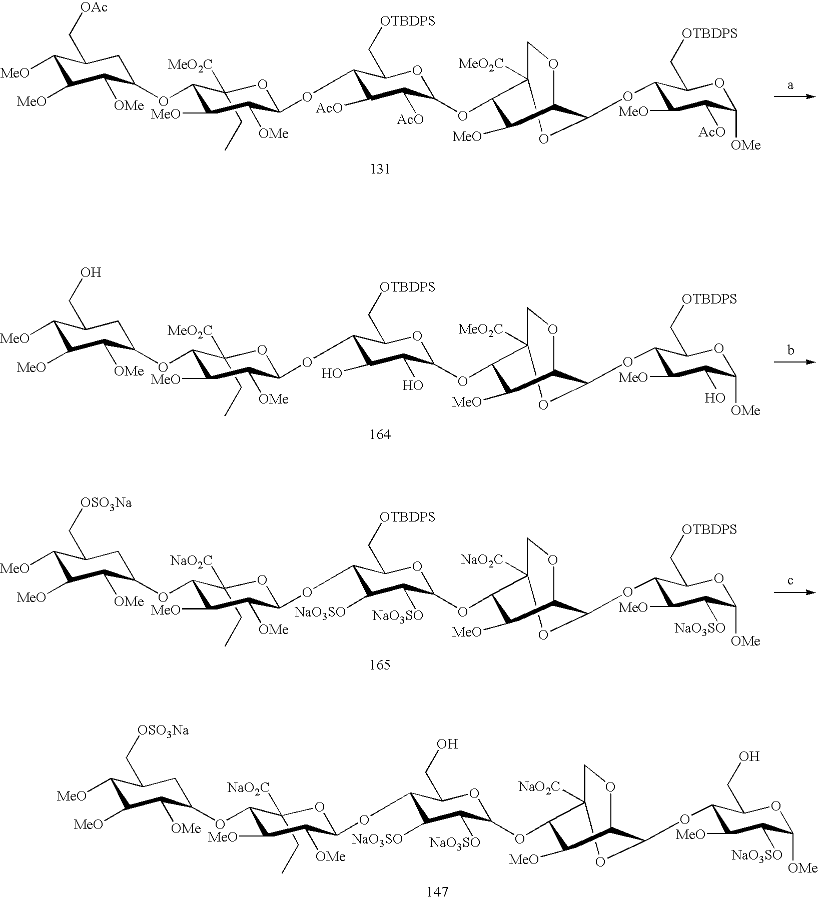

### Step 14.a: synthesis of methyl O-(2,3,4-tri-O-methyl-α-D-glucopyranosyl)-(1→4)-O-(methyl-2,3-di-O-methyl-5-C-ethyl-β-D-glucopyranosyluronate)-(1→4)-O-(6-O-tert-butyldiphenylsilyl-α-D-glucopyranosyl)-(1→4)-O-(methyl-2,6-anhydro-3-O-methyl-β-D-mannopyranosyluronate)-(1→4)-3-O-methyl-6-O-tert-butyldiphenylsilyl-α-D-glucopyranoside 164

Pentasaccharide 131 was treated according to Method J. Purification on a sephadex LH-20 column gave compound 164 (85%)

ESI-MS, negative mode, ink: 741.51 [M−2H]²⁻.

### Step 14. b: Synthesis of methyl O-(2,3,4-tri-O-methyl-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-6-O-tert-butyldiphenylsilyl-α-D-glucopyranosyl)-(1→4)-O-(2,6-anhydro-3-O-methyl-β-D-mannopyranosyluronate)-(1→4)-O-2-O-sulfo-3-O-methyl-6-O-tert-butyldiphenylsilyl-α-D-glucopyranoside, hexasodium salt 165

Pentasaccharide 164 was treated according to Method K, which gave, after purification on a sephadex LH-20 column eluted with DMF, compound 165 (96%).

¹H NMR (400 MHz, MeOD ppm), δ=5.43 (d 1H, J=3.4 Hz, H-1 Glc^{III}), 5.33 (d, 1H, J=3.8 Hz, H-1 Glc^{V}), 4.96 (d, 1H, J=3.6 Hz, H-1 Glc^{I}), 4.77 (s, 1H, H-1 ManUA^{II}), 4.67 (d, 1H, J=8.5 Hz, H-1 Glc^{IV}).

ESI-MS, negative mode, m/z: 1031.12 [M+2DBA−4H]²⁻, 966.53 [M+DBA−3H]²⁻, 901.95 [M−2H]²⁻, 608.3 [M−3H]³⁻.

### Step 14.c: Synthesis of methyl O-(2,3,4-tri-O-methyl-6-O-sulfo-α-D-glucopyranosyl)-(1→4)—O-(2,3-di-O-methyl-5-C-ethyl-β-D-glucopyranosyluronate)-(1→4)-O-(2,3-di-O-sulfo-α-D-glucopyranosyl)-(1→4)-O-(2,6-anhydro-3-O-methyl-β-D-mannopyranosyluronate)-(1→4)-O-2-O-sulfo-3-O-methyl-α-D-glucopyranoside, hexasodium salt 147

Pentasaccharide 165 was treated according to Method L, which gave, after purification on a sephadex LH-20 column, compound 147 (86%).

¹H NMR (400 MHz, MeOD ppm), δ=5.43 (d 1H, J=3.8 Hz, H-1 Glc^{III}), 5.12 (d, 1H, J=3.3 Hz, H-1 Glc^{V}), 5.03 (s, 1H, H-1 ManUA^{II}), 4.96-4.91 (m, 2H, H-1 Glc^{I}, Glc^{IV}).

ESI-MS, negative mode, m/z: 1039.61 [M+3DBA−5H]²⁻, 975.01 [M+2DBA−4H]²⁻, 655.61 [M+2DBA−5H]³⁻, 563.20 [M−3H]³⁻.

Below is the general formula of the sulphated pentasaccharides synthesized. The remaining compounds described below were obtained in a similar procedure to that of used to obtain pentasaccharide 147.

### Family 1: R₃═OMe

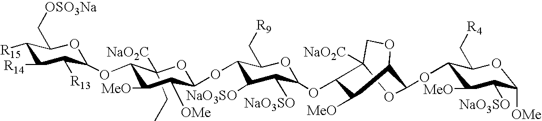

| Compound | R₄ | R₉ | R₁₃ | R₁₄/R₁₅ |
|---|---|---|---|---|
| 146 | OH | OH | OBn | OBn |
| 147 | OH | OH | OMe | OMe |
| 148 | OH | OH | OBu | OBu |
| 149 | OH | OH | OHex | OHex |
| 150 | OH | OH | N₃ | OBn |
| 151 | OH | OH | N₃ | OMe |
| 152 | OH | OH | N₃ | OBu |
| 153 | OH | OH | N₃ | OHex |
| 154 | N₃ | OSO₃Na | OMe | OMe |
| 155 | OSO₃Na | N₃ | OMe | OMe |
| 156 | N₃ | OSO₃Na | N₃ | OBu |
| 157 | OSO₃Na | N₃ | N₃ | OBu |
| 158 | OH | OH | N₃ | O—(CH₂)₃-Phenyl |

### Methyl O-(2,3,4-tri-O-benzyl-6-O-sulfo-α-D-glucopyranosyl)-(1→4)-O-(2,3-di-O-methyl-5-C-ethyl-β-D-glucopyranosyluronate)-(1→)-O-(2,3-di-O-sulfo-α-D-glucopyranosyl)-(1→4)-O-(2,6-anhydro-3-O-methyl-β-D-mannopyranosyluronate)-(1→4)-O-2-O-sulfo-3-O-methyl-α-D-glucopyranoside, hexasodium salt 146

¹H NMR (400 MHz, MeOD, ppm), δ=5.40 (d 1H, J=3.3 Hz, H-1 Glc^{III}), 5.13 (d, 1H, J=3.0 Hz, H-1 Glc^{V}), 5.01 (s, 1H, H-1 ManUA^{II}), 4.96-4.90 (m, 2H, H-1 Glc^{I}, Glc^{IV}).

ESI-MS, negative mode, m/z: 1835.96 [M+2DBA−2H−Na]¹⁻, 1729.75 [M+2DBA−3H−SO₃]¹⁻, 1852.09 [M+3DBA−4H]¹⁻.

### Methyl O-(2,3,4-tri-O-butyl-6-O-sulfo-α-D-glucopyranosyl)-(1—>4)—O-(2,3-di-O-methyl-5-C-ethyl-β-D-glucopyranosyluronate)-(1→4)-O-(2,3-di-O-sulfo-α-D-glucopyranosyl)-(1→4)-O-(2,6-anhydro-3-O-methyl-β-D-mannopyranosyluronate)-(1→4)-O-2-O-sulfo-3-O-methyl-α-D-glucopyranoside, hexasodium salt 148

¹H NMR (400 MHz, MeOD ppm), δ=5.41 (d 1H, J=3.0 Hz, H-1 Glc^{III}), 5.35 (br, 1H, H-1 Glc^{V}), 4.94 (d, 1H, J=3.3 Hz, H-1 Glc^{I}), 4.70-4.58 (m, 1H, H-1 Glc^{IV}).

ESI-MS, negative mode, m/z: 1136.35 [M+2DBA−4H]²⁻, 1071.73 [M+DBA−3H]²⁻, 671.04 [M−3H]³⁻.

### Methyl O-(2,3,4-tri-O-hexyl-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-α-D-glucopyranosyl)-(1→4)-O-(2,6-anhydro-3-O-methyl-β-D-mannopyranosyluronate)-(1→4)-O-2-O-sulfo-3-O-methyl-α-D-glucopyranoside, hexasodium salt 149

¹H NMR (400 MHz, MeoD ppm), δ=5.40 (d 1H, J=3.8 Hz, H-1 Glc^{III}), 5.02-4.98 (m, 2H, H-1 ManUA^{II}, Glc^{V}), 4.92-4.81 (m, 2H, H-1, Glc^{I}, Glc^{IV}).

ESI-MS, negative mode, m/z: 897.67 [M+2DBA−4H]²⁻, 833.07 [M+DBA−3H]²⁻, 768.44 [M-3H]³⁻, 511.97 [M−3H]³⁻.

### Methyl O-(2-azido-2-deoxy-3,4-di-O-benzyl-6-O-sulfo-α-D-glucopyranosyl)-(1→4)-O-(2,3-di-O-methyl-5-C-ethyl-β-D-glucopyranosyluronate)-(1→4)-(2,3-di-O-sulfo-α-D-glucopyranosyl)-(1→4)-O-(2,6-anhydro-3-O-methyl-β-D-mannopyranosyluronate)-(1→4)-O-2-O-sulfo-3-O-methyl-α-D-glucopyranoside, hexasodium salt 150

¹H NMR (400 MHz, MeOD ppm), δ=5.42 (d 1H, J=3.7 Hz, H-1 Glc^{III}), 5.32 (d, 1H, J=3.7 Hz, H-1 Glc^{V}), 5.01 (s, 1H, H-1 ManUA^{II}), 4.93 (d, 1H, J=3.7 Hz, H-1 Glc^{I}), 4.75 (d, 1H, H-1 Glc^{IV}).

### Methyl O-(2-azido-2-deoxy-3,4-di-O-methyl-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-α-D-glucopyranosyl)-(1→4)-O-(2,6-anhydro-3-O-methyl-β-D-mannopyranosyluronate)-(1→4)-O-2-O-sulfo-3-O-methyl-α-D-glucopyranoside, hexasodium salt 151

¹H NMR (400 MHz, MeOD ppm), δ=5.39 (d 1H, J=3.7 Hz, H-1 Glc^{III}), 5.20 (d, 1H, J=3.7 Hz, H-1 Glc^{V}), 4.99 (s, 1H, H-1 ManUA^{II}), 4.90 (d, 1H, J=3.3 Hz, H-1 Glc^{I}), 4.72 (d, 1H, J=7.8 Hz, H-1 Glc^{IV}).

ESI-MS, negative mode, m/z: 798.03 [M+2DBA−5H]²⁻, 733.42 [M+DBA−3H]²⁻, 668.81 [M+2H]²⁻, 498.96 [M−3H]³⁻.

### Methyl O-(2-azido-2-deoxy-3,4-di-O-butyl-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-α-D-glucopyranosyl)-(1→4)-O-(2,6-anhydro-3-O-methyl-β-D-mannopyranosyluronate)-(1→4)-O-2-O-sulfo-3-O-methyl-α-D-glucopyranoside, hexasodium salt 152

¹H NMR (400 MHz, MeOD, ppm), δ=5.40 (d 1H, J=3.4 Hz, H-1 Glc^{III}), 5.20 (d, 1H, J=2.9 Hz, H-1 Glc^{V}), 5.0 (s, 1H, H-1 ManUA^{II}), 4.91 (d, 1H, J=3.8 Hz, H-1 Glc^{I}), 4.73 (d, 1H, H-1 Glc^{IV}).

ESI-MS, negative mode, 117m/z: 840.08 [M+2DBA−4H]²⁻, 775.48 [M+DBA−3H]²⁻, 710.87 [M+2H]²⁻, 473.56 [M−3H]³.

### Methyl O-(2-azido-2-deoxy-3,4-di-O-hexyl-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-α-D-glucopyranosyl)-(1→4)-O-(2,6-anhydro-3-O-methyl-β-D-mannopyranosyluronate)-(1→4)-O-2-O-sulfo-3-O-methyl-α-D-glucopyranoside, hexasodium salt 153

¹H NMR (400 MHz, MeOD, ppm), δ=5.40 (d 1H, J=3.7 Hz, H-1 Glc^{III}), 5.20 (d, 1H, J=3.7 Hz, H-1 Glc^{V}), 5.0 (s, 1H, H-1 ManUA^{II}), 4.91 (d, 1H, J=3.4 Hz, H-1 Glc^{I}), 4.71 (d, 1H, H-1 Glc^{IV}).

ESI-MS, negative mode, m/z: 868.2 [M+2DBA−4H]²⁻, 803.6 [M+DBA−3H]²⁻, 738.9 [M−2H]²⁻.

### Methyl O-(2,3,4-tri-O-methyl-6-O-sulfo-α-D-glucopyranosyl)-(1→4)-O-(2,3-di-O-methyl-5-C-ethyl-β-D-glucopyranosyluronate)-(1→4)-O-(2,3,6-tri-O-sulfo-α-D-glucopyranosyl)-(1→4)-O-(2,6-anhydro-3-O-methyl-βD-mannopyranosyluronate)-(1→4)-O-2-O-sulfo-3-O-methyl-6-azido-6-deoxy-α-D-glucopyranoside, heptasodium salt 154

¹H NMR (400 MHz, D₂O, ppm), δ=5.47 (d 1H, J=3.6 Hz, H-1 Glc^{III}), 5.41 (d, 1H, J=3.6 Hz, H-1 Glc^{V}), 5.24 (s, 1H, H-1 ManUA^{II}), 5.07 (d, 1H, J=3.8 Hz, H-1 Glc^{I}), 4.66 (d, 1H, J=7.9 Hz, H-1 Glc^{IV}).

ESI-MS, negative mode, m/z: 845.1 [M+2DBA−4H]²⁻, 476.9 [M−3H]³⁻.

### Methyl O-(2,3,4-tri-O-methyl-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-6-azido-6-deoxy-α-D-glucopyranosyl)-(1→4)-O-(2,6-anhydro-3-O-methyl-β-D-mannopyranosyluronate)-(1→4)-O-2,6-di-O-sulfo-3-O-methyl-α-D-glucopyranoside, heptasodium salt 155

¹H NMR (400 MHz, MeOD, ppm), δ=5.46 (d, 1H, J=3.4 Hz, H-1, GlcIII), 5.14 (s, 1H, H-1 ManUA^{II}), 5.07 (b 1H, H-1 Glc^{V}), 4.93 (d, 1H, J=3.3 Hz, H-1 Glc^{I}), 4.62 (d, 1H, J=7.6 Hz, H-1 Glc^{IV}).

### Methyl O-(2-azido-2-deoxy-3,4-di-O-butyl-6-O-sulfo-α-D-glucopyranosyl)-(1→4)-O-(2,3-di-O-methyl-5-C-ethyl-β-D-glucopyranosyluronate)-(1→4)-O-(2,3,6-tri-O-sulfo-α-D-glucopyranosyl)-(1→4)-O-(2,6-anhydro-3-O-methyl-β-D-mannopyranosyluronate)-(1→4)-O-2-O-sulfo-3-O-methyl-6-azido-6-deoxy-α-D-glucopyranoside, heptasodium salt 156

¹H NMR (400 MHz, MeOD, ppm), δ=5.48 (d 1H, J=3.5 Hz, H-1 Glc^{III}), 5.10 (d, 1H, J=3.7 Hz, H-1 Glc^{V}), 5.24 (s, 1H, H-1 ManUA^{II}), 5.08 (d, 1H, J=3.7 Hz, H-1 Glc^{I}), 4.67 (d, 1H, J=8.0 Hz, H-1 Glc^{IV}).

ESI-MS, negative mode, m/z: 957.3 [M+3DBA−5H]²⁻, 892.7 [M+2DBA−4H]²⁻, 828.1 [M+DBA−3H]²⁻, 508.6 [M−3H]³⁻.

### Methyl O-(2-azido-2-deoxy-3,4-di-O-butyl-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-6-azido-6-deoxy-α-D-glucopyranosyl)-(1→4)-O-(2,6-anhydro-3-O-methyl-β-D-mannopyranosyluronate)-(1→4)-O-2,6-di-O-sulfo-3-O-methyl-α-D-glucopyranoside, heptasodium salt 157

¹H NMR (400 MHz, MeOD, ppm), δ=5.46 (d, 1H, J=3.5 Hz, H-1, Glc^{III}), 5.11 (d, 1H, J=3.7 Hz, H-1 Glc^{V}), 5.14 (s, 1H, H-1 ManUA^{II}), 4.91 (d, 1H, J=3.4 Hz, H-1 Glc^{I}), 4.58 (d, 1H, J=8.5 Hz, H-1 Glc^{IV}).

### Methyl O-(2-azido-2-deoxy-3,4-di-O-(3-phenylpropyl)-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-α-D-glucopyranosyl)-(1→4)-O-(2,6-anhydro-3-O-methyl-β-D-mannopyranosyluronate)-(1→4)-O-2-O-sulfo-3-O-methyl-α-D-glucopyranoside, hexasodium salt 158

¹H NMR (400 MHz, D₂O, ppm), δ=5.41 (d, 1H, J=3.8 Hz, H-1), 5.05-4.99 (m, 2H, H-1), 4.92 (d, 1H, J=3.5 Hz, H-1), 4.80 (d, 1H, J=3.7 Hz, H-1).

ESI-MS, negative mode, m/z: 902.2 [M−2DBA−4H]²⁻, 837.6 [M+DBA−3H]²⁻, 773.0 [M−2H]²⁻.

### Family 2: R₃═OSO₃Na

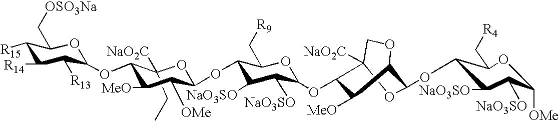

| Compound | R₄ | R₉ | R₁₃ | R₁₄/R₁₅ |
|---|---|---|---|---|
| 159 | OH | OH | OBn | OBn |
| 160 | OSO₃Na | OSO₃Na | OBn | N₃ |
| 161 | N₃ | OSO₃Na | OMe | OMe |
| 162 | OSO₃Na | N₃ | OMe | OMe |
| 163 | N₃ | N₃ | OMe | OMe |

### Methyl O-(2,3,4-tri-O-benzyl-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-α-D-glucopyranosyl)-(1→4)-O-(2,6-anhydro-3-O-methyl-β-D-mannopyranosyluronate)-(1→4)-O-2,3-di-O-sulfo-α-D-glucopyranoside, heptasodium salt 159

¹H NMR (400 MHz, MeOD, ppm), δ=5.43 (d 1H, J=3.07 Hz, H-1 Glc^{III}), 5.30 (s, 1H, H-1 ManUA^{II}), 5.15 (d, 1H, J=3.07 Hz, H-1 Glc^{V}), 4.99-4.90 (m, 2H, H-1 Glc^{I}, Glc^{IV}).

ESI-MS, negative mode, m/z: 1003.5 [M+3DBA−5H]²⁻, 625.8 [M+2DBA−5H]²⁻, 582.8 M+2DBA−5H]²⁻.

### Methyl O-(2-azido-2-deoxy-3,4-di-O-benzyl-6-O-sulfo-α-D-glucopyranosyl)-(1→4)-O-(2,3-di-O-methyl-5-C-ethyl-β-D-glucopyranosyluronate)-(1→4)-O-(2,3,6-tri-O-sulfo-α-D-glucopyranosyl)-(1→4)-O-(2,6-anhydro-3-O-methyl-β-D-mannopyranosyluronate)-(1→4)-O-2,3,6-tri-O-sulfo-α-D-glucopyranoside, nonasodium salt 160

¹H NMR (400 MHz, D₂O, ppm), δ=5.43 (bs, 2H, H-1 Glc^{III}, H-1 ManUA^{II}), 5.22 (d, 1H, J=3.6 Hz, H-1 Glc^{V}), 5.04 (d, 1H, J=3.6 Hz, H-1 Glc^{I}), 4.88 (d, 1H, J=7.5 Hz, H-1GlcUA^{IV}), 4.67 (m, 1H, H-3 Glc^{III}), 4.46 (m, 1H, H-3 Glc^{I}), 4.25 (m, 2H, H-2 Glc^{III}, H-2 Glc^{I}).

ESI-MS, negative mode, m/z: 1051.4 [M+3DBA−5H]²⁻, 986.8 [M+2DBA−4H]²⁻, 946.8 [M+1DBA−4H]²⁻.

### Methyl O-(2,3,4-tri-O-methyl-6-O-sulfo-α-D-glucopyranosyl)-(1→4)-O-(2,3-di-O-methyl-5-C-ethyl-β-D-glucopyranosyluronate)-(1→4)-O-(2,3,6-tri-O-sulfo-α-D-glucopyranosyl)-(1→4)-O-(2,6-anhydro-3-O-methyl-β-D-mannopyranosyluronate)-(1→4)-O-2,3-di-O-sulfo-6-azido-6-deoxy-α-D-glucopyranoside, octasodium salt 161

¹H NMR (400 MHz, D₂O, ppm), δ=5.47-5.42 (m, 2H, H-1, Glc^{III}, ManUA^{II}) 5.38 (d, 1H, J=3.8 Hz, H-1 Glc^{V}), 5.12 (d, 1H, J=3.6 Hz, H-1 Glc^{I}), 4.62 (d, 1H, J=8.7 Hz, H-1 Glc^{IV}).

ESI-MS, negative mode, m/z: 942.66 [M+3DBA−5H]²⁻, 878.05 [M+2DBA−4H]²⁻, 946.8 [M+1DBA−4H]²⁻.

### Methyl O-(2,3,4-tri-O-methyl-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-6-azido-6-deoxy-α-D-glucopyranosyl)-(1→4)-O-(2,6-anhydro-3-O-methyl-β-D-mannopyranosyluronate)-(1→4)-O-2,3,6-tri-O-sulfo-α-D-glucopyranoside, octasodium salt 162

¹H NMR (400 MHz, D₂O, ppm), δ=5.26 (d 1H, J=3.9 Hz, H-1 Glc^{III}), 5.22 (s, 1H, H-1 ManUA^{II}), 5.10 (d, 1H, J=3.9 Hz, H-1 Glc^{V}), 4.90 (d, 1H, J=3.7 Hz, H-1 Glc^{I}), 4.42 (d, 1H, J=8.2 Hz, H-1 Glc^{IV}).

ESI-MS, negative mode, m/z: 942.6 [M+3DBA−5H]²⁻, 878.0 [M+2DBA−4H]²⁻.

### Methyl O-(2,3,4-tri-O-methyl-6-O-sulfo-α-D-glucopyranosyl)-(1→4)-O-(2,3-di-O-methyl-5-C-ethyl-β-D-glucopyranosyluronate)-(1→4)-O-(2,3-di-O-sulfo-6-azido-6-deoxy-α-D-glucopyranosyl)-(1→4)-O-(2,6-anhydro-3-O-methyl-β-D-mannopyranosyluronate)-(1→4)-O-2,3-di-O-sulfo-6-azido-6-deoxy-α-D-glucopyranoside, octasodium salt 163

¹H NMR (400 MHz, MeOD, ppm), δ=5.54 (d 1H, J=3.7 Hz, H-1 Glc^{III}), 5.40 (s, 1H, H-1 ManUA^{II}), 5.16 (d, 1H, J=3.1 Hz, H-1 Glc^{V}), 5.08 (d, 1H, J=3.5 Hz, H-1 Glc^{I}), 4.73 (d, 1H, J=7.4 Hz, H-1 Glc^{IV}).

ESI-MS, negative mode, m/z: 850.55 [M+2DBA−4H]²⁻, 785.94 [M+2DBA−3H]²⁻, 732.34 [M−2H]²⁻, 480.56 [M−3H]³⁻.

### EXAMPLES

### General Methods

Method O: General Method for Acylation with a Succinimide Reagent

A succinimide reagent (1.5 molar equivalents/NH₂ group) and a solution of diisopropylethylamine 0.2M/DMF (1.5 molar equivalent/NH₂ group) was added to a solution of pentasaccharide (1 molar equivalent) in anhydrous DMF (100 L/mol). The mixture was stirred at room temperature for 24 h. After this time, a saturated aqueous solution of NaHCO₃ was added to the reaction mixture (25 L/pentasaccharide mol). After the resultant mixture was stirred at room temperature for 16 h, it was filtered and poured onto either a Sephadex LH-20 column (320 mL) equilibrated with DMF, or onto a Sephadex G25F column (3 L/mmol, 0.2 N NaCl). The combined fractions were concentrated and desalted on a Sephadex G25F column (water) to give the acylated pentasaccharide.

Method P: General Method for Acylation with an Anhydride Reagent

Triethylamine (1.5 molar equivalents) and an anhydride reagent (1.2 molar equivalents) was added to a solution of pentasaccharide (1 molar equivalent) in anhydrous DMF (100 L/mol) that was cooled at 0° C. After the mixture was stirred at room temperature for 20 h, a 0.1M aqueous solution of NaOH (66 L/pentasaccharide mol) was added and the resultant mixture was stirred at room temperature for a further 16 h. It was then filtered and either directly poured onto a Sephadex LH-20 column (320 mL) equilibrated with DMF, or poured onto a Sephadex G25F column (3 L/mmol, 0.2 N NaCl). The combined fractions were concentrated and desalted on a Sephadex G25F column (water) to the give the acylated pentasaccharide.

A similar reaction can be performed in a pyridine/anhydride mixture.

Method Q: General Method for Acylation with an Acyl Chloride Reagent

Triethylamine (10 molar equivalents) and an acyl chloride reagent (5 molar equivalents) were added to a solution of pentasaccharide (1 molar equivalent) in anhydrous DMF (100 L/mol). After the mixture was stirred at room temperature for 20 h, a saturated aqueous solution of NaHCO₃ was added (30 L/pentasaccharide mol). The mixture was then stirred at room temperature for a further 16 h. It was then filtered and the solution was either directly poured onto a Sephadex LH-20 column (320 mL) equilibrated with DMF, or poured onto a Sephadex G25F column (3 L/mmol, 0.2 N NaCl). The combined fractions were concentrated and desalted on a Sephadex G25F column (water) to the give the acylated pentasaccharide.

### Method R: General Method for Alkylation and Saponification

NaH 60%/oil (5 molar equivalents/OH) was added to a solution of pentasaccharide (1 molar equivalent) in DMF (100 L/mol) at 0° C. After the mixture was stirred for 10 min, an alkylating agent (15 molar equivalents) was added and the solution was stirred at room temperature for a further 16 h. It was then neutralized with methanol, stirred for 2 h and directly poured onto a Sephadex LH-20 column (320 mL) equilibrated with DMF to give the alkylated and esterified product.

The resultant compound was then dissolved in a methanol/THF mixture (ratio 1:2, 150 L/pentasaccharide mol) and a 2M aqueous solution of KOH (50 L/pentasaccharide mol) was added dropwise. After the mixture was stirred at room temperature for 48 h, a saturated aqueous solution of NaHCO₃ was added (100 L/pentasaccharide mol). The mixture was then stirred at room temperature for a further 16 h. It was then filtered and the solution was directly poured onto a Sephadex LH-20 column (320 mL) equilibrated with DMF to the give the alkylated and saponified pentasaccharide.

### Method S: General Method for Sulphation

A sulfur trioxide pyridine complex (5 molar eq./OH) was added to a solution of pentasaccharide (1 molar equivalent) in anhydrous pyridine (77 L/mol). The mixture was heated at 80° C. with protection from light for 16 h. After cooling to 0° C., the solution was neutralized with methanol (40 molar eq./PyrSO₃) and stirred for 2 h. After this time, a saturated aqueous solution of NaHCO₃ was added (30 L/pentasaccharide mol). The mixture was then stirred at room temperature for a further 16 h. It was then filtered and the solution was either directly poured onto a Sephadex LH-20 column (320 mL) equilibrated with DMF, or poured onto a Sephadex G25F column (3 L/mmol, 0.2 N NaCl). The combined fractions were concentrated and desalted on a Sephadex G25F column (water) to the give the sulfated pentasaccharide.

### Method T: General Method for Hydrogenolysis

A solution of pentasaccharide (1 molar equivalent) in 1:1 tert-butanol/water mixture (0.1 mL/mg) was stirred under hydrogen in the presence of Pd(OH)₂/C catalyst (20%, 0.5 weight equivalent) for 48 h and filtered through Celite® 45 and PTFE millipore membrane. The solution was concentrated to dryness to give the hydrogenolysed product.

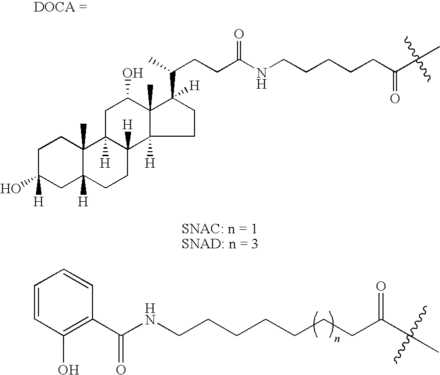

### O-Alkyl/Family: R₁₃═R₁₄/R¹⁵

Compounds Derived from 4S Templates

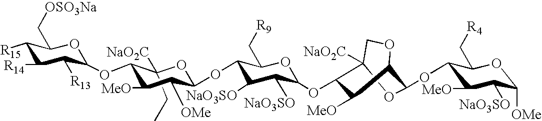

| Example | R₁₃/R₁₄/R₁₅ | R₉ | R₄ |
|---|---|---|---|
| 1 | OH | Odecanoyl | Odecanoyl |
| 2 | OBn | Odecanoyl | Odecanoyl |
| 3 | OBn | OAc | OAc |
| 4 | OBn | OMe | OMe |
| 5 | OBn | Ooctyl | Ooctyl |
| 6 | OBn | OH | OH |
| 7 | OBu | OH | OH |
| 8 | OBn | OSO₃Na | OSO₃Na |
| 9 | OMe | OSO₃Na | N₃ |
| 10 | OMe | OSO₃Na | NH(3-cyclopentylpropanoyl) |
| 11 | OMe | OSO₃Na | NH(3,5- |
| | | | bis(trifluoromethyl)benzoyl) |
| 12 | OMe | OSO₃Na | NHDOCA |
| 13 | OMe | OSO₃Na | NHSNAD |
| 14 | OMe | OSO₃Na | NH(Z-aminohexanoyl) |
| 15 | OMe | OSO₃Na | NHhexanoyl |
| 16 | OMe | OSO₃Na | NHhydrocinnamoyl |
| 17 | OMe | N₃ | OSO₃Na |
| 18 | OMe | NHDOCA | OSO₃Na |

### Example 1

This example was prepared from example 2 according to Method T (yield: 39%).

¹H NMR (400 MHz, CD₃OD, ppm), δ: 5.46-5.38 (broad s, 1H, H-1), 5.00-4.84 (m, 3H, H-1).

ESI-MS, negative mode, m/z: 925.7 [M+2DBA−4H]²⁻, 861.1 [M+DBA−3H]²⁻, 796.5 [M−2H]²⁻

### Example 2

This example was prepared according to Method P (yield: 91%).

¹H NMR (400 MHz, CD₃OD, ppm), δ: 5.40 (d, 1H, J=3.6 Hz, H-1 Glc^{III}), 5.18 (d, 1H, J=3.4 Hz, H-1 Glc^{V}), 4.96 (s, 1H, H-1 ManUA^{II}), 4.92 (d, 1H, J=3.4 Hz, H-1 Glc^{I}), 4.77 (1H, H-1 Glc^{IV}).

ESI-MS, negative mode, m/z: 1061.2 [M+2DBA−4H]²⁻, 996.6 [M+DBA−3H]²⁻.

### Example 3

This example was prepared according to Method P (yield: 85%).

¹H NMR (400 MHz, CD₃OD, ppm), δ: 5.39-5.34 (braod s, 1H, H-1), 5.15 (d, 1H, J=3.2 Hz, H-1), 5.00 (s, 1H, H-1), 4.94 4.87 (m, 2H, H-1), 4.72 (1H, H-1).

ESI-MS, negative mode, m/z: 1061.2 [M+2DBA−4H]²⁻, 996.6 [M+DBA−3H]²⁻.

### Example 4

This example was prepared according to Method R (yield: 90%).

¹H NMR (400 MHz, CD₃OD, ppm), δ: 5.42 (d, 1H, J=3.4 Hz, H-1 Glc^{III}), 5.18 (d, 1H, J=2.7 Hz, H-1 Glc^{V}), 4.99 (s, 1H, H-1 ManUA^{II}), 4.93 (d, 1H, J=3.3 Hz, H-1 Glc^{I}), 4.77 (d, 1H, H-1 Glc^{IV}).

ESI-MS, negative mode, m/z: 856.0 [M+DBA−3H]²⁻, 791.4 [M−2H]²⁻, 743.5 [M+5DBA−8H]³⁻.

### Example 5

This example was prepared according to Method R (yield: 92%).

ESI-MS, negative mode, m/z: 1019.5 [M+2DBA−4H]²⁻, 954.4 [M+DBA−3H]²⁻.

### Example 6

This example was prepared according to preparation 14 (compound 145).

### Example 7

This example was prepared according to preparation 14 (compound 147).

### Example 8

This example was prepared according to Method S (yield: 80%).

¹H NMR (400 MHz, CD₃OD, ppm), δ: 5.44 (d, 1H, J=3.6 Hz, H-1 Glc^{III}), 5.42 (d, 1H, J=3.3 Hz, H-1 Glc^{V}), 5.07 (s, 1H, H-1 ManUA^{II}), 4.89 (d, 1H, J=3.7 Hz, H-1 Glc^{I}), 4.71 (d, 1H, J=8.5 Hz, H-1 Glc^{IV}).

ESI-MS, negative mode, m/z: 1051.1 [M+3DBA−5H]²⁻, 986.5 [M+2DBA−4H]²⁻.

### Example 9

This example was prepared according to preparation 14 (compound 154).

### Example 10

This example was prepared according to Method Q (yield: 58%).

¹H NMR (400 MHz, CD₃OD, ppm), δ: 5.43 (d, 1H, J=3.7 Hz, H-1), 5.33 (d, 1H, J=3.7 Hz, H-1), 4.94 (s, 1H, H-1), 4.89 (d, 1H, J=3.4 Hz, H-1).

ESI-MS, negative mode, m/z: 894.2 [M+2DBA−4H]²⁻, 829.6 [M+3DBA−5H]²⁻.

### Example 11

This example was prepared according to Method Q (yield: 86%).

¹H NMR (400 MHz, D₂O, ppm), δ: 5.43 (d, 1H, J=3.5 Hz, H-1 Glc^{III}), 4.69 (d, 1H, J=3.7 Hz, H-1 Glc^{V}), 5.04 (s, 1H, H-1 ManUA^{II}), 4.91 (d, 1H, J=3.4 Hz, H-1 Glc^{I}), 4.69 (d, 1H, J=8.0 Hz, H-1 Glc^{IV}).

ESI-MS, negative mode, m/z: 952.2 [M+2DBA−4H]²⁻, 887.6 [M+DBA−3H]²⁻.

### Example 12

This example was prepared according to Method O (yield=93%).

¹H NMR (400 MHz, CD₃OD, ppm), δ: 5.43 (d, 1H, J=3.5 Hz, H-1 Glc^{III}), 5.33 (d, 1H, J=3.7 Hz, H-1 Glc^{V}), 4.92 (s, 1H, H-1 ManUA^{II}), 4.89 (d, 1H, J=3.6 Hz, H-1 Glc^{I}), 4.69 (d, 1H, J=8.0 Hz, H-1 Glc^{IV}).

ESI-MS, negative mode, m/z: 673.8 [M+DBA−4H]³⁻, 630.8 [M−3H]³⁻.

### Example 13

This example was prepared according to Method O (yield=93%).

¹H NMR (400 MHz, CD₃OD, ppm), δ: 5.43 (d, 1H, J=3.2 Hz, H-1 Glc^{III}), 5.33 (d, 1H, J=3.6 Hz, H-1 Glc^{V}), 4.94 (s, 1H, H-1 ManUA^{II}), 4.89 (d, 1H, J=3.4 Hz, H-1 Glc^{I}), 4.77 (d, 1H, J=7.7 Hz, H-1 Glc^{IV}).

ESI-MS, negative mode, m/z: 607.7 [M+DBA−4H]³⁻, 564.6 [M−3H]³⁻.

### Example 14

This example was prepared according to Method O (yield=68%).

¹H NMR (400 MHz, CD₃OD, ppm), δ: 5.43 (d, 1H, J=3.8 Hz, H-1), 5.33 (d, 1H, J=3.8 Hz, H-1), 4.95 (s, 1H, H-1 ManUA^{II}), 4.89 (d, 1H, J=3.6 Hz, H-1), 4.71 (d, 1H, H-1).

ESI-MS, negative mode, m/z: 955.6 [M+3DBA−5H]²⁻, 550.6 [M−3H]³⁻.

### Example 15

This example was prepared according to Method P (yield=88%).

¹H NMR (400 MHz, CD₃OD, ppm), δ: 5.43 (d, 1H, J=3.3 Hz, H-1 Glc^{III}), 5.33 (d, 1H, J=3.7 Hz, H-1 Glc^{V}), 4.94 (s, 1H, H-1 ManUA^{II}), 4.89 (d, 1H, J=3.5 Hz, H-1 Glc^{I}), 4.70 (1H, H-1 Glc^{IV}).

ESI-MS, negative mode, m/z: 881.1 [M+2DBA−4H]²⁻, 816.5 [M+DBA−3H]²⁻, 500.9 [M−3H]³⁻.

### Example 16

This example was prepared according to Method Q (yield=90%).

¹H NMR (400 MHz, CD₃OD, ppm), δ: 5.43 (d, 1H, J=3.5 Hz, H-1 Glc^{III}), 5.33 (d, 1H, J=3.7 Hz, H-1 Glc^{V}), 4.94 (s, 1H, H-1 ManUA^{II}), 4.86 (d, 1H, J=3.8 Hz, H-1).

ESI-MS, negative mode, m/z: 881.1 [M+2DBA−4H]²⁻, 816.5 [M+DBA−3H]²⁻, 500.9 [M−3H]³⁻.

### Example 17

This example was prepared according to preparation 14 (compound 157).

### Example 18

This example was prepared according to Method O (yield=94%).

¹H NMR (400 MHz, D₂O, ppm), δ: 5.52 (d, 1H, J=3.4 Hz, H-1), 5.32 (d, 1H, J=3.2 Hz, H-1), 5.23 (s, 1H, J=1.0 Hz, H-1 ManUA^{II}), 5.09 (d, 1H, J=3.5 Hz, H-1), 4.70 (d, 1H, J=7.6 Hz, H-1).

ESI-MS, negative mode, m/z: 1140.9 [M+3DBA−5H]²⁻, 1075.8 [M+2DBA−4H]²⁻.

Compounds Derived from 5S Templates

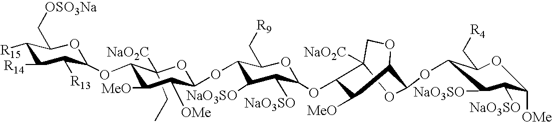

| Example | R₁₃/R₁₄/R₁₅ | R₉ | R₄ |
|---|---|---|---|
| 19 | OBn | OAc | OAc |
| 20 | OBn | Ohexanoyl | Ohexanoyl |
| 21 | OBn | OBn | OBn |
| 22 | OBn | OMe | OMe |
| 23 | OBn | OEt | OEt |
| 24 | OBn | OBu | OBu |
| 25 | OBn | Ohexyl | Ohexyl |
| 26 | OBn | O-(3-phenylpropyl) | O-(3-phenylpropyl) |
| 27 | OBn | Ooctyl | Ooctyl |
| 28 | OMe | O-(4,4,4-trifluorobutyl) | O-(4,4,4-trifluorobutyl) |
| 29 | OBn | OH | OH |
| 30 | OMe | N₃ | N₃ |
| 31 | OMe | NH₂ | NH₂ |
| 32 | OMe | NHDOCA | NHDOCA |
| 33 | OMe | NHSNAD | NHSNAD |
| 34 | OMe | NH(3,5- | NH(3,5- |
| | | bis(trifluoromethyl)benzoyl) | bis(trifluoromethyl)benzoyl) |
| 35 | OMe | NH(4-nitrooxy)butanoyl | NH(4-nitrooxy)butanoyl |
| 36 | OMe | OH | N₃ |
| 37 | OMe | OSO₃Na | N₃ |
| 38 | OMe | OSO₃Na | NH₂ |
| 39 | OMe | OSO₃Na | NHhexanoyl |
| 40 | OMe | OSO₃Na | NHDOCA |
| 41 | OMe | OSO₃Na | NHdodecanoyl |
| 42 | OMe | OSO₃Na | NH(3,5- |
| | | | bis(trifluoromethyl)benzol) |
| 43 | OMe | OSO₃Na | NH(3- |
| | | | cyclopentylpropanoyl) |
| 44 | OMe | OSO₃Na | NH(Z-aminohexanoyl) |
| 45 | OMe | OSO₃Na | NHSNAC |
| 46 | OMe | OSO₃Na | NHoleyl |
| 47 | OMe | OSO₃Na | NH(3-phenylpropanoyl) |
| 48 | OMe | OSO₃Na | NHarachidoyl |
| 49 | OMe | OSO₃Na | NHniflumic |
| 50 | OMe | OSO₃Na | NH(4-nitrooxy)butanoyl |
| 51 | OMe | N₃ | OH |
| 52 | OMe | N₃ | OSO₃Na |
| 53 | OMe | NHDOCA | OSO₃Na |
| 54 | OMe | NHSNAD | OSO₃Na |
| 55 | OMe | NH(3,5- | OSO₃Na |
| | | bis(trifluoromethyl)benzoyl) | |
| 56 | OMe | NHhydrocinnamoyl | OSO₃Na |
| 57 | OMe | NH(Z-aminohexanoyl) | OSO₃Na |
| 58 | OMe | NH(3- | OSO₃Na |
| | | cyclopentylpropanoyl) | |
| 59 | OMe | NHhexanoyl | OSO₃Na |
| 60 | OMe | NH(aminohexanoyl) | OSO₃Na |

### Example 19

This example was prepared according to Method P (yield: 76%).

¹H NMR (400 MHz, CD₃OD, ppm), δ: 5.42 (d, 1H, J=3.4 Hz, H-1), 5.08 (d, 1H, J=7.6 Hz, H-1), 5.02 (s, 1H, H-1), 4.92 (d, 1H, J=2.9 Hz, H-1), 4.82 (d, 1H, J=3.2 Hz, H-1).

ESI-MS, negative mode, m/z: 1045.7 [M+3DBA−5H]²⁻.

### Example 20

This example was prepared according to Method P (yield: 73%).

¹H NMR (400 MHz, CD₃OD, ppm), δ: 5.36 (d, 1H, J=2.7 Hz, H-1), 5.32 (s, 1H, H-1), 5.16 (d, 1H, J=2.5 Hz, H-1), 4.91 (d, 1H, J=2.7 Hz, H-1), 4.71 (d, 1H, H-1).

ESI-MS, negative mode, m/z: 1101.7 [M+3DBA−5H]²⁻, 691.4 [M+2DBA−5H]³⁻.

### Example 21

This example was prepared according to Method R (yield: 65%).

¹H NMR (400 MHz, CD₃OD, ppm), δ: 5.35 (d, 1H, J=3.4 Hz, H-1), 5.27 (s, 1H, H-1), 5.18 (d, 1H, J=3.0 Hz, H-1), 4.95 (d, 1H, J=3.7 Hz, H-1).

ESI-MS, negative mode, m/z: 1030.1 [M+2DBA−4H]²⁻, 965.0 [M+DBA−3H]²⁻.

### Example 22

This example was prepared according to Method R (yield: 63%).

¹H NMR (400 MHz, CD₃OD, ppm), δ: 5.29 (d, 1H, J=7.6 Hz, H-1), 5.16 (broad s, 1H, H-1).

ESI-MS, negative mode, m/z: 953.6 [M+2DBA−4H]²⁻, 889.0 [M+DBA−3H]²⁻.

### Example 23

This example was prepared according to Method R (yield: 53%).

¹H NMR (400 MHz, CD₃OD, ppm), δ: 5.38 (broad s, 1H, H-1), 5.25 (s, 1H, H-1 ManUA^{II}), 5.18-5.11 (m, 1H, H-1), 4.92 (d, 1H, J=3.8 Hz, H-1), 4.65 (broad s, 1H, H-1).

ESI-MS, negative mode, m/z: 967.5 [M+2DBA−4H]²⁻, 902.9 [M+DBA−3H]²⁻.

### Example 24

This example was prepared according to Method R (yield: 28%).

¹H NMR (400 MHz, CD₃OD, ppm), δ: 5.38 (bs, 1H, H-1), 5.25 (s, 1H, H-1 ManUA^{II}), 5.18-5.11 (m, 1H, H-1), 4.92 (d, 1H, J=3.8 Hz, H-1), 4.65 (broad s, 1H, H-1).

ESI-MS, negative mode, m/z: 995.6 [M+2DBA−4H]²⁻, 931.0 [M+DBA−3H]²⁻.

### Example 25

This example was prepared according to Method R (yield: 74%).

¹H NMR (400 MHz, CD₃OD, ppm), δ: 5.42 (d, 1H, J=3.6 Hz, H-1 Glc^{III}), 5.30 (s, 1H, H-1 ManUA^{II}), 5.19 (d, 1H, J=3.0 Hz, H-1), 4.94 (d, 1H, J=3.0 Hz, H-1), 4.71-4.65 (m, 1H, H-1).

ESI-MS, negative mode, m/z: 1023.7 [M+2DBA−4H]²⁻, 959.0 [M+DBA−3H]²⁻.

### Example 26

This example was: prepared according to Method R (yield: 38%).

¹H NMR (400 MHz, CD₃OD, ppm), δ: 5.42 (d, 1H, J=3.7 Hz, H-1), 5.17 (d, 1H, J=2.9 Hz, H-1), 4.95 (d, 1H, J=3.7 Hz, H-1).

ESI-MS, negative mode, m/z: 1057.8 [M+2DBA−4H]²⁻, 993.2 [M+DBA−3H]²⁻.

### Example 27

This example was prepared according to Method R (yield: 5%).

ESI-MS, negative mode, m/z: 1052.3 [M+2DBA−4H]²⁻, 992.8 [M+DBA−3H]²⁻, 714.7 [M−3H]³⁻.

### Example 28

This example was prepared according to Method R (yield: 83%).

¹H NMR (400 MHz, CD₃OD, ppm), δ: 5.43 (d, 1H, J=3.6 Hz, H-1 Glc^{III}), 5.15 (d, 1H, J=3.4 Hz, H-1), 4.98 (s, 1H, H-1 ManUA^{II}), 4.90 (d, 1H, J=3.4 Hz, H-1), 4.66 (d, 1H, J=7.7 Hz, H-1 Glc^{IV}).

ESI-MS, negative mode, m/z: 902.7 [M+2DBA−4H]²⁻, 838.1 [M+DBA−3H]²⁻, 773.5 [M−2H]²⁻.

### Example 29

This example was prepared according to preparation 14 (compound 158).

### Example 30

This example was prepared according to preparation 14 (compound 163).

### Example 31

This example was prepared from example 30 according to Method T (yield: 94%).

¹H NMR (400 MHz, D₂O, ppm), δ: 5.46-5.40 (m, 2H, H-1 Glc^{III}, H-1 ManUA^{II}), 5.31 (d, 1H, J=3.5 Hz, H-1 Glc^{V}), 5.12 (d, 1H, J=3.3 Hz, H-1 Glc^{I}), 4.62 (d, 1H, J=7.8 Hz, H-1 Glc^{IV}).

ESI-MS, negative mode, m/z: 695.3 [M−2H]²⁻.

### Example 32

This example was prepared according to Method O (yield=76%).

¹H NMR (400 MHz, D₂O, ppm), δ: 5.53-5.47 (m, 2H, H-1), 5.38 (bs, 1H, H-1), 5.12 (s, 1H, H-1), 4.63-4.48 (m, 1H, H-1).

ESI-MS, negative mode, m/z: 831.7 [M+DBA−4H]³⁻, 788.6 [M−3H]³⁻.

### Example 33

This example was prepared according to Method I (yield=93%).

¹H NMR (400 MHz, D₂O, ppm), δ: 5.49 (s, 1H, H-1), 5.41 (d, 1H, J=3.6 Hz, H-1), 5.36 (d, 1H, J=3.6 Hz, H-1), 5.11 (d, 1H, J=3.6 Hz, H-1), 4.60-4.55 (m, 1H, H-1 Glc^{IV}).

ESI-MS, negative mode, m/z: 1114.3 [M+2DBA−4H]²⁻, 656.4 [M−3H]³⁻.

### Example 34

This example was prepared according to Method Q (yield=86%).

¹H NMR (400 MHz, D₂O, ppm), δ: 5.50 (s, 1H, H-1), 5.44 (d, 1H, J=3.4 Hz, H-1), 5.38 (d, 1H, J=3.6 Hz, H-1), 5.11 (d, 1H, J=3.6 Hz, H-1).

ESI-MS, negative mode, m/z: 1064.7 [M+2DBA−4H]²⁻, 1000.1 [M+DBA−3H]²⁻, 623.3 [M−3H]³⁻.

### Example 35

This example was prepared according to Method O (yield: 93%).

¹H NMR (400 MHz, D₂O, ppm), δ: 5.43 (bs, 1H, H-1), 5.36 (d, 1H, J=3.2 Hz, H-1), 3.09 (d, 1H, J=3.8 Hz, H-1).

ESI-MS, negative mode, m/z: 955.7 [M+2DBA−4H]²⁻, 891.1 [M+DBA−3H]²⁻.

### Example 36

This example was prepared according to Method H (yield: 73%).

¹H NMR (400 MHz, D₂O, ppm), δ: 5.51 (d, 1H, J=3.8 Hz, H-1 Glc^{III}), 5.49 (s, 1H, H-1 ManUA^{II}), 5.42 (d, 1H, J=3.5 Hz, H-1 Glc^{V}), 5.20 (d, 1H, J=3.6 Hz, H-1 Glc^{I}), 4.87 (d, 1H, J=8.4 Hz, H-1 Glc^{IV}).

ESI-MS, negative mode, m/z: 838.0 [M+2DBA−4H]²⁻, 773.4 [M+DBA−3H]²⁻, 708.8 [M−2H]²⁻, 515.3 [M+DBA−4H]³⁻, 472.3 [M−3H]³⁻.

### Example 37

This example was prepared according to preparation 14 (compound 161).

### Example 38

This example was prepared according to Method T (yield: 93%).

¹H NMR (400 MHz, D₂O, ppm), δ: 5.44 (s, 1H, H-1 ManUA^{II}), 5.40 (d, 1H, J=3.3 Hz, H-1), 5.34 (d, 1H, J=3.6 Hz, H-1), 5.11 (d, 1H, J=3.4 Hz, H-1), 4.60 (d, 1H, J=7.9 Hz, H-1 Glc^{IV}).

ESI-MS, negative mode, m/z: 865.1 [M+2DBA−4H]²⁻, 800.5 [M+DBA−3H]²⁻.

### Example 39

This example was prepared according to Method P (yield: 73%).

¹H NMR (400 MHz, D₂O, ppm), δ: 5.36-5.32 (m, 2H, H-1 Glc^{III}, H-1 ManUA^{II}), 5.27 (d, 1H, J=3.6 Hz, H-1 Glc^{V}), 4.97 (d, 1H, J=3.7 Hz, H-1 Glc^{I}), 4.52 (d, 1H, J=8.2 Hz, H-1 Glc^{IV}).

ESI-MS, negative mode, m/z: 978.6 [M+3DBA−5H]²⁻, 914.1 [M+2DBA−4H]²⁻.

### Example 40

This example was prepared according to Method O (yield: 84%).

¹H NMR (400 MHz, D₂O, ppm), δ: 5.53-5.47 (m, 2H, H-1 Glc^{III}, H-1 ManUA^{II}), 5.44 (d, 1H, J=3.5 Hz, H-1 Glc^{V}), 5.13 (d, 1H, J=3.5 Hz, H-1 Glc^{I}), 4.69 (d, 1H, J=8.0 Hz, H-1 Glc^{IV}).

ESI-MS, negative mode, m/z: 1238.6 [M+4DBA−6H]²⁻, 1174.0 [M+3DBA−5H]²⁻.

### Example 41

This example was prepared according to Method P (yield: 81%).

¹H NMR (400 MHz, D₂O, ppm), δ: 5.55-5.47 (m, 2H, H-1 Glc^{III}, H-1 ManUA^{II}), 5.44 (d, 1H, J=3.8 Hz, H-1 Glc^{V}), 5.14 (d, 1H, J=3.4 Hz, H-1 Glc^{I}), 4.70 (d, 1H, J=8.2 Hz, H-1 Glc^{IV}).

ESI-MS, negative mode, m/z: 1020.8 [M+3DBA−5H]²⁻, 956.2 [M+2DBA−4H]²⁻, 551.0 [M−3H]³⁻.

### Example 42

This example was prepared according to Method Q (yield: 91%).

¹H NMR (400 MHz, D₂O, ppm), δ: 5.54 (s, 1H, H-1 ManUA^{II}), 5.44 (d, 1H, J=3.6 Hz, H-1 Glc^{V}), 5.16 (d, 1H, J=3.4 Hz, H-1 Glc^{I}), 4.69 (d, 1H, J=7.9 Hz, H-1 Glc^{IV}), 5.52-5.49 (m, 1H, H-1 Glc^{III}).

ESI-MS, negative mode, m/z: 1049.7 [M+3DBA−5H]²⁻, 985.1 [M+2DBA−4H]²⁻.

### Example 43

This example was prepared according to Method Q (yield: 73%).

¹H NMR (400 MHz, D₂O, ppm), δ: 5.53-5.48 (m, 2H, H-1 ManUA^{II}, H-1 Glc^{III}), 5.44 (d, 1H, J=3.6 Hz, H-1 Glc^{V}), 5.13 (d, 1H, J=3.6 Hz, H-1 Glc^{I}), 4.69 (d, 1H, J=7.9 Hz, H-1 Glc^{IV}).

ESI-MS, negative mode, m/z: 991.8 [M+3DBA−5H]²⁻, 927.1 [M+2DBA−4H]²⁻.

### Example 44

This example was prepared according to Method O (yield: 88%).

¹H NMR (400 MHz, D₂O, ppm), δ: 5.41-5.33 (m, 2H, H-1), 5.31 (d, 1H, J=2.6 Hz, H-1), 4.99 (d, 1H, H-1), 4.56 (d, 1H, J=8.0 Hz, H-1).

ESI-MS, negative mode, m/z: 1117.9 [M+4DBA−6H]²⁻, 1053.3 [M+3DBA−5H]²⁻, 988.7 [M+2DBA−4H]²⁻.

### Example 45

This example was prepared according to Method O (yield: 79%).

¹H NMR (400 MHz, D₂O, ppm), δ: 5.46 (d, 1H, J=3.5 Hz, H-1 Glc^{III}), 5.44 (s, 1H, H-1 ManUA^{II}), 5.39 (d, 1H, J=3.7 Hz, H-1 Glc^{V}), 5.09 (d, 1H, J=3.6 Hz, H-1 Glc^{I}), 4.65 (d, 1H, J=8.4 Hz, H-1 Glc^{IV}).

ESI-MS, negative mode, m/z: 1125.4 [M+4DBA−6H]²⁻, 1060.3 [M+3DBA−5H]²⁻.

### Example 46

This example was prepared according to Method P (yield: 88%).

¹H NMR (400 MHz, D₂O, ppm), δ: 5.48-5.41 (m, 3H, H-1), 5.39 (d, 1H, J=3.6 Hz, H-1), 5.09 (d, 1H, J=3.6 Hz, H-1).

ESI-MS, negative mode, 772/z: 1126.9 [M+4DBA−6H]²⁻, 1061.8 [M+3DBA−5H]²⁻, 621.4 [M+DBA−4H]³⁻, 578.3 [M−3H]³⁻.

### Example 47

This example was prepared according to Method Q (yield: 99%).

¹H NMR (400 MHz, D₂O, ppm), δ: 5.46 (d, 1H, J=3.5 Hz, H-1 Glc^{III}), 5.40 (d, 1H, J=3.7 Hz, H-1 Glc^{V}), 5.37 (s, 1H, H-1 ManUA^{II}), 5.02 (d, 1H, J=3.5 Hz, H-1 Glc^{I}), 4.65 (d, 1H, J=7.9 Hz, H-1 Glc^{IV}).

ESI-MS, negative mode, m/z: 995.8 [M+3DBA−5H]²⁻, 931.2 [M+2DBA−4H]²⁻.

### Example 48

To a solution of arachidonic acid (3.2 mg, 2 eq./pentasaccharide) in anhydrous DMF (0.380 ml) wad added TBTU (1-[bis(dimethylamino)methylene]-1H-benzotriazolium tetrafluoroborate 3-oxide, 3.4 mg, 2 eq./pentasaccharide) and diisopropylamine (53 μl, 2 eq./pentasaccharide). The mixture was stirred at room temperature for 1 h 45. This solution was then added in a solution of pentasaccharide (1 molar equivalent) in anhydrous DMF (0.2 ml). The mixture was stirred at room temperature for 19 h. After this time, 10 drops of a saturated aqueous solution of NaHCO₃ was added. The mixture was stirred at room temperature for 1 h. It was then filtered and the solution was directly poured onto a Sephadex LH-20 column (370 mL) equilibrated with DMF, to give the acylated pentasaccharide.

¹H NMR (400 MHz, D₂O, ppm), δ: 5.40 (d, 1H, J=3.8 Hz, H-1), 5.08 (d, 1H, J=3.8 Hz, H-1), 4.65 (d, 1H, J=8.1 Hz, H-1 Glc^{IV}).

ESI-MS, negative mode, m/z: 1072.8 [M+3DBA−5H]²⁻, 1008.2 [M+2DBA−4H]²⁻, 585.6 [M−3H]³⁻.

### Example 49

This example was prepared according to Method O (yield: 96%).

¹H NMR (400 MHz, D₂O, ppm), δ: 5.49 (s, 1H, H-1), 5.46 (d, 1H, J=3.8 Hz, H-1), 5.40 (d, 1H, J=3.7 Hz, H-1), 5.08 (d, 1H, J=3.6 Hz, H-1), 4.65 (d, 1H, H-1).

ESI-MS, negative mode, m/z: 1061.8 [M+3DBA−5H]²⁻, 621.4 [M+DBA−4H]³⁻, 578.3 [M−3H]³⁻.

### Example 50

This example was prepared according to Method O (yield: 66%).

¹H NMR (400 MHz, D₂O, ppm), δ: 5.53-5.45 (m, 2H, H-1), 5.43 (d, 1H, J=3.4 Hz, H-1 Glc^{V}), 5.16-5.10 (m, 1H, H-1), 4.69 (d, 1H, J=8.0 Hz, H-1 Glc^{IV}).

ESI-MS, negative mode, m/z: 995.2 [M+3DBA−5H]²⁻, 930.6 [M+2DBA−4H]²⁻.

### Example 51

This example was prepared according to Method H (yield: 70%).

¹H NMR (400 MHz, D₂O, ppm), δ: 5.24 (d, 1H, J=3.6 Hz, H-1 Glc^{III}), 5.18 (s, 1H, H-1), 5.11 (d, 1H, J=3.5 Hz, H-1 Glc^{V}), 4.91 (d, 1H, J=3.5 Hz, H-1 Glc^{I}), 4.42 (d, 1H, J=7.9 Hz, H-1 Glc^{IV}).

ESI-MS, negative mode, m/z: 838.0 [M+2DBA−4H]²⁻, 773.4 [M+DBA−3H]²⁻.

### Example 52

This example was prepared according to preparation 14 (compound 162).

### Example 53

This example was prepared according to Method O (yield: 78%).

¹H NMR (400 MHz, D₂O, ppm), δ: 5.53-5.47 (m, 2H, H-1 ManUA^{II}, H-1 Glc^{III}), 5.38 (d, 1H, J=3.8 Hz, H-1 Glc^{V}), 5.18 (d, 1H, J=3.5 Hz, H-1 Glc^{I}), 4.59 (d, 1H, J=8.0 Hz, H-1 Glc^{IV}).

ESI-MS, negative mode, m/z: 1174.0 [M+3DBA−5H]²⁻, 695.8 [M+DBA−4H]³⁻.

### Example 54

This example was prepared according to Method O (yield: 76%).

¹H NMR (400 MHz, D₂O, ppm), δ: 5.50-5.45 (m, 2H, H-1), 5.38 (d, 1H, J=3.8 Hz, H-1), 5.16 (d, 1H, J=3.5 Hz, H-1).

ESI-MS, negative mode, m/z: 1074.3 [M+3DBA−5H]²⁻, 1009.7 [M+2DBA−4H]²⁻, 629.7 [M+DBA−4H]³⁻.

### Example 55

This example was prepared according to Method Q (yield: 60%).

¹H NMR (400 MHz, D₂O, ppm), δ: 5.52-5.44 (m, 2H, H-1), 5.40 (d, 1H, J=3.6 Hz, H-1), 5.16 (d, 1H, J=3.4 Hz, H-1 Glc^{I}), 4.63 (d, 1H, J=7.6 Hz, H-1 Glc^{IV}).

ESI-MS, negative mode, m/z: 1049.7 [M+3DBA−5H]²⁻, 613.3 [M+DBA−4H]³⁻.

### Example 56

This example was prepared according to Method Q (yield: 80%).

¹H NMR (400 MHz, D₂O, ppm), δ: 5.48 (s, 1H, H-1 ManUA^{II}), 5.43 (d, 1H, J=3.8 Hz, H-1 Glc^{I}), 5.40 (d, 1H, J=3.8 Hz, H-1 Glc^{V}), 5.17 (d, 1H, J=3.5 Hz, H-1 Glc^{III}), 4.50 (d, 1H, J=7.5 Hz, H-1 Glc^{IV}).

ESI-MS, negative mode, m/z: 995.7 [M+3DBA−5H]²⁻, 931.1 [M+2DBA−4H]²⁻.

### Example 57

This example was: prepared according to Method O (yield: 68%).

¹H NMR (400 MHz, D₂O, ppm), δ: 5.41-5.37 (m, 2H, H-1), 5.29 (d, 1H, J=3.7 Hz, H-1), 5.08 (d, 1H, J=3.2 Hz, H-1), 5.03 (s, 1H, H-1).

ESI-MS, negative mode, m/z: 1053.2 [M+3DBA−5H]²⁻, 988.6 [M+2DBA−4H]²⁻, 615.7 [M+DBA−4H]³⁻.

### Example 58

This example was prepared according to Method Q (yield: 67%).

¹H NMR (400 MHz, D₂O, ppm), δ: 5.47-5.43 (m, 2H, H-1), 5.37 (d, 1H, J=3.8 Hz, H-1), 5.13 (d, 1H, J=3.7 Hz, H-1).

ESI-MS, negative mode, m/z: 991.7 [M+3DBA−5H]²⁻, 574.7 [M+DBA−4H]³⁻.

### Example 59

This example was prepared according to Method P (yield: 45%).

¹H NMR (400 MHz, D₂O, ppm), δ: 5.47-5.43 (m, 2H, H-1), 5.37 (d, 1H, J=3.5 Hz, H-1), 5.13 (d, 1H, J=3.2 Hz, H-1).

ESI-MS, negative mode, m/z: 978.7 [M+3DBA−5H]²⁻, 914.1 [M+2DBA−4H]²⁻, 849.5 [M+DBA−3H]²⁻.

### Example 60

This example was prepared according to Method T (yield: 96%).

¹H NMR (400 MHz, D₂O, ppm), δ: 5.51 (s, 1H, H-1 ManUA^{II}), 5.49 (d, 1H, J=3.4 Hz, H-1 Glc^{III}), 5.40 (d, 1H, J=3.4 Hz, H-1 Glc^{V}), 5.17 (d, 1H, J=3.4 Hz, H-1 Glc^{I}), 4.60 (d, 1H, J=8.1 Hz, H-1 Glc^{IV}).

ESI-MS, negative mode, m/z: 921.6 [M+2DBA−4H]²⁻.

O-Alkyl/NHR family: R₁₄, R₁₅═O-Alkyl/O-Arylalkyl, R₁₃: NHR″
Compounds Derived from 4S Templates

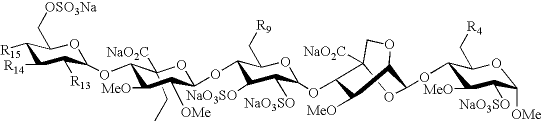

| Example | R₁₄/R₁₅ | R₁₃ | R₉ | R₄ |
|---|---|---|---|---|
| 61 | OBn | N₃ | OH | OH |
| 62 | OBu | N₃ | OH | OH |
| 63 | OMe | N₃ | OH | OH |
| 64 | OHex | N₃ | OH | OH |
| 65 | OBu | NH₂ | OH | OH |
| 66 | OBu | NHDOCA | OH | OH |
| 67 | OBu | NH(Z-amino) | OH | OH |
| | | hexanoyl | | |
| 68 | OBu | NHSNAD | OH | OH |
| 69 | OBu | NHoleyl | OH | OH |
| 70 | OBu | NH(3-cyclopentylpropanoyl) | OH | OH |
| 71 | OBu | NHhydrocinnamoyl | OH | OH |
| 72 | OPhPr | N₃ | OH | OH |
| 73 | OBu | N₃ | OSO₃Na | N3 |
| 74 | OBu | NHDOCA | OSO₃Na | NHDOCA |
| 75 | OBu | NH(3,5- | OSO₃Na | NH(3,5- |
| | | bis(trifluoromethyl) | | bis(trifluoromethyl) |
| | | benzoyl) | | benzoyl) |
| 76 | OBu | NHhydrocinnamoyl | OSO₃Na | NHhydrocinnamoyl |
| 77 | OBu | NHDOCA | NHDOCA | OSO₃Na |
| 78 | OBu | N₃ | N₃ | OSO₃Na |
| 79 | OBu | NHSNAD | NHSNAD | OSO₃Na |
| 80 | OBu | NH(3,5- | NH(3,5- | OSO₃Na |
| | | bis(trifluoromethyl) | bis(trifluoromethyl) | |
| | | benzoyl) | benzoyl) | |
| 81 | OBu | NH(Z-amino)hexanoyl | NH(Z-amino)hexanoyl | OSO₃Na |
| 82 | OBn | N₃ | OSO₃Na | OSO₃Na |
| 83 | OBu | N₃ | OSO₃Na | OSO₃Na |
| 84 | OHex | N₃ | OSO₃Na | OSO₃Na |
| 85 | OMe | N₃ | OSO₃Na | OSO₃Na |
| 86 | OBu | N(CH₃)₂ | OSO₃Na | OSO₃Na |
| 87 | OHex | NH₂ | OSO₃Na | OSO₃Na |
| 88 | OHex | NHDOCA | OSO₃Na | OSO₃Na |

### Example 61

This example was prepared according to preparation 14 (compound 150).

### Example 62

This example was prepared according to preparation 14 (compound 151).

### Example 63

This example was prepared according to preparation 14 (compound 152).

### Example 64

This example was prepared according to preparation 14 (compound 153).

### Example 65

This example was prepared according to Method T (yield=84%).

¹H NMR (400 MHz, D₂O, ppm), δ: 5.42 (d, 1H, J=3.7 Hz, H-1 Glc^{III}), 5.06 (d, 1H, J=7.6 Hz, H-1 Glc^{IV}), 5.02 (s, 1H, H-1 ManUA^{II}), 4.92 (d, 1H, J=3.5 Hz, H-1), 4.84 (d, 1H, J=3.3 Hz, H-1).

ESI-MS, negative mode, m/z: 697.9 [M−2H]²⁻, 464.9 [M−3H]³⁻.

### Example 66

This example was prepared according to Method O (yield=74%).

¹H NMR (400 MHz, CD₃OD, ppm), δ: 5.51 (d, 1H, J=3.6 Hz, H-1 Glc^{III}), 5.16 (d, 1H, J=8.1 Hz, H-1 Glc^{IV}), 5.10 (s, 1H, H-1 ManUA^{II}), 5.01 (d, 1H, J=3.6 Hz, H-1 Glc^{I}), 4.91 (d, 1H, J=3.6 Hz, H-1 Glc^{V})

ESI-MS, negative mode, m/z: 1071.4 [M+2DBA−4H]²⁻, 1006.3 [M+DBA−3H]²⁻.

### Example 67

This example was prepared according to Method O (yield=84%).

¹H NMR (400 MHz, CD₃OD, ppm), δ: 5.42 (d, 1H, J=3.5 Hz, H-1 Glc^{III}), 5.08 (d, 1H, J=7.6 Hz, H-1 Glc^{IV}), 5.02 (s, 1H, H-1 ManUA^{II}), 4.93 (d, 1H, J=3.0 Hz, H-1), 4.82 (d, 1H, J=3.1 Hz, H-1).

ESI-MS, negative mode, m/z: 950.8 [M+2DBA−4H]²⁻, 886.2 [M+DBA−3H]²⁻.

### Example 68

This example was prepared according to Method O (yield=77%).

¹H NMR (400 MHz, CD₃OD, ppm), δ: 5.42 (d, 1H, J=3.8 Hz, H-1), 5.06 (d, 1H, J=7.7 Hz, H-1), 5.02 (s, 1H, H-1), 4.93 (d, 1H, J=3.3 Hz, H-1).

ESI-MS, negative mode, m/z: 971.8 [M+2DBA−4H]²⁻, 907.2 [M+DBA−3H]²⁻, 561.4 [M−3H]³⁻.

### Example 69

This example was prepared according to Method P (yield=95%).

¹H NMR (400 MHz, CD₃OD, ppm), δ: 5.42 (d, 1H, J=3.4 Hz, H-1), 5.06 (d, 1H, J=8.1 Hz, H-1), 5.02 (s, 1H, H-1), 4.94 (d, 1H, J=3.4 Hz, H-1), 4.83 (d, 1H, J=3.4 Hz, H-1).

ESI-MS, negative mode, m/z: 959.4 [M+2DBA−4H]²⁻, 894.8 [M+DBA−3H]²⁻, 553.1 [M−3H]³⁻.

### Example 70

This example was prepared according to Method Q (yield: 77%).

¹H NMR (400 MHz, D₂O, ppm), δ: 5.49 (d, 1H, J=3.6 Hz, H-1), 5.46 (d, 1H, J=3.8 Hz, H-1), 5.25 (s, 1H, H-1 ManUA^{II}), 4.80 (d, 1H, J=7.8 Hz, H-1 Glc^{IV}), 3.06 (d, 1H, J=3.5 Hz, H-1).

ESI-MS, negative mode, m/z: 889.2 [M+2DBA−4H]²⁻, 824.6 [M+DBA−3H]²⁻.

### Example 71

This example was prepared according to Method Q (yield: 71%).

¹H NMR (400 MHz, D₂O, ppm), δ: 5.41 (d, 1H, J=3.8 Hz, H-1), 5.05-4.99 (m, 2H, H-1), 4.92 (d, 1H, J=3.5 Hz, H-1), 4.80 (d, 1H, J=3.7 Hz, H-1).

ESI-MS, negative mode, m/z: 829.6 [M+DBA−3H]²⁻.

### Example 72

This example was prepared according to preparation 14 (compound 158).

### Example 73

This example was prepared according to preparation 14 (compound 160).

### Example 74

This example was prepared according to Method O (yield: 77%).

¹H NMR (400 MHz, CD₃OD, ppm), δ: 5.44 (d, 1H, J=3.3 Hz, H-1), 4.96-4.86 (m, 3H, H-1).

ESI-MS, negative mode, m/z: 859.8 [M+DBA−4H]³⁻, 816.7 [M−3H]³⁻.

### Example 75

This example was prepared according to Method Q (yield: 92%).

¹H NMR (400 MHz, CD₃OD, ppm), δ: 5.47 (d, 1H, J=3.6 Hz, H-1), 5.08-5.03 (m, 2H, H-1), 4.93 (d, 1H, J=3.7 Hz, H-1), 4.87 (d, 1H, J=7.4 Hz, H-1).

ESI-MS, negative mode, m/z: 694.4 [M+DBA−4H]³⁻, 651.4 [M−3H]³⁻.

### Example 76

This example was prepared according to Method Q (yield: 88%)

¹H NMR (400 MHz, CD₃OD, ppm), δ: 5.46 (d, 1H, J=3.5 Hz, H-1), 4.99-4.86 (m, 4H, H-1).

ESI-MS, negative mode, m/z: 1063.9 [M+3DBA−5H]²⁻, 998.8 [M+2DBA−4H]²⁻.

### Example 77

This example was prepared according to Method O (yield=87%).

¹H NMR (400 MHz, CD₃OD, ppm), δ: 5.40 (d, 1H, J=3.4 Hz, H-1), 5.13 (s, 1H, H-1 ManUA^{II}), 5.01 (d, 1H, J=8.0 Hz, H-1 Glc^{IV}), 4.93-4.88 (m, 2H, H-1).

ESI-MS, negative mode, m/z: 859.8 [M+2DBA−5H]³⁻, 816.7 [M−3H]³⁻.

### Example 78

This example was prepared according to preparation 14 (compound 157).

### Example 79

This example was prepared according to Method O (yield: 86%).

¹H NMR (400 MHz, CD₃OD, ppm), δ: 5.45 (d, 1H, J=3.4 Hz, H-1), 5.11 (s, 1H, H-1), 4.91 (d, 1H, J=3.4 Hz, H-1).

ESI-MS, negative mode, m/z: 1156.4 [M+2DBA−4H]²⁻, 727.2 [M+DBA−4H]³⁻.

### Example 80

This example was prepared according to Method Q (yield: 97%).

¹H NMR (400 MHz, CD₃OD, ppm), δ: 5.37 (d, 1H, J=3.1 Hz, H-1), 5.11-5.04 (m, 2H, H-1), 4.93-4.85 (m, 2H, H-1).

ESI-MS, negative mode, m/z: 1106.7 [M+2DBA−4H]²⁻.

### Example 81

This example was prepared according to Method O (yield: 88%).

¹H NMR (400 MHz, D₂O, ppm), δ: 5.50 (d, 1H, J=3.3 Hz, H-1), 5.26 (s, 1H, H-1), 5.09-5.00 (m, 2H, H-1), 4.97 (d, 1H, J=7.7 Hz, H-1).

ESI-MS, negative mode, m/z: 1114.3 [M+2DBA−4H]²⁻, 699.4 [M+DBA−4H]³⁻, 656.0 [M−3H]³⁻.

### Example 82

This example was prepared according to Method S (yield: 80%).

¹H NMR (400 MHz, D₂O, ppm), δ: 5.46-5.37 (m, 2H, H-1), 5.10 (s, 1H, H-1), 4.91 (d, 1H, J=3.0 Hz, H-1), 4.68 (1H, H-1).

ESI-MS, negative mode, m/z: 1083.3 [M+4DBA−6H]²⁻, 1018.7 [M+3DBA−5H]²⁻, 954.1 [M+2DBA−4H]²⁻, 592.6 [M+DBA−4H]³⁻.

### Example 83

This example was prepared according to Method S (yield: 94%).

¹H NMR (400 MHz, CD₃OD, ppm), δ: 5.45 (d, 1H, J=3.3 Hz, H-1 Glc^{III}), 5.37 (d, 1H, J=3.7 Hz, H-1 Glc^{V}), 5.10 (s, 1H, H-1 ManUA^{II}), 4.92 (d, 1H, J=3.5 Hz, H-1 Glc^{I}), 4.70 (d, 1H, J=7.9 Hz, H-1 Glc^{IV}).

ESI-MS, negative mode, m/z: 984.7 [M+3DBA−5H]²⁻, 920.1 [M+2DBA−4H]²⁻.

### Example 84

This example was prepared according to Method S (yield: 95%).

¹H NMR (400 MHz, CD₃OD, ppm), δ: 5.41 (d, 1H, J=2.9 Hz, H-1 Glc^{III}), 5.33 (d, 1H, J=3.7 Hz, H-1), 5.07 (s, 1H, H-1 ManUA^{II}), 4.89 (d, 1H, J=3.4 Hz, H-1), 4.67 (d, 1H, H-1).

ESI-MS, negative mode, m/z: 1077.3 [M+4DBA−6H]²⁻, 1012.8 [M+3DBA−5H]²⁻, 948.2 [M+2DBA−4H]²⁻.

### Example 85

This example was prepared according to Method S (yield: 97%)

¹H NMR (400 MHz, CD₃OD, ppm), δ: 5.43 (d, 1H, J=3.7 Hz, H-1 Glc^{III}), 5.36 (d, 1H, J=3.5 Hz, H-1 Glc^{V}), 5.22 (s, 1H, H-1 ManUA^{II}), 5.01 (d, 1H, J=3.6 Hz, H-1 Glc^{I}), 4.62 (d, 1H, J=8.0 Hz, H-1 Glc^{IV}).

ESI-MS, negative mode, m/z: 943.1 [M+3DBA−5H]²⁻, 878.5 [M+2DBA−4H]²⁻, 541.9 [M+DBA−4H]³⁻.

### Example 86

A solution of pentasaccharide (7.4 mg, 4.3 mmol) in methanol (0.74 mL) was stirred under hydrogen in the presence of Pd10%/C catalyst (3.7 mg) and formaldehyde 37% (48 μl, 150 eq) for 48 h and filtered through PTI-E, millipore membrane. The solution was concentrated to give the hydrogenolysed pentasaccharide (7.0 mg, yield: 93%).

¹H NMR (400 MHz, CD₃OD, ppm), δ: 5.45 (d, 1H, J=2.9 Hz, H-1 Glc^{III}), 3.43 (d, 1H, J=3.5 Hz, H-1 Glc^{V}), 3.09 (s, 1H, H-1 ManUA^{II}), 4.90 (d, 1H, J=3.6 Hz, H-1 Glc^{I}), 4.85 (d, 1H, J=7.5 Hz, H-1 Glc^{IV}).

ESI-MS, negative mode, m/z: 921.1 [M+2DBA−4H]²⁻.

### Example 87

This example was prepared according to Method T (yield: 99%).

ESI-MS, negative mode, m/z: 935.2 [M+2DBA−4H]²⁻, 870.6 [M+DBA−3H]²⁻.

### Example 88

This example was prepared according to Method O (yield: 67%).

¹H NMR (400 MHz, CD₃OD, ppm), δ: 5.44 (d, 1H, J=3.4 Hz, H-1 Glc^{I}), 5.09 (s, 1H, H-1 ManUA^{II}), 4.95-4.88 (m, 3H, H-1 Glc^{III}, H-1 Glc^{IV}, H-1 Glc^{V}).

ESI-MS, negative mode, m/z: 786.0 [M+2DBA−5H]³⁻, 742.9 [M−1-DBA−4H]³⁻.

Compounds Derived from 5S Templates

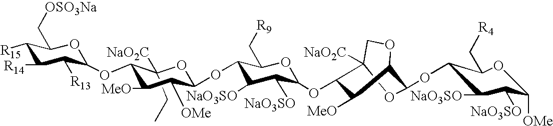

| Example | R₁₄/R₁₅ | R₁₃ | R₉ | R₄ |
|---|---|---|---|---|
| 89 | OBn | N₃ | OSO₃Na | OSO₃Na |
| 90 | OH | NHdecanoyl | OSO₃Na | OSO₃Na |
| 91 | OH | NH₂ | OSO₃Na | OSO₃Na |
| 92 | OH | NHhexanoyl | OSO₃Na | OSO₃Na |

### Example 89

This example was prepared according to Method S (yield: 78%).

¹H NMR (400 MHz, D₂O, ppm), δ: 5.41 (d, 1H, J=3.2 Hz, H-1 Glc^{III}), 5.38 (s, 1H, H-1 ManUA^{II}), 5.33 (d, 1H, J=3.5 Hz, H-1 Glc^{V}), 5.04 (d, 1H, J=3.2 Hz, H-1 Glc^{I}).

ESI-MS, negative mode, m/z: 1063.7 [M+4DBA−6H]²⁻, 999.1 [M+3DBA−5H]²⁻, 934.4 [M+2DBA−4H]²⁻.

### Example 90

This example was prepared according to Method P (yield: 59%).

¹H NMR (400 MHz, D₂O, ppm), δ: 5.50-5.38 (m, 2H, H-1), 5.14-5.04 (m, 3H, H-1).

ESI-MS, negative mode, m/z: 1091.7 [M+4DBA−6H]²⁻, 1027.1 [M+3DBA−5H]²⁻, 962.5 [M+2DBA−4H]²⁻.

### Example 91

This example was prepared according to Method T (yield: 99%).

¹H NMR (400 MHz, D₂O, ppm), δ: 5.55-5.44 (m, 2H, H-1 ManUA^{II}, H-1 Glc^{III}), 5.31 (d, 1H, J=3.3 Hz, H-1 Glc^{V}), 5.17 (d, 1H, J=3.5 Hz, H-1 Glc^{I}), 4.92 (d, 1H, J=7.25 Hz, H-1 Glc^{IV}).

ESI-MS, negative mode, m/z: 949.0 [M+3DBA−5H]²⁻, 884.4 [M+2DBA−4H]²⁻.

### Example 92

This example was prepared according to Method P (yield: 57%).

¹H NMR (400 MHz, D₂O, ppm), δ: 5.57-5.50 (m, 2H, H-1), 5.21 (d, 1H, J=3.4 Hz, H-1), 5.18 (d, 1H, J=3.5 Hz, H-1), 5.03 (d, 1H, J=7.7 Hz, H-1 Glc^{IV}).

ESI-MS, negative mode, m/z: 1063.7 [M+4DBA−6H]²⁻, 999.1 [M+3DBA−5H]²⁻, 934.4 [M+2DBA−4H]²⁻.

### Example 93

### Synthesis of Methyl O-(2,3,4-tri-O-butyl-6-O-sulfo-α-D-glucopyranosyl)-(1→4)-O-(2-O-butyl-5-C-ethyl-3-O-methyl-β-D-glucopyranosyluronic acid)-(1→4)-O-(6-O-butyl-2,3-di-O-sulfo-α-D-glucopyranosyl)-(1→4)-O-(2,6-anhydro-5-C-carboxy-3-O-methyl-β-D-mannopyranosyl)-(1→4)-3,6-di-O-butyl-2-O-sulfo-α-D-glucopyranoside, hexasodium salt 161

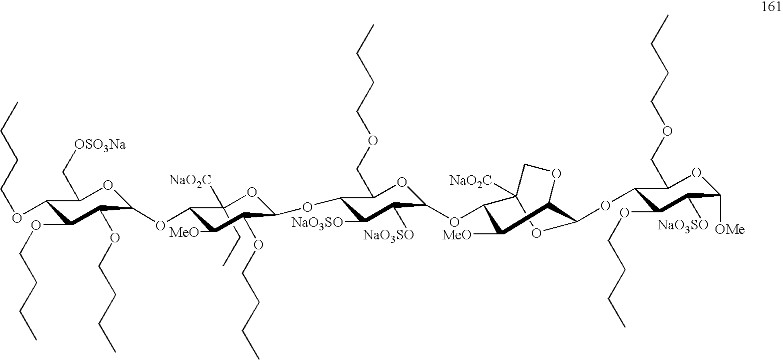

Pentasaccharide 38 (10 mg, 7.2 μmol) was alkylated with 1-bromobutane according to ‘Method C: Alkylation’ to give pentasaccharide 161 (10.4 mg, 81%), which had the following properties: chemical shifts of the anomeric protons: 5.42, 5.38, 5.15, 4.74 and 4.68 ppm; and MS (ESI⁻): chemical mass=1782.47; experimental mass=1783.4.

### Example 94

### Synthesis of Methyl O-(2,3,4-tri-O-nonanoyl-6-O-sulfo-α-D-glucopyranosyl)-(1→4)-O-(5-C-ethyl-3-O-methyl-2-O-nonanoyl-β-D-glucopyranosyluronic nonanoyl-2,3-di-O-sulfo-α-D-glucopyranosyl)-(1→4)-O-(2,6-anhydro-5-C-carboxy-3-O-methyl-β-D-mannopyranosyl)-(1→4)-3,6-di-O-nonanoyl-2-O-sulfo-α-D-glucopyranoside, hexasodium salt 162

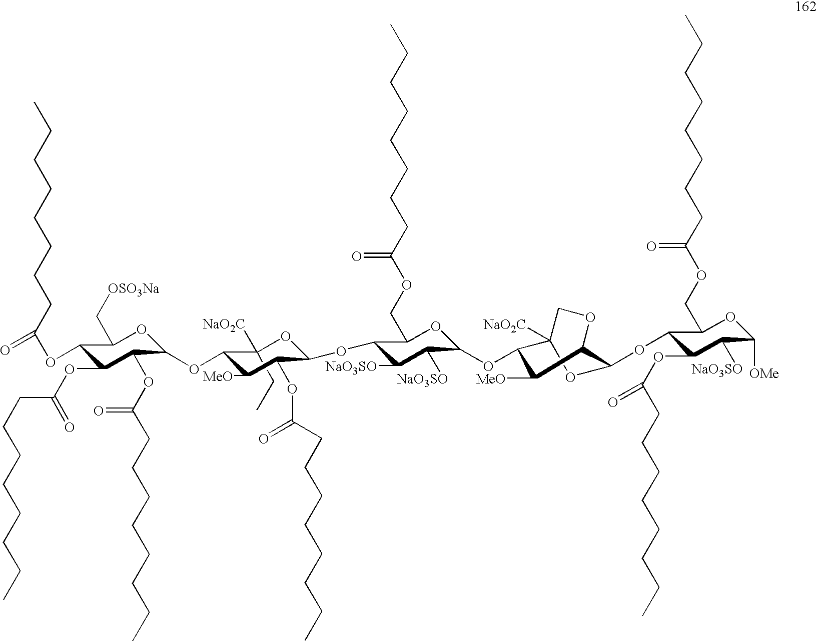

Pentasaccharide 38 (10 mg, 7.2 μmol) was acylated with 2-nonanoyl chloride according to ‘Method D: Acylation’ to give pentasaccharide 162 (13.4 mg, 78%), which had the following properties: chemical shifts of the anomeric protons: 5.54, 5.52, 5.21, 4.86 and 4.72 ppm; and MS (ESI⁻): chemical mass=2370.87; experimental mass=2372.1.

### Example 95

### Synthesis of Methyl O-(6-O-sulfo-2,3,4-tri-O-(4-tert-butylbenzyl)-α-D-glucopyranosyl)-(1→4)-O-(5-C-ethyl-3-O-methyl-2-O-(4-tert-butylbenzyl)-β-D-glucopyranosyluronic acid)-(1→4)-O-(6-O-cyclopentanepropionyl-2,3-di-O-sulfo-α-D-glucopyranosyl)-(1→)-O-(2,6-anhydro-5-C-carboxy-3-O-methyl-β-D-mannopyranosyl)-(1→4)-6-O-cyclopentane propionyl-2-O-sulfo-3-O-(4-tert-butylbenzyl)-α-D-glucopyranoside, hexasodium salt 163

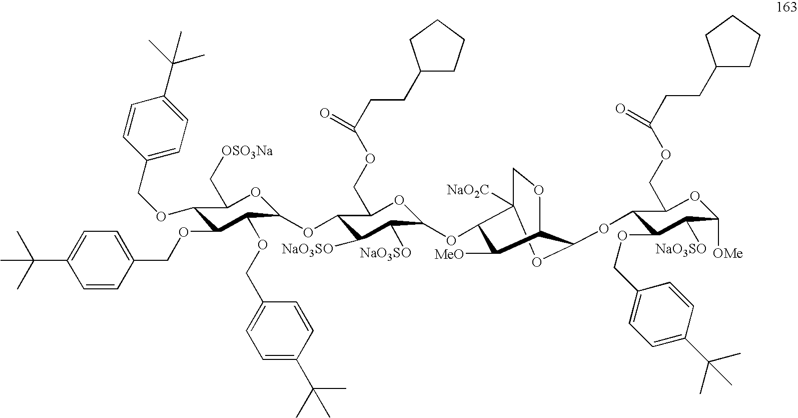

Pentasaccharide 37 (19 mg, 10.1 μmol) was alkylated with 4-tert-butylbenzyl chloride according to ‘Method C: Alkylation’. The resulting compound was desilylated in the manner described in ‘Method A: Desilylation’, and acylated with cyclopentanepropionyl chloride according to ‘Method D: Acylation’ to give pentasaccharide 163 (12.7 mg, 81%), which had the following properties: chemical shifts of the anomeric protons: 5.52, 5.48, 5.26, 4.89 and 4.68 ppm; and MS (ESI⁻): chemical mass=2230.82; experimental mass=2231.9.

### Example 96

### Synthesis of Methyl O-(2,3,4-tri-O-hexanoyl-6-O-sulfo-α-D-glucopyranosyl)-(1→4)-O-(5-C-ethyl-2-O-hexanoyl-3-O-methyl-β-D-glucopyranosyluronic acid-(1→4)-O-(6-O-(2,2-dimethylpropyl)-2,3-di-O-sulfo-α-D-glucopyranosyl)-(1→4)-O-(2,6-anhydro-5-C-carboxy-3-O-methyl-β-D-mannopyranosyl)-(1→4)-6-O-(2,2-dimethylpropyl)-3-O-hexanoyl-2-O-sulfo-α-D-glucopyranoside, hexasodium salt 164

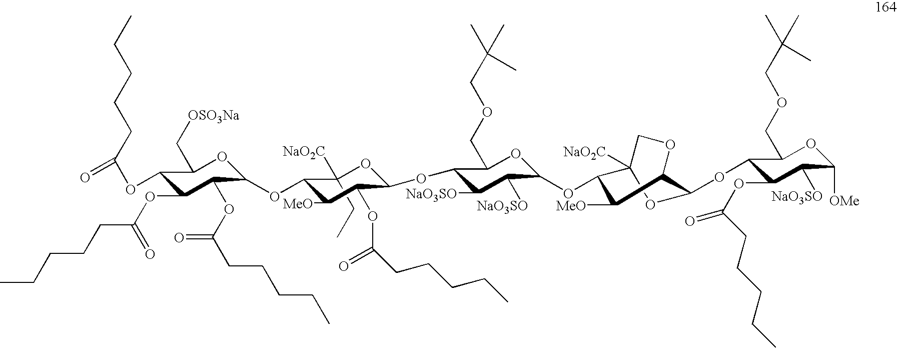

Pentasaccharide 36 (18 mg, 9.1 μmol) was alkylated with 1-bromo-2,2-dimethylpropane according to ‘Method C: Alkylation’. The resulting compound was hydrogenolysed in a manner as described in ‘Method B: Hydrogenolysis’, and acylated with hexanoyl chloride according to ‘Method D: Acylation’ to give pentasaccharide 164 (12.3 mg, 67%), which had the following properties: chemical shifts of the anomeric protons: 5.56, 5.49, 5.26, 4.89 and 4.71 ppm; and MS (ESI⁻): chemical mass=1882.61; experimental mass=1883.7.

### Example 97

### Synthesis of Methyl O-(2,3,4-tri-O-(4-chlorobenzyl)-6-O-sulfo-α-D-glucopyranosyl)-(1→4)-O-(2-O-(4-chlorobenzyl)-5-C-ethyl-3-O-methyl-β-D-glucopyranosyluronic acid)-(1→4)-O-(2,3-di-O-sulfo-α-D-glucopyranosyl)-(1→4)-O-(2,6-anhydro-5-C-carboxy-3-O-methyl-β-D-mannopyranosyl)-(1→4)-3-O-(4-chlorobenzyl)-2-O-sulfo-α-D-glucopyranoside, hexasodium salt 165

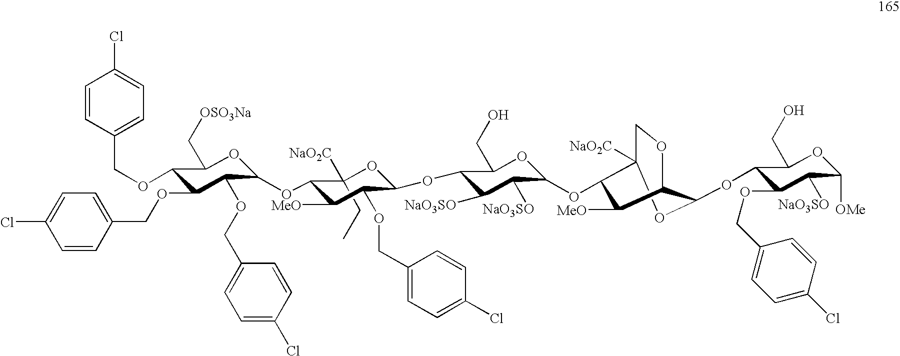

Pentasaccharide 37 (16 mg, 8.6 μmmol) was alkylated with 4-chlorobenzyl chloride according to ‘Method C: Alkylation’. The resulting compound was desilylated in the manner described in ‘Method A: Desilylation’ to give pentasaccharide 165 (12.4 mg, 72%), which had the following properties: chemical shifts of the anomeric protons: 5.67, 5.62, 5.22, 4.89 and 4.64 ppm; and MS (ESI⁻): chemical mass=2010.07; experimental mass=1911.3.

### Example 98

### Synthesis of Methyl O-(6-O-sulfo-α-D-glucopyranosyl)-(1→4)-O-(5-C-ethyl-3-O-methyl-β-D-glucopyranosyluronic acid)-(1→4)-O-(6-O-(deoxycholoyl)-2,3-di-O-sulfo-α-D-glucopyranosyl)-(1→4)-O-(2,6-anhydro-5-C-carboxy-3-O-methyl-β-D-mannopyranosyl)-(1→4)-6-O-(deoxycholoyl)-2-O-sulfo-α-D-glucopyranoside, disodium and tetra-tripropyl ammonium salt 166

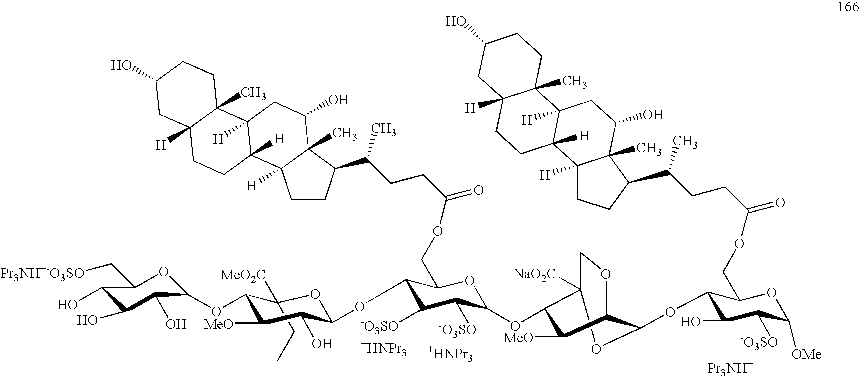

Pentasaccharide 36 (18 mg, 9.1 μmol) was acylated with deoxycholoyl chloride according to ‘Method D: Acylation’. The resulting compound was hydrogenolysed in the manner described in ‘Method B: Hydrogenolysis’. The resulting pentasaccharide sodium salt was dissolved in water and an aqueous solution of tripropyl ammonium chloride (4 equivalents) was added. The mixture was stirred at room temperature for 16 h. The solution was then loaded on top of a Sephadex G25F column (50 mL) equilibrated with water. The fractions containing the compound were collected and concentrated to give the pentasaccharide-tripropyl ammonium ion complex 166 (10.7 mg, 45%), which had the following properties: chemical shifts of the anomeric protons: 5.68, 5.62, 5.22, 4.89 and 4.64 ppm; and MS (ESI⁻): chemical mass=2000.66; experimental mass=2001.7.

### Biological Testing

It will be understood that a variety of assays are suitable for testing the biological activity of the compounds of the present invention. However, suitable methods for testing the biological activity of the compounds of the present invention are listed below.

### Determination of Anti-Factor Xa Activity of Compounds

IC₅₀ values of compounds were determined by their anti-factor Xa activity using a Stachrom HP kit (Diagnostica Stago). This assay was carried out on a STA Compact (Diagnostica Stago).

The anti-factor-Xa activity was determined by the same way that it has been for fondaparinux, which was used as standard (see below).

Fondaparinux+AT (excess)→[Fondaparinux·AT] 1.

[Fondaparinux·AT]+fXa (excess)→[Fondaparinux·AT·fXa]+fXa (remaining) 2.

Chromogenic substrate→Peptide+pNA 3.

Fondaparinux was analysed as a complex with Antithrombin (AT) present in the sample. The concentration of this complex was dependent on availability of AT. In order to obtain a more constant concentration of AT, purified AT was added to the test plasma. factor Xa (in excess) was neutralized in proportion to the amount of fondaparinux, which determine the amount of [Fondaparinux·AT] complex. The remaining amount of fXa hydrolyzed the chromogenic substrate thus liberating the chromophoric group, pNA. The colour was then read photometrically at 405 nm.

### Quantification of Compounds in Plasma

Rat plasmatic concentration of compounds (μg compound/mL plasma) was determined by their anti-factor Xa activity using factor Xa activity using a Stachrom HP kit (Diagnostica Stago) as described above. This assay was carried out on a STA Compact (Diagnostica Stago). A specific standard curve was preformed with each compound which was quantified in rat plasma.

### Example—Quantification of the Compound in Rat Plasma and Pharmacokinetic Profile Determination for Oral and Intravenous Administration

Rat plasmatic concentration of compounds of the present invention was determined by anti factor Xa activity as described previously.

The compounds were prepared in solution ready for oral and intravenous administration, and the doses were varied. In human, oral administration is the preferred route administration.

The pharmacokinetics of the compounds of the present invention were investigated in female Wistar Han rats.

Rat blood (9 volumes) was mixed with sodium citrate (1 volume) and preferably cooled immediately on ice to minimize release of heparin antagonists from blood cells. As soon as possible after collection, the sample was subjected to a centrifugation at 3000×g for 10 minutes at low temperature (the plasma is typically stable for 24 h at temperature below 8° C.) and stored frozen at −20° C.

The Rat plasmatic concentration of compounds (μg compound/mL plasma) was determined by their anti-factor Xa activity using factor Xa activity as described above.

Pharmacokinetic Study of Compounds with Direct Intra Duodenal Injection:

Direct Intra Duodenal Injection (DIDI) has been used on the Wistar Han rats to estimate the ability of the compounds to cross the intestinal membrane. A laparatomy was performed on anesthetized rats in which the duodenum was exposed in order to inject a compound directly into the lumen of the intestine. This non survival surgical method allowed the compound to bypass the stomach.

Rats have been placed on their caudal side with their abdomen exposed and their head held downward to the facemask. The body temperature was maintained at 38° C. Fur was removed from approximately 150% larger that the area of the incision and loose fur should be carefully dusted away in order to prevent translocation into the incision. The intestine was exposed through a midline abdominal incision using a #20 blade and the upper small intestine i.e. the duodenum was isolated. A small pore was performed using a high temperature cautery fine tip unit 1-2 cm to the beginning of the duodenum and a flexible catheter was passed inside the hole into the duodenal lumen. After tubing with the flexible catheter, the duodenum was closed by clipping with a forceps. A syringe containing the drug solution (2 mg/kg BW) was placed onto the flexible catheter and the syringe's plunger was slowly depressed releasing the material into the duodenum. At this step, a two-layer closure in needed in which the body wall was closed separately from the skin using silk suture #4.0.

To collect blood into the tail vein, a disposable catheter was inserted by directing the needle into the vein. Blood was collected into citrate tubes (1 vol of citrate/9 vol of blood). The following general blood sampling schemes were commonly used in DIDI: 0′, 5′, 15′, 30′, 60′, 90′ and 120′). Plasmas were collected by centrifugation at 3500 rpm, 4° C., and stored frozen at =20° C.

The Rat plasmatic concentration of compounds (μg compound/mL plasma) was determined by their anti-factor Xa activity using factor Xa activity as described above.

### Gastro-Intestinal Stability

A gastric-intestinal stability assay has been performed in simulated fluids and the quantification has been performed with the anti-factor Xa assay as described above. The composition of the reconstituted fluid was comparable to the fluid that could be found in stomach and intestine of mammalians:
Simulated Gatric Fluid (SGF): NaCl 0.2%, HCl 0.7%, pepsin 0.32% in water, pH 1.2.
Simulated Intestinal Fluid (SIF): KH2PO4 0.68%, NaOH 0.2 M, Pancreatin 1% in water, pH7.5.

Study has been performed at 37° C. and samples were taken as a function of time every 30 min for a period of 3 h. The reaction was stopped by addition of 1M sodium bicarbonate to reach a pH of 7.2 for the SGF and by snap freezing at −20° C. for the SIR

### Results

### O-Alkyl/Family: R₁₃═R₁₄/R₁₅

Compounds Derived from 4S Templates

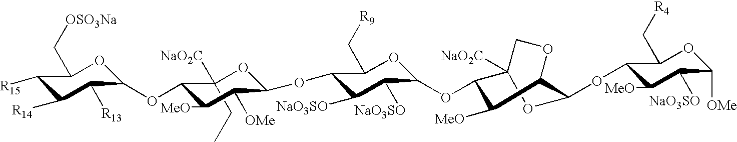

### IC50 Determination of Compounds by Anti-Factor Xa Assay

| | | | | Anti-fXa activity |
|---|---|---|---|---|
| Example | R₁₃/R₁₄/R₁₅ | R₉ | R₄ | IC50 (nM) |
| 9 | OMe | OSO₃Na | N₃ | 40.60 |

Compounds Derived from 5S Templates

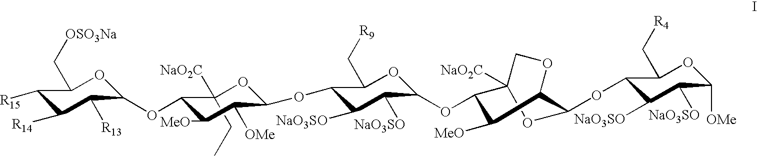

### IC50 Determination of Compounds by Anti-Factor Xa Assay

| | | | | Anti-fXa activity |
|---|---|---|---|---|
| Example | R₁₃/R₁₄/R₁₅ | R₉ | R₄ | IC50 (nM) |
| 25 | OBn | Ohexyl | Ohexyl | 474.00 |
| 51 | OMe | N₃ | OH | 160.10 |
| 53 | OMe | NHDOCA | OSO₃Na | 157.00 |

### O-Alkyl/NHR Family: R₁₄,R₁₅═O-Alkyl/O-Arylalkyl, R₁₃: NHR″

Compounds Derived from 4S Templates

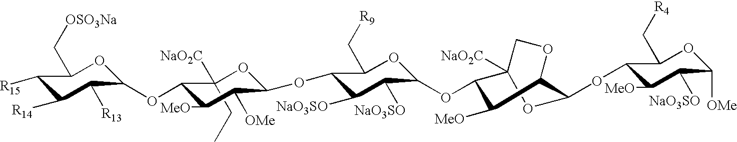

### IC50 Determination of Compounds by Anti-Factor Xa Assay

| | | | | | Anti-fXa |
|---|---|---|---|---|---|
| | | | | | activity |
| Example | R₁₄/R₁₅ | R₁₃ | R₉ | R₄ | IC50 (nM) |
| 61 | OBn | N₃ | OH | OH | 76.80 |
| 67 | OBu | NH(Z-amino) | OH | OH | 181.60 |
| | | hexanoyl | | | |
| 70 | OBu | NH(3- | OH | OH | 219.20 |
| | | cyclopentyl | | | |
| | | propanoyl) | | | |
| 78 | OBu | N₃ | N₃ | OSO₃Na | 136.20 |
| 87 | OHex | NH₂ | OSO₃Na | OSO₃Na | 74.80 |

Compounds Derived from 5S Templates

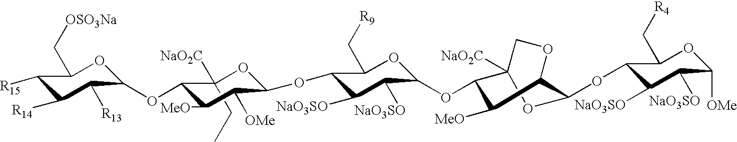

### IC50 Determination of Compounds by Anti-Factor Xa Assay

| | | | | | Anti-fXa |
|---|---|---|---|---|---|
| | | | | | activity |
| Example | R₁₄/R₁₅ | R₁₃ | R₉ | R₄ | IC50 (nM) |
| 89 | OBn | N₃ | OSO₃Na | OSO₃Na | 28.00 |

1. A compound comprising an oligosaccharide of Formula (I): 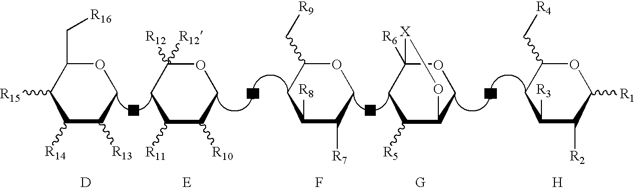
wherein:
R₂, R₇, R₈ and R₁₆ are independently selected from the group consisting of: OSO₃H and NHSO₃H;
R₆ and R₁₂ are each COOH;
R₁, R₃, R₄, R₅, R₉, R₁₀, R₁₁, R₁₃, R₁₄ and R₁₅ are independently selected from the group consisting of: OH, OSO₃H, NH₂, NR′R″, N₃, O-alkyl, O-acyl, O-alkenyl, O-alkynyl, O-aryl, O-heteroaryl, O-heterocyclyl, O-aminoalkyl, O-alkylaryl, O-alkylheteroaryl, O-alkylheterocyclyl;
provided at least one of R₃, R₄, R₉, R₁₀, R₁₃, R₁₄ and R₁₅ is independently selected from the group consisting of: NH₂, NR′R″, N₃, O—(C₄₋₃₀-alkyl), O—(C₄₋₃₀-acyl), O-alkenyl, O-alkynyl, O-aryl, O-heteroaryl, O-heterocyclyl, O-aminoalkyl, O-alkylaryl, O-alkylheteroaryl, O-alkylheterocyclyl;
R_{12′} is selected from the group consisting of: H and alkyl;
X is selected from the group consisting of: CH₂ and CH₂CH₂; and
wherein R′ is independently selected from the group consisting of: H and alkyl;
wherein R″ is independently selected from the group consisting of: H, alkyl, alkenyl, alkoxy, C(O)alkyl, C(O)alkoxy, C(O)aryl, C(O)alkylaryl, C(O)arylalkyl, and a lipophilic delivery moiety; and
wherein any of R′, R″, R₃, R₄, R₉, R₁₀, R₁₃, R₁₄ and R₁₅ are independently optionally substituted with one or more groups independently selected from alkyl, alkoxyalkyl, alkoxyaryl, alkynyl, heteroaryl, aryl, arylalkyl, alkaryl, COOH, COOalkyl, SH, S-alkyl, SO₂H, SO₂alkyl, SO₂aryl, SO₂alkaryl, P(OH)(O)₂, halo, haloalkyl, perhaloalkyl, OH, O-alkyl, ═O, NH₂, ═NH, NHalkyl, N(alkyl)₂, ═Nalkyl, NHC(O)alkyl, C(O)NH₂, C(O)NHalkyl, C(O)N(alkyl)₂, C(O)NHaryl, NO₂, ONO₂, CN, SO₂, SO₂NH₂, C(O)H, C(O)alkyl and wherein any of the aforementioned groups is optionally protected by a suitable protecting group;
or a salt, solvate or prodrug thereof.

2. The compound, salt, solvate or prodrug of claim 1 wherein:
R₃, R₄, R₉, R₁₀, R₁₃, R₁₄ and R₁₅ are independently selected from the group consisting of: OH, OSO₃H, NH₂, NR′R″, N₃, O—(C₄₋₃₀-alkyl), O—(C₄₋₃₀-acyl), O-alkenyl, O-alkynyl, O-aryl, O-heteroaryl, O-heterocyclyl, O-aminoalkyl, O-alkylaryl, O-alkylheteroaryl, O-alkylheterocyclyl;
wherein any of R₃, R₄, R₉, R₁₀, R₁₃, R₁₄ and R₁₅ are independently optionally substituted with one or more groups independently selected from alkyl, alkoxyalkyl, alkoxyaryl, alkynyl, heteroaryl, aryl, arylalkyl, alkaryl, COOH, COOalkyl, SH, S-alkyl, SO₂H, SO₂alkyl, SO₂aryl, SO₂alkaryl, P(OH)(O)₂, halo, haloalkyl, perhaloalkyl, OH, O-alkyl, ═O, NH₂, ═NH, NHalkyl, N(alkyl)₂, ═Nalkyl, NHC(O)alkyl, C(O)NH₂, C(O)NHalkyl, C(O)N(alkyl)₂, NO₂, ONO₂, CN, SO₂, SO₂NH₂, C(O)H, C(O)alkyl and C(O)NHaryl and any of the aforementioned amine containing groups is optionally protected by a benzyloxycarbonyl group.

3. The compound, salt, solvate or prodrug of claim 1 wherein, R₃, R₄, R₉, R₁₀, R₁₃, R₁₄ and R₁₅ are selected from the group consisting of: OH, N₃, NH₂, NR′R″, OSO₃H, O-alkyl, O-alkylaryl, O-arylalkyl and O-acyl;
wherein any of R₃, R₄, R₉, R₁₀, R₁₃, R₁₄ and R₁₅ are independently optionally substituted with one or more groups independently selected from: OH, alkyl, halo, haloalkyl, perhaloalkyl, NH₂, NO₂, ONO₂ and any of the aforementioned amine containing groups is optionally protected by a benzyloxycarbonyl group.

4. The compound, salt, solvate or prodrug of claim 1 wherein R′ is H and R″ is selected from the group consisting of: H, alkyl, alkenyl, alkoxy, C(O)alkyl, C(O)alkoxy, C(O)alkylaryl, C(O)arylalkyl, niflumic acid, mineral corticoids, cholesterol, sodium N-[10-(2-hydroxybenzoyl)amino] decanoate (SNAD) and sodium N-[8-(2-hydroxybenzoyl)amino] caprylate (SNAC);
wherein the R″ group is optionally substituted with one or more groups independently selected from: alkyl, halo, haloalkyl, perhaloalkyl, NH₂, NO₂, ONO₂ and any of the aforementioned amine containing groups is optionally protected by a benzyloxycarbonyl group.

5. The compound, salt, solvate or prodrug of claim 1 wherein R′ is H and R″ is selected from the group consisting of H, (benzyloxycarbonyl)aminohexanoyl, cyclopentylpropanoyl, deoxycholoyl (DOCA), SNAD, SNAC, cholesterol, hexanoyl, hydrocinnamoyl, 3-cyclopentylpropanoyl, 3,5-bis(trifluoromethyl)benzoyl, (4-nitrooxy)butanoyl, dodecanoyl, arachidoyl, aminohexanoyl, niflumic acid.

6. The compound, salt, solvate or prodrug of claim 1 wherein R′ and R″ are both alkyl.

7. The compound, salt, solvate or prodrug of claim 1 wherein R₁, R₅ and R₁₁ are each O-alkyl.

8. The compound, salt, solvate or prodrug of claim 1 wherein:
R₁ and R₁₁ are O-alkyl;
R₂, R₇, R₈ and R₁₆ are OSO₃H;
R₃ is selected from a group consisting of the following: OH, OSO₃H, O-alkyl, O-arylalkyl, and O-acyl wherein any one of the preceding groups is optionally substituted with one or more groups independently selected from: OH, alkyl, halo and perhaloalkyl;
R₆ and R₁₂ are each COOH;
R_{12′} is CH₂CH₃; and
X is CH₂.

9. The compound, salt, solvate or prodrug of claim 1 wherein:
R₁, R₅, R₁₀ and R₁₁ are O-alkyl;
R₂, R₇, R₈ and R₁₆ are OSO₃H;
R₃ is selected from OSO₃H or O-alkyl;
R₆ and R₁₂ are each COOH;
R_{12′} is CH₂CH₃; and
X is CH₂.

10. The compound, salt, solvate or prodrug of claim 1 wherein R₁₄ and R₁₅ are selected from any one of the following groups: OH, O-arylalkyl and O-alkylaryl;
wherein any of R₁₄ and R₁₅ are optionally substituted with one or more groups independently selected from: alkyl, halo, haloalkyl, perhaloalkyl, NO₂, ONO₂ and any of the aforementioned amine containing groups is optionally protected by a benzyloxycarbonyl group.

11. The compound, salt, solvate or prodrug of claim 1 wherein R₁₃ is selected from any one of the following groups: OH, O-arylalkyl, O-alkyl, N₃, NH₂, NR′R″;
wherein any of R″, R′ and R₁₃ are optionally substituted with one or more groups independently selected from: alkyl, halo, haloalkyl, perhaloalkyl, NO₂, ONO₂ and any of the aforementioned amine containing groups is optionally protected by a benzyloxycarbonyl group.

12. The compound, salt, solvate or prodrug of claim 1 wherein R₉ is selected from any one of the following groups: OH, O-alkyl, O-acyl, NH₂, N₃, NR′R″, OSO₃H, O-arylalkyl and O-alkylaryl;
wherein any of R″, R′ and R₉ are optionally substituted with one or more groups independently selected from: alkyl, halo, haloalkyl, perhaloalkyl, NO₂, ONO₂ and any of the aforementioned amine containing groups is optionally protected by a benzyloxycarbonyl group.

13. The compound, salt, solvate or prodrug of claim 1 wherein R₄ is selected from any one of the following groups: OH, O-alkyl, O-acyl, NH₂, N₃, NR′R″, OSO₃H, O-arylalkyl and O-alkylaryl;
wherein any of R″, R′ and R₄ are optionally substituted with one or more groups independently selected from: alkyl, halo, haloalkyl, perhaloalkyl, NO₂, ONO₂ and any of the aforementioned amine containing groups is optionally protected by a benzyloxycarbonyl group.

14. The compound, salt, solvate or prodrug of claim 1 wherein:
R₃ is OSO₃H;
R₁₀ is OCH₃;
R₁₃ is NH₂; and
R₄, R₉, R₁₄ and R₁₅ are each OH.

15. The compound, salt, solvate or prodrug of claim 1 wherein monosaccharide unit G of the oligosaccharide has the following conformation:

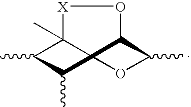

16. The compound, salt, solvate or prodrug of claim 1 wherein monosaccharide units D, E, F and H of the oligosaccharide have the D-gluco stereochemistry as follows:

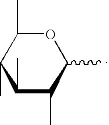

17. The compound, salt, solvate or prodrug of claim 1 wherein monosaccharide unit G of the oligosaccharide has the following stereochemistry:

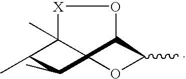

18. The compound, salt, solvate or prodrug of claim 1 wherein R₁, R₅ and R₁₁ are each OMe.

19. The compound, salt, solvate or prodrug of claim 1 wherein R₂, R₇, R₈ and R₁₆ are each OSO₃H.

20. The compound, salt, solvate or prodrug of claim 1 wherein X is CH₂.

21. The compound, salt, solvate or prodrug of claim 1 wherein R_{12′} is CH₂CH₃.

22. The compound, salt, solvate or prodrug of claim 1 wherein any of R₃, R₄, R₉, R₁₀, R₁₃, R₁₄ and R₁₅ are independently selected from: O-butyl, nonanoyl, (4-tert-butyl)benzyloxy, 3-cyclopentylpropanoyl, hexanoyl, 2,2-dimethylpropyloxy, 4-chlorobenzyloxy, OH and deoxycholoyl.

23. The compound, salt, solvate or prodrug of claim 1 wherein the oligosaccharide is of Formula (II):

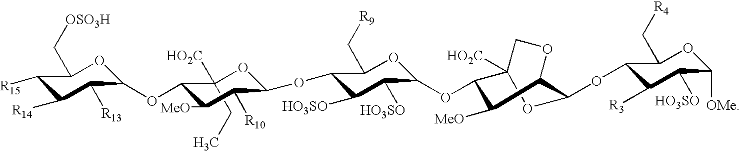

24. The compound, salt, solvate or prodrug of claim 1 wherein R₁₀ is OCH₃.

25. The compound, salt, solvate or prodrug of claim 1 wherein R₃ is selected from OSO₃H or OMe.

26. The compound, salt, solvate or prodrug of claim 23 wherein R₁₄ and R₁₅ are selected from any one of the following groups: OH, O-alkyl and O-arylalkyl.

27. The compound, salt, solvate or prodrug of claim 23 wherein R₁₃ is selected from any one of the following groups: O-arylalkyl, O-alkyl, N₃, and NR′R″;
wherein R′ is selected from H; and
R″ is selected from any one of the following: C(O)alkyl, C(O)arylalkyl and H, wherein any of the aforementioned groups is optionally substituted with one or more NH₂ groups optionally protected by a benzyloxycarbonyl group.

28. The compound, salt, solvate or prodrug of claim 23 wherein R₉ is selected from any one of the following groups: OH, O-alkyl, N₃, NR′R″, OSO₃H and O-arylalkyl; and
wherein R′ is H and R″ is selected from DOCA.

29. The compound, salt, solvate or prodrug of claim 23 wherein R₄ is selected from any one of the following groups: OH, O-alkyl, N₃, NR′R″ and OSO₃H;
wherein R′ is selected from H; and R″ is selected from C(O)arylalkyl.

30. The salt of claim 1 or 23 wherein the counter-ion is selected from the group consisting of: sodium and potassium.

31. A pharmaceutical composition comprising a compound, salt, solvate or prodrug according to claim 1 or 23 and a pharmaceutically acceptable diluent or carrier.

32. A method of making a pharmaceutical composition according to claim 31, comprising mixing said compound, salt, solvate or pro-drug with a pharmaceutically acceptable diluent or carrier.

33-34. (canceled)

35. A method of treating a blood clotting disorder in a human or animal subject comprising administering to the human or animal subject a therapeutically effective amount of a compound, salt, solvate or prodrug as defined in claim 1 or 23.

36. The method of claim 35, wherein the compound, salt, solvate or prodrug is orally administered.

37. The method of claim 35, wherein the blood clotting disorder is selected from: deep vein thromboembolism including deep vein thrombosis and pulmonary embolism, post surgical prophylaxis of deep venous thrombosis, coronary syndromes, myocardial infarction and stroke.
